SEQUENCE LISTING (48 sequences)

SEQ ID NO: 1 - protein, 5 aa, Artificial Sequence
  Synthetic Construct
SFAMS

SEQ ID NO: 2 - protein, 17 aa, Artificial Sequence
  Synthetic Construct
AISGSGGGTYYADSVKG

SEQ ID NO: 3 - protein, 13 aa, Artificial Sequence
  Synthetic Construct
DKILWFGEPVFDY

SEQ ID NO: 4 - protein, 11 aa, Artificial Sequence
  Synthetic Construct
RASQSVSSYLA

SEQ ID NO: 5 - protein, 7 aa, Artificial Sequence
  Synthetic Construct
DASNRAT

SEQ ID NO: 6 - protein, 10 aa, Artificial Sequence
  Synthetic Construct
QQRSNWPPTF

SEQ ID NO: 7 - protein, 23 aa, Artificial Sequence
  Synthetic Construct
EIVLTQSPATLSLSPGERATLSC

SEQ ID NO: 8 - protein, 15 aa, Artificial Sequence
  Synthetic Construct
WYQQKPGQAPRLLIY

SEQ ID NO: 9 - protein, 32 aa, Artificial Sequence
  Synthetic Construct
GIPARFSGSGSGTDFTLTISSLEPEDFAVYYC

SEQ ID NO: 10 - protein, 9 aa, Artificial Sequence
  Synthetic Construct
GQGTKVEIK

SEQ ID NO: 11 - protein, 30 aa, Artificial Sequence
  Synthetic Construct
EVQLLESGGGLVQPGGSLRLSCAVSGFTFN

SEQ ID NO: 12 - protein, 14 aa, Artificial Sequence
  Synthetic Construct
WVRQAPGKGLEWVS

SEQ ID NO: 13 - protein, 32 aa, Artificial Sequence
  Synthetic Construct
RFTISRDNSKNTLYLQMNSLRAEDTAVYFCAK

SEQ ID NO: 14 - protein, 11 aa, Artificial Sequence
  Synthetic Construct
WGQGTLVTVSS

SEQ ID NO: 15 - protein, 122 aa, Artificial Sequence [sequence omitted]

SEQ ID NO: 16 - protein, 107 aa, Artificial Sequence [sequence omitted]

SEQ ID NO: 17 - protein, 10 aa, Artificial Sequence
  Synthetic Construct
GFTFSDSWIH

SEQ ID NO: 18 - protein, 18 aa, Artificial Sequence
  Synthetic Construct
AWISPYGGSTYYADSVKG

SEQ ID NO: 19 - protein, 9 aa, Artificial Sequence
  Synthetic Construct
RHWPGGFDY

SEQ ID NO: 20 - protein, 11 aa, Artificial Sequence
  Synthetic Construct
RASQDVSTAVA

SEQ ID NO: 21 - protein, 7 aa, Artificial Sequence
  Synthetic Construct
SASFLYS

SEQ ID NO: 22 - protein, 9 aa, Artificial Sequence
  Synthetic Construct
QQYLYHPAT

SEQ ID NO: 23 - protein, 118 aa, Artificial Sequence [sequence omitted]

SEQ ID NO: 24 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 25 - DNA, 5620 bp, Homo sapiens
gcagtttcagaacccagccagcctctctcttgctgcctagcctcctgccggcctcatcttcgcccagccaaccccgcctggagccctatggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctgagccagtcgctgtggttgttttagctccttgactccttgtggtttatgtcatcatacatgactcagcatacctgctggtgcagagctgaagattttggagggtcctccacaataaggtcaatgccagagacggaagcctttttccccaaagtcttaaaataacttatatcatcagcatacctttattgtgatctatcaatagtcaagaaaaattattgtataagattagaatgaaaattgtatgttaagttacttcactttaattctcatgtgatccttttatgttatttatatattggtaacatcctttctattgaaaaatcaccacaccaaacctctcttattagaacaggcaagtgaagaaaagtgaatgctcaagtttttcagaaagcattacatttccaaatgaatgaccttgttgcatgatgtatttttgtacccttcctacagatagtcaaaccataaacttcatggtcatgggtcatgttggtgaaaattattctgtaggatataagctacccacgtacttggtgctttaccccaacccttccaacagtgctgtgaggttggtattatttcattttttagatgagaaaatgggagctcagagaggttatatatttaagttggtgcaaaagtaattgcaagttttgccaccgaaaggaatggcaaaaccacaattatttttgaaccaacctaataatttaccgtaagtcctacatttagtatcaagctagagactgaatttgaactcaactctgtccaactccaaaattcatgtgctttttccttctaggcctttcataccaaactaatagtagtttatattctcttccaacaaatgcatattggattaaattgactagaatggaatctggaatatagttcttctggatggctccaaaacacatgtttttcttcccccgtcttcctcctcctcttcatgctcagtgttttatatatgtagtatacagttaaaatatacttgttgctggtactggcagcttatattttctctcttttttcatggattaaccttgcttgagggctttaacaattgtattactttttcaaagaactaagctttagcttcattgatttttttctatttaattgggttttgctcttctctttagcattggaaacatagaaatgctttctgatttctttgggtagatttacgtattcagcttcttgagatggaagtttagatcactgatccttcagcttgttttcttttttgtatacatagattttaggacgatatattttcccttgagttctgctttagctgcagctcttatgttttgatatgcctctctttattatccttcagttaaaaatatctttcaattcattgttatataaaaatatgtgcctagtttttaacatctggagattttctagttttgaaaaaaacataagccaggcatggtggctcacacctgtatccccagcactttgggaggccgagacgggaggatcgcctgagctcaggagtttttacaccagcctgggaataacagtgagacattatctccaaaaaaattacctgggtatggtgttgtgcacctgtagtcccagctactctggagactgaggtgggaggattgtttgagcttgggaggttgaggctgcagggagctgtgatcacaccactgcactctggcctgagtgacagattgagaccctgtctcaataaaagcaaaaataaagaaaataaaccatatgtgttgaacaaaggattaataaattaatttgagactccttcagggaatgaccacaatttattgaaaatagcctaaatgttggagtcaggcatttctggattcatattttgacatcatgctgtcatcttgaacaaaatgcctaacctttctgaacttcaacttccttgccactcaaataaggattacaaaacttaaaatgtggtaagtactaaagacgacagcaaaaattgagtccagcacagagcttcctaaataagcaagcactcaacagagttggttcctttcttcctcccctgcttgacaatccagtttcccacaggagcctttgtagctgtagccaccatggtcagtccagggattcttcactagccccttctcccctggcagacatccttgtgggagtttagtcttggctcgacatgaggatgggggtttgggaccagttctgagtgagaatcagacttgccccaagttgccattagctccccctgcagaatgtcttcagaatcggggcccggtcagtctcctgggtgacctgctgttttcctcttaagatcctttccactttggttgctgctttcgggactcatcgagtccttgctcaacaggataccccttgaagtggctgcctgggccacatccccttccaaacaagaaatcaaaatattagaaatcaatttttgaaatttcccctaggaagactcatttgagtgttcaagttcagagccagtggagaccttaggggagggtggtcacaaggattttgcacagtgctttagagggtcccagggagccacagaggtggtgaggggctgggtgctcttttctccgtgcatgaccttgtgtgtctatcttcattaccacaatgcctcatctctacctcctttccccctgtagttccaacgtgggtatctttgccatctctggcccgaaggactttctgacctacatgtataaataccccctcacaatatatattacttttcctataagtgacttctctactggattactggttgctcatacacctcatattttactcgtaaatctactactccctgtctgcctactccattctcatttgctgtagaaaattctcttaccatcccaactttcacccaccatcatgcttacccaaaggctgtgggaatgacctgggccctaatgccccttttctaaattcctaaggctcaccattttcctattgtaatggttcttgaccttataatgtttgaggcaccttttcaaatatagtcctttgatttcagactgaatacttgaaaggacacacacacacatacgtaagtgcatatgactgcatacacccacacacacacacgtgcctgtatacagtcatatgatacatacacaaacacacgcacacaagcctgcatacatcatatgccaacagtggggatatgttctgagaaatgcatcattagatgattttgtcattgtgtgaacatcatagagtgtacttacactaacctagatggtctaacctactacacacccaggctacatggtatcacctattcctcctaggctacaagcctgtacagcgtgtgtctgtactaaatgctgtgggcaattttaacctgatggtaaatgtttgtgtatctaaacatatctaaacatagaaaaggtacagtaaacatgcagtattataatcttatgagaccgtcatcatatatgtggtccactgtttgggccatcattggctgaaaagtggttatgcgacacatgactgtatatatactttcctgttacaacaacagtgtctctcaatccacagtaattgcagcatccagtaggtcttactttagccctgagtcaccatttgtgtcaacgtgtttagtgccatgtccacgtctctcatgtaactggcagagctatcaaatattttggcaaaacacattgtttctttggctttgccttggtaactttctgtgccttttgtagctcttgtttggaagaagctcaacccatgtctgcacactgtgatacaagggggacagcatcgacatcgacttacttcttggtgccttattcctccttagaacaattcctaaatctgtaacttaagtttctcaggaagattccatactgcacagaaaactgcttttgtgggtttttaaaaggcaagttgttatatgtgctggatagtttttaagtatgacataaaaattgtataaagtaaaatattaaaatacacctagaatactgtataactttaagtcattttatcaacacattgctaatccagatattttcccgcagtttttctttgaataacagagcaattaatttacttttactatgaagagtcatcattttagtatgtattttaagcaatccaccaagaactcagtaggcagctgagaggtgctgcccagagaagtggtgattagcttggccttagctcacccacacaaagcacaacaggctttgaactattccctaacggggcatttattcttttttttttttttttttgggagacggagtctcgctgtcgcccaggctagagtgcagtggcgcgatctcggctcactgcaggctccaccccctggggttcacgccattctcctgcctcagcctcccaagtagctgggactgcaggcgcccgccatctcgcccggctaattttttgtatttttagtagagacggggtttcaccgtgttagccaggatagggcatttattcttgaacttgattcagagaggcacacattaccattctctaatcagaatgcaagtagcgcaaggcggtggaaactatggaattcggaggcaggtgatgcattgggcgagtttattaacatctgtgactctctagtttgaaatttatttgtaacagacaaaaatgaattaaacaaacaataaaagtataataaagaa

SEQ ID NO: 26 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 27 - protein, 10 aa, Artificial Sequence
  Synthetic Construct
  MISC_FEATURE (6)..(6): Xaa is Asp or Gly
GFTFSXSWIH

SEQ ID NO: 28 - protein, 18 aa, Artificial Sequence
  Synthetic Construct
  MISC_FEATURE (4)..(4): Xaa is Ser or Leu
  MISC_FEATURE (10)..(10): Xaa is Thr or Ser
AWIXPYGGSXYYADSVKG

SEQ ID NO: 29 - protein, 25 aa, Artificial Sequence [sequence omitted]

SEQ ID NO: 30 - protein, 13 aa, Artificial Sequence
  Synthetic Construct
WVRQAPGKGLEWV

SEQ ID NO: 31 - protein, 32 aa, Artificial Sequence
  Synthetic Construct
RFTISADTSKNTAYLQMNSLRAEDTAVYYCAR

SEQ ID NO: 32 - protein, 11 aa, Artificial Sequence
  Synthetic Construct
  MISC_FEATURE (5)..(5): Xaa is Asp or Val
  MISC_FEATURE (6)..(6): Xaa is Val or Ile
  MISC_FEATURE (7)..(7): Xaa is Ser or Asn
  MISC_FEATURE (9)..(9): Xaa is Ala or Phe
  MISC_FEATURE (10)..(10): Xaa is Val or Leu
RASQXXXTXXA

SEQ ID NO: 33 - protein, 7 aa, Artificial Sequence
  Synthetic Construct
  MISC_FEATURE (4)..(4): Xaa is Phe or Thr
  MISC_FEATURE (6)..(6): Xaa is Tyr or Ala
SASXLXS

SEQ ID NO: 34 - protein, 9 aa, Artificial Sequence
  Synthetic Construct
  MISC_FEATURE (3)..(3): Xaa is Tyr, Gly, Phe, or Ser
  MISC_FEATURE (4)..(4): Xaa is Leu, Tyr, Phe, or Trp
  MISC_FEATURE (5)..(5): Xaa is Tyr, Asn, Ala, Thr, Gly, Phe, or Ile
  MISC_FEATURE (6)..(6): Xaa is His, Val, Pro, Thr, or Ile
  MISC_FEATURE (8)..(8): Xaa is Ala, Trp, Arg, Pro, or Thr
QQXXXXPXT

SEQ ID NO: 35 - protein, 23 aa, Artificial Sequence
  Synthetic Construct
DIQMTQSPSSLSASVGDRVTITC

SEQ ID NO: 36 - protein, 15 aa, Artificial Sequence
  Synthetic Construct
WYQQKPGKAPKLLIY

SEQ ID NO: 37 - protein, 32 aa, Artificial Sequence
  Synthetic Construct
GVPSRFSGSGSGTDFTLTISSLQPEDFATYYC

SEQ ID NO: 38 - protein, 11 aa, Artificial Sequence
  Synthetic Construct
FGQGTKVEIKR

SEQ ID NO: 39 - protein, 30 aa, Artificial Sequence
  Synthetic Construct
EVQLVESGGGLVQPGGSLRLSCAASGFTFS

SEQ ID NO: 40 - protein, 14 aa, Artificial Sequence
  Synthetic Construct
WVRQAPGKGLEWVA

SEQ ID NO: 41 - protein, 10 aa, Artificial Sequence
  Synthetic Construct
FGQGTKVEIK

SEQ ID NO: 42 - protein, 15 aa, Artificial Sequence
  Synthetic Construct
WGQGTLVTVSSASTK

SEQ ID NO: 43 - protein, 122 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTK

SEQ ID NO: 44 - protein, 108 aa, Artificial Sequence; identical to SEQ ID NO: 24
  Synthetic Construct

SEQ ID NO: 45 - protein, 447 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 440 aa, Artificial Sequence
  Synthetic Construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 214 aa, Artificial Sequence
  Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0389103 A1
(43) Pub. Date: Dec. 8, 2022

(54) DIAGNOSTIC AND THERAPEUTIC METHODS FOR TREATMENT OF HEMATOLOGIC CANCERS

(71) Applicants: Genentech, Inc., South San Francisco, CA (US); F. Hoffmann-La Roche AG, Basel (CH)

(72) Inventors: Huang HUANG, Mississauga (CA); Aparna RAVAL, South San Francisco, CA (US)

(21) Appl. No.: 17/774,952

(22) PCT Filed: Nov. 5, 2020
(86) PCT No.: PCT/US2020/059107

§ 371 Date: May 6, 2022

Related U.S. Application Data
(60) Provisional application No. 62/960,521, filed on Jan. 13, 2020.
(60) Provisional application No. 62/931,574, filed on Nov. 6, 2019.

(51) Int. Cl. C07K 16/28; G01N 33/574; A61P 35/00

(52) U.S. Cl. CPC C07K 16/2827; G01N 33/57407; C07K 16/2896; A61P 35/00; C07K 2317/24; A61K 2039/507

(57) ABSTRACT

Disclosed herein are diagnostic and therapeutic methods for the treatment of hematologic cancers, including multiple myeloma (MM), as well as related compositions. In particular, the invention relates to diagnostic and therapeutic methods for treatments involving a PD-L1 axis binding antagonist (e.g., an anti-PD-L1 antibody, e.g., atezolizumab) and an anti-CD38 antibody (e.g., an anti-CD38 antagonist antibody, e.g., daratumumab) for use in treating hematologic cancer (e.g., a multiple myeloma (MM), e.g., a relapsed or refractory MM).

### CROSS-REFERENCE TO RELATED APPLICATIONS

This application claims benefit to U.S. Provisional Application No. 62/931,574, filed on Nov. 6, 2019, and U.S. Provisional Application No. 62/960,521, filed on Jan. 13, 2020, which are incorporated by reference herein in their entirety.

### SEQUENCE LISTING

The instant application contains a Sequence Listing which has been submitted electronically in ASCII format and is hereby incorporated by reference in its entirety. Said ASCII copy, created on Nov. 3, 2020, is named 51177-028WO3_Sequence_Listing_11.3.20_ST25 and is 38,756 bytes in size.

### FIELD OF THE INVENTION

Provided herein are methods and compositions for use in treating a hematologic cancer (e.g., a myeloma (e.g., a multiple myeloma (MM), e.g., a relapsed or refractory MM). In particular, the invention provides biomarkers for patient identification, selection, and treatment.

### BACKGROUND OF THE INVENTION

Cancer remains one of the deadliest threats to human health. In the U.S., cancer affects nearly 1.3 million new patients each year and is the second leading cause of death after heart disease, accounting for approximately 1 in 4 deaths. It is also predicted that cancer may surpass cardiovascular diseases as the number one cause of death within 5 years. A hematologic cancer, multiple myeloma (MM), affects almost 20,000 people every year in the United States, and worldwide, approximately 160,000 people are diagnosed with MM annually. MM remains incurable despite advances in treatment, with an estimated median survival of 8-10 years for standard-risk myeloma and 2-3 years for high-risk disease.

Studies in humans with immune checkpoint inhibitors have demonstrated the promise of harnessing the immune system to control and eradicate tumor growth. The programmed death 1 (PD-1) receptor and its ligand programmed death-ligand 1 (PD-L1) are immune checkpoint proteins that have been implicated in the suppression of immune system responses during chronic infections, pregnancy, tissue allografts, autoimmune diseases, and cancer. PD-L1 regulates the immune response by binding to the inhibitory receptor PD-1, which is expressed on the surface of T-cells, B-cells, and monocytes. PD-L1 negatively regulates T-cell function also through interaction with another receptor, B7-1. Formation of the PD-L1/PD-1 and PD-L1/B7-1 complexes negatively regulates T-cell receptor signaling, resulting in the subsequent downregulation of T-cell activation and suppression of anti-tumor immune activity.

Despite significant advancement in the treatment of cancer (e.g., myeloma, e.g., multiple myeloma (MM), e.g., a relapsed or refractory MM), improved therapies and diagnostic methods are still being sought.

### SUMMARY OF THE INVENTION

The present invention relates to diagnostic and therapeutic methods for the treatment of hematologic cancers (e.g., a myeloma (e.g., a multiple myeloma (MM), e.g., a relapsed or refractory MM).

In one aspect, the disclosure features a method of identifying an individual having a hematologic cancer who may benefit from a treatment including a PD-L1 axis binding antagonist and an anti-CD38 antibody, the method including determining an osteoclast number in a tumor sample obtained from the individual, wherein an osteoclast number that is lower than a reference osteoclast number identifies the individual as one who may benefit from the treatment.

In some aspects, the osteoclast number in the tumor sample is the number of osteoclasts within a tumor region. In some aspects, the tumor region includes an area including tumor cells and adjacent myeloid cells. In some aspects, the tumor region does not comprise fat bodies and bone trabeculae. In some aspects, the tumor region includes an area within about 40 μm to about 1 mm of a tumor cell or a myeloid cell adjacent to a tumor cell.

In some aspects, the osteoclast number in the tumor sample is lower than the reference osteoclast number and the method further includes administering to the individual a treatment including a PD-L1 axis binding antagonist and an anti-CD38 antibody.

In another aspect, the disclosure features a method of treating an individual having a hematologic cancer, the method including: (a) determining an osteoclast number in a tumor sample obtained from the individual, wherein the osteoclast number in the tumor sample has been determined to be lower than a reference osteoclast number; and (b) administering an effective amount of a PD-L1 axis binding antagonist and an anti-CD38 antibody to the individual based on the osteoclast number in the tumor sample determined in step (a).

In another aspect, the disclosure features a method of treating an individual having a hematologic cancer, the method including administering to the individual an effective amount of a PD-L1 axis binding antagonist and an anti-CD38 antibody, wherein prior to treatment an osteoclast number in a tumor sample obtained from the individual has been determined to be lower than a reference osteoclast number.

In some aspects, the reference osteoclast number is a baseline osteoclast number in a reference population of individuals having the hematologic cancer, the reference population consisting of individuals who have been treated with a PD-L1 axis binding antagonist and an anti-CD38 antibody. In some aspects, the reference osteoclast number significantly separates a first subset of individuals from a second subset of individuals in the reference population based on a significant difference in responsiveness to treatment with the PD-L1 axis binding antagonist and the anti-CD38 antibody. In some aspects, responsiveness to treatment is in terms of an objective response. In some aspects, the objective response is a stringent complete response (sCR), a complete response (CR), a very good partial response (VGPR), a partial response (PR), or a minimal response (MR).

In some aspects, the reference osteoclast number is a pre-assigned osteoclast number.

In some aspects, the method includes administering to the individual the anti-CD38 antibody intravenously.

In some aspects, the method includes administering to the individual the anti-CD38 antibody at a dose of about 16 mg/kg.

In another aspect, the disclosure features a method of identifying an individual having a hematologic cancer who may benefit from a treatment including a PD-L1 axis binding antagonist and an anti-CD38 antibody, the method including determining a CD8⁺ T cell density in a tumor sample obtained from the individual, wherein a CD8⁺ T cell density that is higher than a reference CD8⁺ T cell density identifies the individual as one who is more likely to benefit from the treatment.

In some aspects, the CD8⁺ T cell density in the tumor sample is the density of CD8⁺ T cells within a tumor cluster. In some aspects, the tumor cluster is an area including adjacent tumor cells. In some aspects, the tumor cluster is at least about 25 μm to about 400 μm in length along its longest axis.

In some aspects, the CD8⁺ T cell density in the tumor sample is higher than the reference CD8⁺ T cell density and the method further includes administering to the individual a treatment including a PD-L1 axis binding antagonist and an anti-CD38 antibody.

In another aspect, the disclosure features a method of treating an individual having a hematologic cancer, the method including: (a) determining a CD8⁺ T cell density in a tumor sample obtained from the individual, wherein the CD8⁺ T cell density in the tumor sample has been determined to be higher than a reference CD8⁺ T cell density; and (b) administering an effective amount of a PD-L1 axis binding antagonist and an anti-CD38 antibody to the individual based on the CD8⁺ T cell density in the tumor sample determined in step (a).

In another aspect, the disclosure features a method of treating an individual having a hematologic cancer, the method including administering to the individual an effective amount of a PD-L1 axis binding antagonist and an anti-CD38 antibody, wherein prior to treatment a CD8⁺ T cell density in a tumor sample obtained from the individual has been determined to be higher than a reference CD8⁺ T cell density.

In some aspects, the reference CD8⁺ T cell density is a baseline density of CD8⁺ T cells within tumor clusters in a reference population of individuals having the hematologic cancer, the reference population consisting of individuals who have been treated with a PD-L1 axis binding antagonist and an anti-CD38 antibody. In some aspects, the reference CD8⁺ T cell density significantly separates a first subset of individuals from a second subset of individuals in the reference population based on a significant difference in responsiveness to treatment with the PD-L1 axis binding antagonist and the anti-CD38 antibody.

In some aspects, the reference CD8⁺ T cell density is a pre-assigned CD8⁺ T cell density.

In some aspects, the individual has not been previously administered a treatment including a PD-L1 axis binding antagonist. In some aspects, the individual has not been previously administered a treatment including a PD-L1 axis binding antagonist and an anti-CD38 antibody.

In some aspects, responsiveness to treatment is in terms of an objective response. In some aspects, the objective response is a stringent complete response (sCR), a complete response (CR), a very good partial response (VGPR), a partial response (PR), or a minimal response (MR).

In another aspect, the disclosure features a method of monitoring responsiveness of an individual having a hematologic cancer to a treatment including a PD-L1 axis binding antagonist and an anti-CD38 antibody, the method including: (a) determining, in a biological sample obtained from the individual at a time point following administration of the PD-L1 axis binding antagonist and the anti-CD38 antibody, the number of activated CD8⁺ T cells in the bone marrow; and (b) comparing the number of activated CD8⁺ T cells in the biological sample to a reference number of activated CD8⁺ T cells, wherein an increase in the number of activated CD8⁺ T cells in the biological sample relative to the reference number of activated CD8⁺ T cells indicates that the individual is responding to the treatment.

In some aspects, the number of activated CD8⁺ T cells in the biological sample is increased relative to the reference number of activated CD8⁺ T cells. In some aspects, the method includes administering a further dose of the PD-L1 axis binding antagonist and the anti-CD38 antibody to the individual based on the increase in the number of activated CD8⁺ T cells in the biological sample determined in step (b).

In some aspects, the reference number of activated CD8⁺ T cells is (i) the number of activated CD8⁺ T cells in a biological sample from the individual obtained prior to administration of the PD-L1 axis binding antagonist and the anti-CD38 antibody, (ii) the number of activated CD8⁺ T cells in a biological sample obtained from the individual at a previous time point, wherein the previous time point is following administration of the PD-L1 axis binding antagonist and the anti-CD38 antibody; or (iii) a pre-assigned number of activated CD8⁺ T cells.

In some aspects, the biological sample is a bone marrow aspirate.

In some aspects, responsiveness to treatment is in terms of an objective response. In some aspects, the objective response is a stringent complete response (sCR), a complete response (CR), a very good partial response (VGPR), a partial response (PR), or a minimal response (MR).

In some aspects, the hematologic cancer is a myeloma. In some aspects, the myeloma is a multiple myeloma (MM). In some aspects, the MM is a relapsed or refractory MM.

In some aspects, the anti-CD38 antibody is an anti-CD38 antagonist antibody.

In some aspects, the anti-CD38 antibody includes the following complementarity determining regions (CDRs): (a) a CDR-H1 including the amino acid sequence of SFAMS (SEQ ID NO: 1); (b) a CDR-H2 including the amino acid sequence of AISGSGGGTYYADSVKG (SEQ ID NO: 2); (c) a CDR-H3 including the amino acid sequence of DKILWFGEPVFDY (SEQ ID NO: 3); (d) a CDR-L1 including the amino acid sequence of RASQSVSSYLA (SEQ ID NO: 4); (e) a CDR-L2 including the amino acid sequence of DASNRAT (SEQ ID NO: 5); and (f) a CDR-L3 including the amino acid sequence of QQRSNWPPTF (SEQ ID NO: 6). In some aspects, the anti-CD38 antibody includes the following light chain variable region framework regions (FRs): (a) an FR-L1 including the amino acid sequence of EIVLTQSPATLSLSPGERATLSC (SEQ ID NO: 7); (b) an FR-L2 including the amino acid sequence of WYQQKPGQAPRLLIY (SEQ ID NO: 8); (c) an FR-L3 including the amino acid sequence of GIPARFSGSGSGTDFTLTISSLEPEDFAVYYC (SEQ ID NO: 9); and (d) an FR-L4 including the amino acid sequence of GQGTKVEIK (SEQ ID NO: 10). In some aspects, the anti-CD38 antibody includes the following heavy chain variable region FRs: (a) an FR-H1 including the amino acid sequence of EVQLLESGGGLVQPGGSLRLSCAVSGFTFN (SEQ ID NO: 11); (b) an FR-H2 including the amino acid sequence of WVRQAPGKGLEWVS (SEQ ID NO: 12); (c) an FR-H3 including the amino acid sequence of RFTISRDNSKNTLYLQMNSLRAEDTAVYFCAK (SEQ ID NO: 13); and (d) an FR-H4 including the amino acid sequence of WGQGTLVTVSS (SEQ ID NO: 14). In some aspects, the anti-CD38 antibody includes: (a) a heavy chain variable (VH) domain including an amino acid sequence having at least 95% sequence identity to the amino acid sequence of EVQLLESGGGLVQPGGSLRLSCAVSGFTFNSFAMSWVRQAPGKGLEWVSAISGSGGGT YYADSVKGRFTISRDNSKNTLYLQMNSLRAEDTAVYFCAKDKILWFGEPVFDYWGQGTLVTVSS (SEQ ID NO: 15); (b) a light chain variable (VL) domain including an amino acid sequence having at least 95% sequence identity to the amino acid sequence of EIVLTQSPATLSLSPGERATLSCRASQSVSSYLAWYQQKPGQAPRLLIYDASNRATGIP ARFSGSGSGTDFTLTISSLEPEDFAVYYCQQRSNWPPTFGQGTKVEIK (SEQ ID NO: 16); or (c) a VH domain as in (a) and a VL domain as in (b). In some aspects, the anti-CD38 antibody includes: (a) a VH domain including the amino acid sequence of SEQ ID NO: 15; and (b) a VL domain including the amino acid sequence of SEQ ID NO: 16.

In some aspects, the anti-CD38 antibody is a monoclonal antibody.

In some aspects, the anti-CD38 antibody is a human antibody.

In some aspects, the anti-CD38 antibody is a full-length antibody.

In some aspects, the anti-CD38 antibody is daratumumab.

In some aspects, the anti-CD38 antibody is an antibody fragment that binds CD38 selected from the group consisting of Fab, Fab′, Fab′-SH, Fv, single chain variable fragment (scFv), and (Fab′)₂ fragments.

In some aspects, the anti-CD38 antibody is an IgG class antibody. In some aspects, the IgG class antibody is an IgG1 subclass antibody.

In some aspects, the method includes administering to the individual the anti-CD38 antibody intravenously.

In some aspects, the method includes administering to the individual the anti-CD38 antibody at a dose of about 16 mg/kg.

In some aspects, the PD-L1 axis binding antagonist is selected from the group consisting of a PD-L1 binding antagonist, a PD-1 binding antagonist, and a PD-L2 binding antagonist. In some aspects, the PD-L1 axis binding antagonist is a PD-L1 binding antagonist. In some aspects, the PD-L1 binding antagonist inhibits the binding of PD-L1 to one or more of its ligand binding partners. In some aspects, the PD-L1 binding antagonist inhibits the binding of PD-L1 to PD-1, B7-1, or both PD-1 and B7-1.

In some aspects, the PD-1 binding antagonist is an anti-PD-1 antibody. In some aspects, the anti-PD-1 antibody is MDX-1106 (nivolumab), MK-3475 (pembrolizumab), MEDI-0680 (AMP-514), PDR001, REGN2810, or BGB-108.

In some aspects, the PD-1 binding antagonist is an Fc fusion protein. In some aspects, the Fc fusion protein is AMP-224.

In some aspects, the PD-L1 binding antagonist is an anti-PD-L1 antibody. In some aspects, the anti-PD-L1 antibody is atezolizumab (TECENTRIQ®), MDX-1105, MEDI4736 (durvalumab), or MSB0010718C (avelumab). In some aspects, the anti-PD-L1 antibody is atezolizumab. In some aspects, the anti-PD-L1 antibody includes the following hypervariable regions (HVRs): (a) an HVR-H1 sequence of GFTFSDSWIH (SEQ ID NO: 17); (b) an HVR-H2 sequence of AWISPYGGSTYYADSVKG (SEQ ID NO: 18); (c) an HVR-H3 sequence of RHWPGGFDY (SEQ ID NO: 19); (d) an HVR-L1 sequence of RASQDVSTAVA (SEQ ID NO: 20); (e) an HVR-L2 sequence of SASFLYS (SEQ ID NO: 21); and (f) an HVR-L3 sequence of QQYLYHPAT (SEQ ID NO: 22). In some aspects, the anti-PD-L1 antibody includes: (a) a heavy chain variable (VH) domain including an amino acid sequence having at least 90% sequence identity to the amino acid sequence of SEQ ID NO: 23; (b) a light chain variable (VL) domain including an amino acid sequence having at least 90% sequence identity to the amino acid sequence of SEQ ID NO: 24; or (c) a VH domain as in (a) and a VL domain as in (b). In some aspects, the anti-PD-L1 antibody includes: (a) a VH domain including an amino acid sequence having at least 95% sequence identity to the amino acid sequence of SEQ ID NO: 23; (b) a VL domain including an amino acid sequence having at least 95% sequence identity to the amino acid sequence of SEQ ID NO: 24; or (c) a VH domain as in (a) and a VL domain as in (b). In some aspects, the anti-PD-L1 antibody includes: (a) a VH domain including an amino acid sequence having at least 95% sequence identity to the amino acid sequence of SEQ ID NO: 23; (b) a VL domain including an amino acid sequence having at least 95% sequence identity to the amino acid sequence of SEQ ID NO: 24; or (c) a VH domain as in (a) and a VL domain as in (b). In some aspects, the anti-PD-L1 antibody includes: (a) a VH domain including an amino acid sequence having at least 96% sequence identity to the amino acid sequence of SEQ ID NO: 23; (b) a VL domain including an amino acid sequence having at least 96% sequence identity to the amino acid sequence of SEQ ID NO: 24; or (c) a VH domain as in (a) and a VL domain as in (b). In some aspects, the anti-PD-L1 antibody includes: (a) a VH domain including an amino acid sequence having at least 97% sequence identity to the amino acid sequence of SEQ ID NO: 23; (b) a VL domain including an amino acid sequence having at least 97% sequence identity to the amino acid sequence of SEQ ID NO: 24; or (c) a VH domain as in (a) and a VL domain as in (b). In some aspects, the anti-PD-L1 antibody includes: (a) a VH domain including an amino acid sequence having at least 98% sequence identity to the amino acid sequence of SEQ ID NO: 23; (b) a VL domain including an amino acid sequence having at least 98% sequence identity to the amino acid sequence of SEQ ID NO: 24; or (c) a VH domain as in (a) and a VL domain as in (b). In some aspects, the anti-PD-L1 antibody includes: (a) a VH domain including an amino acid sequence having at least 99% sequence identity to the amino acid sequence of SEQ ID NO: 23; (b) a VL domain including an amino acid sequence having at least 99% sequence identity to the amino acid sequence of SEQ ID NO: 24; or (c) a VH domain as in (a) and a VL domain as in (b). In some aspects, the anti-PD-L1 antibody includes: (a) a VH domain including the amino acid sequence of SEQ ID NO: 23; (b) a VL domain including the amino acid sequence of SEQ ID NO: 24; or (c) a VH domain as in (a) and a VL domain as in (b). In some aspects, the anti-PD-L1 antibody includes: (a) a VH domain including the amino acid sequence of SEQ ID NO: 23; and (b) a VL domain including the amino acid sequence of SEQ ID NO: 24.

In some aspects, the method includes administering to the individual the PD-L1 axis binding antagonist intravenously. In some aspects, the PD-L1 axis binding antagonist is atezolizumab. In some aspects, atezolizumab is administered to the individual intravenously at a dose of about 840 mg every 2 weeks, about 1200 mg every 3 weeks, or about 1680 mg of every 4 weeks. In some aspects, atezolizumab is administered to the individual intravenously at a dose of about 1200 mg every 3 weeks. In some aspects, atezolizumab is administered to the individual intravenously at a dose of about 1200 mg on Day −2 to Day 4 of one or more 21-day dosing cycles. In some aspects, atezolizumab is administered to the individual intravenously at a dose of about 1200 mg on Day 1 of each 21-day dosing cycle.

In some aspects, the PD-L1 axis binding antagonist is a PD-1 binding antagonist. In some aspects, the PD-1 binding antagonist inhibits the binding of PD-1 to one or more of its ligand binding partners. In some aspects, the PD-1 binding antagonist inhibits the binding of PD-1 to PD-L1, PD-L2, or both PD-L1 and PD-L2.

In some aspects, the PD-1 binding antagonist is an anti-PD-1 antibody. In some aspects, the anti-PD-1 antibody is MDX-1106 (nivolumab), MK-3475 (pembrolizumab), MEDI-0680 (AMP-514), PDR001, REGN2810, or BGB-108.

In some aspects, the PD-1 binding antagonist is an Fc fusion protein. In some aspects, the Fc fusion protein is AMP-224.

In some aspects, the individual is a human.

### DETAILED DESCRIPTION OF THE INVENTION

### I. Introduction

The present invention provides diagnostic and therapeutic methods and compositions for cancer treatment. The invention is based, at least in part, on the discovery that determination of, for example, osteoclast number, CD8⁺ T cell density, and/or activated CD8⁺ T cell number, in samples obtained from an individual having a cancer (e.g., a hematologic cancer, e.g., a myeloma, e.g., a multiple myeloma (MM), e.g., a relapsed or refractory MM) are useful in the diagnosis, treatment, and monitoring of the individual to treatment with an anti-cancer therapy that includes a PD-L1 axis binding antagonist (e.g., an anti-PD-L1 antibody, e.g., atezolizumab) and an anti-CD38 antibody (e.g., an anti-CD38 antagonist antibody, e.g., daratumumab).

### II. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### III. Definitions

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.”

The “amount,” “level,” or “expression level,” used herein interchangeably, of a biomarker is a detectable level in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). Expression levels can be measured by methods known to one skilled in the art and also disclosed herein. The expression level or amount of a biomarker can be used to identify/characterize a subject having a cancer (e.g., a hematologic cancer (e.g., a myeloma (e.g., MM, e.g., a relapsed or refractory MM) or a lymphoma (e.g., a NHL, e.g., a relapsed or refractory DLBCL or a relapsed or refractory FL))) who may be likely to respond to, or benefit from, a particular therapy (e.g., a therapy comprising one or more dosing cycles of a PD-1 axis binding antagonist and an anti-CD38 antibody).

The presence and/or expression level/amount of various biomarkers described herein in a sample can be analyzed by a number of methodologies, many of which are known in the art and understood by the skilled artisan, including, but not limited to, immunohistochemistry (“IHC”), Western blot analysis, immunoprecipitation, molecular binding assays, ELISA, ELIFA, fluorescence activated cell sorting (“FACS”), MassARRAY, proteomics, quantitative blood based assays (e.g., Serum ELISA), biochemical enzymatic activity assays, in situ hybridization, fluorescence in situ hybridization (FISH), Southern analysis, Northern analysis, whole genome sequencing, massively parallel DNA sequencing (e.g., next-generation sequencing), NANOSTRING®, polymerase chain reaction (PCR) including quantitative real time PCR (qRT-PCR) and other amplification type detection methods, such as, for example, branched DNA, SISBA, TMA and the like, RNA-seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

The term “antagonist” is used in the broadest sense, and includes any molecule that partially or fully blocks, inhibits, or neutralizes a biological activity of a native polypeptide disclosed herein. Suitable antagonist molecules specifically include antagonist antibodies or antibody fragments (e.g., antigen-binding fragments), fragments or amino acid sequence variants of native polypeptides, peptides, antisense oligonucleotides, small organic molecules, etc. Methods for identifying antagonists of a polypeptide may comprise contacting a polypeptide with a candidate antagonist molecule and measuring a detectable change in one or more biological activities normally associated with the polypeptide.

“CD38” as used herein, refers to a CD38 glycoprotein found on the surface of many immune cells, including CD4⁺, CD8⁺, B lymphocytes, and natural killer (NK) cells, and includes any native CD38 from any vertebrate source, including mammals such as primates (e.g., humans) and rodents (e.g., mice and rats), unless otherwise indicated. CD38 is expressed at a higher level and more uniformly on myeloma cells as compared to normal lymphoid and myeloid cells. The term encompasses “full-length,” unprocessed CD38, as well as any form of CD38 that results from processing in the cell. The term also encompasses naturally occurring variants of CD38, e.g., splice variants or allelic variants. CD38 is also referred to in the art as cluster of differentiation 38, ADP-ribosyl cyclase 1, cADPr hydrolase 1, and cyclic ADP-ribose hydrolase 1. CD38 is encoded by the CD38 gene. The nucleic acid sequence of an exemplary human CD38 is shown under NCBI Reference Sequence: NM_001775.4 or in SEQ ID NO: 25. The amino acid sequence of an exemplary human CD38 protein encoded by CD38 is shown under UniProt Accession No. P28907 or in SEQ ID NO: 26.

The term “anti-CD38 antibody” encompass all antibodies that bind CD38 with sufficient affinity such that the antibody is useful as a therapeutic agent in targeting a cell expressing the antigen, and do not significantly cross-react with other proteins such as a negative control protein in the assays described below. For example, an anti-CD38 antibody may bind to CD38 on the surface of a MM cell and mediate cell lysis through the activation of complement-dependent cytotoxicity, ADCC, antibody-dependent cellular phagocytosis (ADCP), and apoptosis mediated by Fc cross-linking, leading to the depletion of malignant cells and reduction of the overall cancer burden. An anti-CD38 antibody may also modulate CD38 enzyme activity through inhibition of ribosyl cyclase enzyme activity and stimulation of the cyclic adenosine diphosphate ribose (cADPR) hydrolase activity of CD38. In certain aspects, an anti-CD38 antibody that binds to CD38 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain aspects, the anti-CD38 antibody may bind to both human CD38 and chimpanzee CD38. Anti-CD38 antibodies also include anti-CD38 antagonist antibodies. Bispecific antibodies wherein one arm of the antibody binds CD38 are also contemplated. Also encompassed by this definition of anti-CD38 antibody are functional fragments of the preceding antibodies. Examples of antibodies which bind CD38 include: daratumumab (DARZALEX®) (U.S. Pat. No. 7,829,673 and U.S. Pub. No: 20160067205 A1, expressly incorporated herein by reference); “MOR202” (U.S. Pat. No. 8,263,746, expressly incorporated herein by reference); and isatuximab (SAR-650984) (U.S. Pat. No. 8,153,765, expressly incorporated herein by reference).

The term “PD-L1 axis binding antagonist” refers to a molecule that inhibits the interaction of a PD-L1 axis binding partner with either one or more of its binding partner, so as to remove T-cell dysfunction resulting from signaling on the PD-1 signaling axis, with a result being to restore or enhance T-cell function (e.g., proliferation, cytokine production, and/or target cell killing). As used herein, a PD-L1 axis binding antagonist includes a PD-L1 binding antagonist, a PD-1 binding antagonist, and a PD-L2 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific aspect, an anti-PD-L1 antibody is atezolizumab, marketed as TECENTRIQ® with a WHO Drug Information (International Nonproprietary Names for Pharmaceutical Substances), Proposed INN: List 112, Vol. 28, No. 4, published Jan. 16, 2015 (see page 485) described herein. In another specific aspect, an anti-PD-L1 antibody is MDX-1105 described herein. In still another specific aspect, an anti-PD-L1 antibody is YW243.55.S70. In still another specific aspect, an anti-PD-L1 antibody is MEDI4736 (durvalumab). In still another specific aspect, an anti-PD-L1 antibody is MSB0010718C (avelumab).

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is MDX-1106 (nivolumab) described herein. In another specific aspect, a PD-1 binding antagonist is MK-3475 (pembrolizumab) described herein. In another specific aspect, a PD-1 binding antagonist is MEDI-0680 (AMP-514) described herein. In another specific aspect, a PD-1 binding antagonist is PDR001 described herein. In another specific aspect, a PD-1 binding antagonist is REGN2810 described herein. In another specific aspect, a PD-1 binding antagonist is BGB-108 described herein.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

As used herein, “administering” is meant a method of giving a dosage of a compound (e.g., a PD-L1 axis binding antagonist or an anti-CD38 antibody) or a composition (e.g., a pharmaceutical composition, e.g., a pharmaceutical composition including a PD-L1 axis binding antagonist or an anti-CD38 antibody) to a subject. The compounds and/or compositions utilized in the methods described herein can be administered, for example, intravenously (e.g., by intravenous infusion), subcutaneously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

A “fixed” or “flat” dose of a therapeutic agent (e.g., a PD-L1 axis binding antagonist, e.g., an anti-PD-L1 antagonist antibody, e.g., atezolizumab) herein refers to a dose that is administered to a patient without regard for the weight or body surface area (BSA) of the patient. The fixed or flat dose is therefore not provided as a mg/kg dose or a mg/m² dose, but rather as an absolute amount of the therapeutic agent (e.g., mg).

As used herein, the term “treatment” or “treating” refers to clinical intervention designed to alter the natural course of the individual or cell being treated during the course of clinical pathology. Desirable effects of treatment include delaying or decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “in combination with” or “in conjunction with” refers to administration of one treatment modality in addition to another treatment modality. As such, “in combination with” or “in conjunction with” refers to administration of one treatment modality before, during, or after administration of the other treatment modality to the individual.

A “disorder” or “disease” is any condition that would benefit from treatment including, but not limited to, disorders that are associated with some degree of abnormal cell proliferation, e.g., cancer, e.g., a hematologic cancer, e.g., a myeloma (e.g., multiple myeloma (MM), e.g., a relapsed or refractory MM) or a lymphoma (e.g., a NHL, e.g., a relapsed or refractory diffuse large B cell lymphoma (DLBCL) or a relapsed or refractory follicular lymphoma (FL))).

The term “dysfunction,” in the context of immune dysfunction, refers to a state of reduced immune responsiveness to antigenic stimulation.

The term “dysfunctional,” as used herein, also includes refractory or unresponsive to antigen recognition, specifically, impaired capacity to translate antigen recognition into downstream T-cell effector functions, such as proliferation, cytokine production (e.g., gamma interferon) and/or target cell killing.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include, but are not limited to, hematologic cancers including myeloma and B cell lymphoma (including MM (e.g., relapsed or refractory MM), DLBCL (e.g., relapsed or refractory DLBCL), FL (e.g., relapsed or refractory FL), low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); lung cancer, such as non-small cell lung cancer (NSCLC), which includes squamous NSCLC or non-squamous NSCLC, including locally advanced unresectable NSCLC (e.g., Stage IIIB NSCLC), or recurrent or metastatic NSCLC (e.g., Stage IV NSCLC), adenocarcinoma of the lung, or squamous cell cancer (e.g., epithelial squamous cell cancer); esophageal cancer; cancer of the peritoneum; hepatocellular cancer; gastric or stomach cancer, including gastrointestinal cancer and gastrointestinal stromal cancer; pancreatic cancer; glioblastoma; cervical cancer; ovarian cancer; liver cancer; bladder cancer (e.g., urothelial bladder cancer (UBC), muscle invasive bladder cancer (MIBC), and BCG-refractory non-muscle invasive bladder cancer (NMIBC)); cancer of the urinary tract; hepatoma; breast cancer (e.g., HER2′ breast cancer and triple-negative breast cancer (TNBC), which are estrogen receptors (ER-), progesterone receptors (PR-), and HER2 (HER2-) negative); colon cancer; rectal cancer; colorectal cancer; endometrial or uterine carcinoma; salivary gland carcinoma; kidney or renal cancer (e.g., renal cell carcinoma (RCC)); prostate cancer; vulval cancer; thyroid cancer; hepatic carcinoma; anal carcinoma; penile carcinoma; melanoma, including superficial spreading melanoma, lentigo maligna melanoma, acral lentiginous melanomas, and nodular melanomas; post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage, and tumor clearance.

As used herein, “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

The term “anti-cancer therapy” refers to a therapy useful in treating cancer (e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)). Examples of anti-cancer therapeutic agents include, but are limited to, e.g., immunomodulatory agents (e.g., an immunomodulatory agent (e.g., an agent that decreases or inhibits one or more immune co-inhibitory receptors (e.g., one or more immune co-inhibitory receptors selected from PD-L1, PD-1, CTLA-4, LAG3, TIM3, BTLA, TIGIT, and/or VISTA), such as a CTLA-4 antagonist, e.g., an anti-CTLA-4 antagonist antibody (e.g., ipilimumab (YERVOY®)), an anti-TIGIT antagonist antibody, or an anti-PD-L1 antagonist antibody, or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody)), chemotherapeutic agents, growth inhibitory agents, cytotoxic agents, agents used in radiation therapy, anti-angiogenesis agents, apoptotic agents, anti-tubulin agents, and other agents to treat cancer. Combinations thereof are also included in the invention.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anti-cancer agents disclosed below.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethyl hydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors (e.g., an anaplastic lymphoma kinase (Alk) inhibitor, such as AF-802 (also known as CH-5424802 or alectinib)); (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds described include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; inhibitors of insulin receptor tyrosine kinases, including anaplastic lymphoma kinase (Alk) inhibitors, such as AF-802 (also known as CH-5424802 or alectinib), ASP3026, X396, LDK378, AP26113, crizotinib (XALKORI®), and ceritinib (ZYKADIA®); small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At211, I131, I125, Y90, Re186, Re188, Sm153, Bi212, P32, Pb212 and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g., celecoxib or etoricoxib), proteosome inhibitor (e.g., PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

An “effective amount” of a compound, for example, a PD-L1 axis binding antagonist or an anti-CD38 antibody, or a composition (e.g., pharmaceutical composition) thereof, is at least the minimum amount required to achieve the desired therapeutic result, such as a measurable increase in overall survival or progression-free survival of a particular disease or disorder (e.g., cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL). An effective amount herein may vary according to factors such as the disease state, age, sex, and weight of the patient, and the ability of the antibody to elicit a desired response in the subject. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease (e.g., reduction or delay in cancer-related pain, increasing the quality of life of those suffering from the disease, decreasing the dose of other medications required to treat the disease, enhancing effect of another medication such as via targeting, delaying the progression of the disease (e.g., progression-free survival); delay of unequivocal clinical progression (e.g., cancer-related pain progression, deterioration in Eastern Cooperative Group Oncology Group (ECOG) Performance Status (PS) (e.g., how the disease affects the daily living abilities of the patient), and/or initiation of next systemic anti-cancer therapy), and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

“Immunogenicity” refers to the ability of a particular substance to provoke an immune response. Tumors are immunogenic and enhancing tumor immunogenicity aids in the clearance of the tumor cells by the immune response. Examples of enhancing tumor immunogenicity include, but are not limited to, treatment with an anti-PD-L1 antibody and an anti-CD38 antibody.

“Individual response” or “response” can be assessed using any endpoint indicating a benefit to the subject, including, without limitation, (1) inhibition, to some extent, of disease progression (e.g., progression of cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e. reduction, slowing down or complete stopping) of metastasis; (5) relief, to some extent, of one or more symptoms associated with the disease or disorder (e.g., cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)); (6) increase or extend in the length of survival, including overall survival and progression-free survival; and/or (9) decreased mortality at a given point of time following treatment.

An “objective response” refers to a measurable response including complete response (CR) or partial response (PR). In some aspects, “objective response rate” (ORR) refers to the sum of complete response (CR) rate and partial response (PR) rate. For MM, ORR may be defined as the proportion of patients with best overall response of stringent complete response (sCR), complete response (CR), very good partial response (VGPR), or partial response (PR) (see, e.g., Table 1, below), as defined by the International Myeloma Working Group Uniform Response (IMWG) criteria, as disclosed in Durie et al. Leukemia. 20(9):1467-73 (2006), Durie et al. Leukemia. 29:2416-7 (2015), and Kumar et al. Lancet Oncol. 17:e328-46 (2016), which are incorporated herein by reference in their entireties.

As used herein, “duration of objective response” (DOR) is defined as the time from the first occurrence of a documented objective response to disease progression (e.g., according to IMWG criteria for MM (see, e.g., Tables 2 and 3, below), or death from any cause within 30 days of the last dose of a treatment, whichever occurs first.

As used herein, “survival” refers to the patient remaining alive, and includes overall survival as well as progression-free survival.

As used herein, “overall survival” (OS) refers to the percentage of subjects in a group who are alive after a particular duration of time, e.g., 1 year or 5 years from the time of diagnosis or treatment. In some aspects, OS may be defined as the time from enrollment to death from any cause.

As used herein, “progression-free survival” (PFS) refers to the length of time during and after treatment during which the disease being treated (e.g., cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)) does not get worse, i.e., does not progress (e.g., according to IMWG criteria for MM (see, e.g., Tables 2 and 3, below). Progression-free survival may include the amount of time patients have experienced a complete response or a partial response, as well as the amount of time patients have experienced stable disease. As the skilled person will appreciate, a patients' progression-free survival is improved or enhanced if the patient experiences a longer length of time during which the disease does not progress as compared to the average or mean progression-free survival time of a control group of similarly situated patients.

As used herein, “complete response” or “CR” refers to disappearance of all signs of cancer (e.g., disappearance of target lesions). This does not always mean the cancer has been cured. For MM, CR is further defined according to the IMWG criteria (e.g., as described in Table 1, below).

As used herein, “stringent complete response” or “sCR” refers to a complete response as defined by the IMWG criteria (e.g., as described in Table 1, below) plus normal free light chain (FLC) ratio and absence of clonal cells in bone marrow by immunohistochemistry (kappa/lambda ratio≤4:1 or ≥1:2 for kappa and lambda patients, respectively after counting 100 plasma cells).

As used herein, “partial response” or “PR” refers to a decrease in the size of one or more lesions or tumors, or in the extent of cancer in the body, in response to treatment. With respect to MM, PR refers to at least a 50% reduction of serum M-protein and at least a 90% reduction in 24 hr urinary M-protein or to a level of less than 200 mg/24 hr. For MM, PR is further defined according to the IMWG criteria (e.g., as described in Table 1, below).

As used herein, “very good partial response” or “VG PR” refers to serum and urine M-protein detectable by immunofixation but not on electrophoresis; or ≥90% reduction in serum M-protein-plus urine M-protein level<100 mg/24 hr, as defined by the IMGW criteria (see, e.g., Table 1, below).

As used herein, “minimal response” or “MR” is defined per the IMGW criteria (see, e.g., Table 2, below) and refers to ≥25% but ≤49% reductions of serum M-protein and reduction in 24-hour urine M-protein by 50%-89%, and additionally, if present at baseline, 25%-49% reduction in the size (SPD)^{c} of soft tissue plasmacytomas.

As used herein, “stable disease” or “SD” refers to neither sufficient shrinkage of target lesions and/or a decrease in the extent of cancer in the body to qualify for PR, nor sufficient increase to qualify for PD. For MM, SD refers to a response otherwise not meeting the criteria for MR, CR, VGPR, PR, or PD as defined according to the IMWG criteria (e.g., as described in Tables 1 and 2, below).

As used herein, “progressive disease” or “PD” refers to an increase in the size of one or more lesions or tumors, or in the extent of cancer in the body, in response to treatment. PD, with respect to MM, refers to an increase of at least 25% from the lowest response value in at least one of the following: (a) serum M-protein, (b) urine M-protein, (c) the difference between involved and uninvolved FLC levels, (d) bone marrow plasma cell percentage irrespective of baseline status, (e) the appearance of new lesion(s), or (f) at least a 50% increase in circulating plasma cells. For MM, PD is further defined according to the IMWG criteria (e.g., as described in Table 2, below).

“Clinical relapse,” as used herein refers to direct indications of increasing disease and/or end organ dysfunction relating to the underlying clonal plasma cell proliferative disorder. For MM, clinical relapse is defined according to the IMWG criterial (see, e.g., Table 2, below) and includes one or more of (a) development of new soft tissue plasmacytomas or bone lesions, (b) definite increase in the size of existing plasmacytomas or bone lesions, defined as a 50% (and ≥1 cm) increase as measured serially by the sum of the products of the cross-diameters of the measurable lesion, (c) hypercalcemia>11 mg/dL (2.65 mm/L), (d) decrease in in hemoglobin of ≥2 g/dL (1.25 mmol/L) not related to therapy or other non-myeloma related conditions, (e) a rise in serum creatinine by 2 mg/dL or more (177 μmol/L or more) from the start of therapy and attributable to myeloma, and/or (f) hyperviscosity related to serum paraprotein.

As used herein, “delaying progression” of a disorder or disease means to defer, hinder, slow, retard, stabilize, and/or postpone development of the disease or disorder (e.g., cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)). This delay can be of varying lengths of time, depending on the history of the disease and/or subject being treated. As is evident to one skilled in the art, a sufficient or significant delay can, in effect, encompass prevention, in that the subject does not develop the disease. For example, in a late stage cancer, development of central nervous system (CNS) metastasis, may be delayed.

As used herein, the term “reducing or inhibiting cancer relapse” means to reduce or inhibit tumor or cancer relapse, or tumor or cancer progression.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated (e.g., cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)), the presence or size of metastases, or the size of the primary tumor.

As used herein, “reference osteoclast number” is a baseline number of osteoclasts in a reference population of individuals having a hematologic cancer, wherein the reference population consists of individuals who are treated with a PD-L1 axis binding antagonist and an anti-CD38 antibody, and whereby the reference osteoclast number significantly separates subsets of individuals in the reference population based on a significant difference in responsiveness to treatment with the PD-L1 axis binding antagonist and the anti-CD38 antibody. In some instances, the reference osteoclast number may be pre-assigned.

As used herein, “reference CD8⁺ T cell density” is a baseline CD8⁺ T cell density of CD8⁺ T cells within tumor clusters in a reference population of individuals having a hematologic cancer, wherein the reference population consists of individuals who are treated with a PD-1 axis binding antagonist and an anti-CD38 antibody, and whereby the reference CD8⁺ T cell density significantly separates subsets of individuals in the reference population based on a significant difference in responsiveness to treatment with the PD-L1 axis binding antagonist and the anti-CD38 antibody. In some instances, the reference CD8⁺ T cell density may be pre-assigned.

As used herein, “reference number of activated CD8⁺ T cells” is the number of CD8⁺HLA-DR+Ki-67⁺ T cells in a biological sample (e.g., bone marrow or blood) from an individual with a hematologic cancer obtained prior to administration of a PD-L1 axis binding antagonist and an anti-CD38 antibody; at a previous time point, wherein the previous time point is following administration of a PD-L1 axis binding antagonist and an anti-CD38 antibody, but prior to further administration of the PD-L1 axis binding antagonist and anti-CD38 antibody, wherein the reference number of activated CD8⁺ T cells significantly separates subsets of individuals in a reference population based on a significant difference in responsiveness to treatment with the PD-L1 axis binding antagonist and the anti-CD38 antibody. In some instances, the reference number of activated CD8⁺ T cells may be a pre-assigned number.

By “extending survival” is meant increasing overall or progression-free survival in a treated patient relative to an untreated patient (e.g., relative to a patient not treated with the medicament), or relative to a patient who does not express a biomarker at the designated level, and/or relative to a patient treated with an approved anti-tumor agent. An objective response refers to a measurable response, including stringent complete response (sCR), complete response (CR), very good partial response (VGPR), partial response (PR), and minimal response (MR).

The terms “detecting” and “detection” are used herein in the broadest sense to include both qualitative and quantitative measurements of a target molecule. Detecting includes identifying the mere presence of the target molecule in a sample as well as determining whether the target molecule is present in the sample at detectable levels. Detecting may be direct or indirect.

The term “biomarker” as used herein refers to an indicator, e.g., predictive, diagnostic, and/or prognostic, which can be detected in a sample. The biomarker may serve as an indicator of a particular subtype of a disease or disorder (e.g., cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)) characterized by certain, molecular, pathological, histological, and/or clinical features. In some aspects, a biomarker is a gene. Biomarkers include, but are not limited to, polypeptides, polynucleotides (e.g., DNA, and/or RNA), polynucleotide copy number alterations (e.g., DNA copy numbers), polypeptide and polynucleotide modifications (e.g., posttranslational modifications), carbohydrates, and/or glycolipid-based molecular markers.

The term “antibody” includes monoclonal antibodies (including full-length antibodies which have an immunoglobulin Fc region), antibody compositions with polyepitopic specificity, multispecific antibodies (e.g., bispecific antibodies), diabodies, and single-chain molecules, as well as antibody fragments, including antigen-binding fragments, such as Fab, F(ab′)₂, and Fv. The term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the CL is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in the CH sequence and function, e.g., humans express the following subclasses: IgG1, IgG2A, IgG2B, IgG3, IgG4, IgA1 and IgA2.

The term “hypervariable region” or “HVR” as used herein refers to each of the regions of an antibody variable domain which are hypervariable in sequence (“complementarity determining regions” or “CDRs”). Generally, antibodies comprise six CDRs: three in the VH (CDR-H1, CDR-H2, CDR-H3), and three in the VL (CDR-L1, CDR-L2, CDR-L3). Exemplary CDRs herein include:

(a) CDRs occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917, 1987);

(b) CDRs occurring at amino acid residues 24-34 (L1), 50-56 (L2), 89-97 (L3), 31-35b (H1), 50-65 (H2), and 95-102 (H3) (Kabat et al. Sequences of Proteins of Immunological Interest, 5th Ed. Public Health Service, National Institutes of Health, Bethesda, Md. (1991)); and

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745, 1996).

Unless otherwise indicated, HVR residues and other residues in the variable domain (e.g., FR residues) are numbered herein according to Kabat et al. supra.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et aL, Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full-length antibody,” “intact antibody,” and “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen-binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 (1995)); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Functional fragments” of the antibodies comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described include human IgG1, IgG2 (IgG2A, IgG2B), IgG3 and IgG4. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

“Affinity” refers to the strength of the sum total of non-covalent interactions between a single binding site of a molecule (e.g., an antibody) and its binding partner (e.g., an antigen, e.g., PD-L1 or CD38). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary aspects for measuring binding affinity are described in the following.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See, e.g., M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Humanized” forms of non-human (e.g., murine) antibodies are chimeric antibodies that contain minimal sequence derived from non-human immunoglobulin. In one aspect, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some aspects, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

The term “isolated antibody” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al., J. Chromatogr. B 848:79-87 (2007). In preferred aspects, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one aspect, the extent of binding of an antibody to an unrelated target is less than about 10% of the binding of the antibody to the target as measured, for example, by a radioimmunoassay (RIA). In certain aspects, an antibody that specifically binds to a target has a dissociation constant (K_{D}) of ≤1 μM, ≤100 nM, ≤10 nM, ≤1 nM, or ≤0.1 nM. In certain aspects, an antibody specifically binds to an epitope on a protein that is conserved among the protein from different species. In another aspect, specific binding can include, but does not require exclusive binding. The term as used herein can be exhibited, for example, by a molecule having a K_{D} for the target of 10⁻⁴M or lower, alternatively 10⁻⁵M or lower, alternatively 10⁻⁶ M or lower, alternatively 10⁻⁷ M or lower, alternatively 10⁻⁸ M or lower, alternatively 10⁻⁰ M or lower, alternatively 10⁻¹⁰ M or lower, alternatively 10⁻¹¹ M or lower, alternatively 10⁻¹² M or lower or a K_{D} in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and K_{D} values are inversely related. A high affinity for an antigen is measured by a low K_{D} value. In one aspect, the term “specific binding” refers to binding where a molecule binds to a particular polypeptide or epitope on a particular polypeptide without substantially binding to any other polypeptide or polypeptide epitope.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for aspect, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, “subject” or “individual” is meant a mammal, including, but not limited to, a human or non-human mammal, such as a bovine, equine, canine, ovine, or feline. In some aspects, the subject is a human. Patients are also subjects herein.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “tumor sample,” “disease sample,” and variations thereof refers to any sample obtained from a subject of interest that would be expected or is known to contain the cellular and/or molecular entity that is to be characterized. In some aspects, the sample is a tumor tissue sample (e.g., a tumor biopsy, e.g., a lymph node biopsy (e.g., lymph fluid)), a bone marrow sample (e.g., a bone marrow aspirate), or a blood sample (e.g., a whole blood sample, a serum sample, or a plasma sample). Other samples include, but are not limited to, primary or cultured cells or cell lines, cell supernatants, cell lysates, platelets, vitreous fluid, synovial fluid, follicular fluid, seminal fluid, amniotic fluid, milk, blood-derived cells, urine, cerebro-spinal fluid, saliva, sputum, tears, perspiration, mucus, stool, tumor lysates, and tissue culture medium, tissue extracts such as homogenized tissue, cellular extracts, and combinations thereof.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for aspect, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The terms “polynucleotide” and “nucleic acid” specifically includes mRNA and cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, aspects wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The phrase “pharmaceutically acceptable” indicates that the substance or composition must be compatible chemically and/or toxicologically, with the other ingredients comprising a formulation, and/or the mammal being treated therewith.

The term “pharmaceutical formulation” refers to a preparation which is in such form as to permit the biological activity of an active ingredient contained therein to be effective, and which contains no additional components which are unacceptably toxic to a subject to which the formulation would be administered.

An “article of manufacture” is any manufacture (e.g., a package or container) or kit comprising at least one reagent, e.g., a medicament for treatment of a disease or disorder (e.g., cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)), and a package insert. In certain aspects, the manufacture or kit is promoted, distributed, or sold as a unit for performing the methods described herein.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments.

### IV. Diagnostic Methods and Uses

Provided herein are diagnostic methods and uses for treating cancer (e.g., a hematologic cancer, e.g., a myeloma (e.g., a multiple myeloma (MM), e.g., a relapsed or refractory MM)) in an individual who may benefit from a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab).

Osteoclast Number as a Predictive Biomarker

The invention is based, at least in part, on the discovery that the number of osteoclasts present in a tumor sample obtained from an individual with a hematologic cancer (e.g., myeloma, e.g., multiple myeloma (MM), e.g., a relapsed or refractory MM) can be used to identify the individual as one who may benefit from a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab). In particular, an individual having a hematologic cancer (e.g., myeloma, e.g., multiple myeloma (MM), e.g., a relapsed or refractory MM) may be identified as likely to benefit from a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab) based on an osteoclast number that is lower than a reference osteoclast number. Accordingly, the invention features a method of identifying an individual having a hematologic cancer (e.g., myeloma, e.g., multiple myeloma (MM), e.g., a relapsed or refractory MM) who may benefit from a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab), the method including determining an osteoclast number in a tumor sample obtained from the individual, wherein an osteoclast number that is lower than a reference osteoclast number identifies the individual as one who may benefit from the treatment.

In some instances, the osteoclast number in the tumor sample is the number of osteoclasts within a tumor region. In certain embodiments, the tumor region contains an area containing tumor cells and adjacent myeloid cells. In some instances, the tumor region does not contain fat bodies and bone trabeculae. In some embodiments, the tumor region contains an area within about 40 μm to about 1 mm (e.g., between about 40 μm to about 900 μm, e.g., between about 40 μm to about 850 μm, e.g., between about 40 μm to about 700 μm, e.g., between about 40 μm to about 600 μm, e.g., between about 40 μm to about 500 μm, e.g., between about 40 μm to about 400 μm, e.g., between about 40 μm to about 350 μm, e.g., between about 40 μm to about 300 μm, e.g., between about 50 μm to about 300 μm, e.g., between about 60 μm to about 300 μm, e.g., between about 70 μm to about 300 μm, e.g., between about 80 μm to about 300 μm, e.g., between about 90 μm to about 300 μm, e.g., between about 100 μm to about 300 μm, e.g., between about 100 μm to about 280 μm, e.g., between about 100 μm to about 260 μm, e.g., between about 100 μm to about 240 μm, e.g., between about 100 μm to about 220 μm, e.g., between about 100 μm to about 200 μm, e.g., between about 110 μm to about 200 μm, e.g., between about 120 μm to about 200 μm, e.g., between about 130 μm to about 200 μm, e.g., between about 140 μm to about 200 μm, e.g., between about 150 μm to about 200 μm, e.g., between about 160 μm to about 200 μm, e.g., between about 170 μm to about 200 μm, e.g., between about 180 μm to about 200 μm, e.g., between about 190 μm to about 200 μm, e.g., 190, 191, 192, 193, 194, 195, 196, 197, 198, 199, or 200 μm), such as about 200 μm of a tumor cell or a myeloid cell adjacent to a tumor cell. In some embodiments, the tumor region contains an area within 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 200, 210, 220, 230, 240, 250, 260, 270, 280, 290, 300, 320, 340, 350, 360, 380, 400, 450, 500, 550, 600, 700, 800, 900, or 1000 μm of a tumor cell or a myeloid cell adjacent to a tumor cell.

In some embodiments, when the osteoclast number in the tumor sample is lower than the reference osteoclast number, the individual may be administered a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab).

In some instances, reference osteoclast number is a pre-assigned number of osteoclasts in a reference population of individuals having the hematologic cancer, the reference population consisting of individuals who have been treated with a PD-L1 axis binding antagonist and an anti-CD38 antibody. In some aspects, the reference osteoclast number significantly separates subsets of individuals in the reference population based on a significant difference in responsiveness to treatment with the PD-L1 axis binding antagonist and the anti-CD38 antibody. The reference osteoclast number may be between 1 and about 200 osteoclast cells (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 95, 100, 101, 102, 103, 104, 105, 110, 115, 120, 130, 140, 150, 160, 170, 180, 190, or 200 osteoclast cells). Preferably, the reference osteoclast number may be between about 3 and about 70 osteoclast cells (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 osteoclast cells).

In some embodiments, tumor samples (e.g., a biopsy) may be taken from the individual prior to the initiation of treatment a PD-L1 axis binding antagonist and an anti-CD38 antibody, such as, between about 3 days to about 20 weeks (e.g., 3 days, 4 days, 5 days, 6 days, 1 week, 2 weeks, 4 weeks, 8 weeks, 12 weeks, 16 weeks, or 20 weeks), such as about 4 weeks before initiation of treatment.

CD8⁺ T Cell Density as a Predictive Biomarker

The invention is based, at least in part, on the discovery that the density of CD8⁺ T cells present in a tumor sample obtained from an individual with a hematologic cancer (e.g., myeloma, e.g., multiple myeloma (MM), e.g., a relapsed or refractory MM) can be used to identify the individual as one who may benefit from a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab). In particular, an individual having a hematologic cancer (e.g., myeloma, e.g., multiple myeloma (MM), e.g., a relapsed or refractory MM) may be identified as likely to benefit from a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab) based on a CD8⁺ T cell density that is higher than a reference CD8⁺ T cell density. Accordingly, the invention features a method of identifying an individual having a hematologic cancer (e.g., myeloma, e.g., multiple myeloma (MM), e.g., a relapsed or refractory MM) who may benefit from a treatment including a PD-L1 axis binding antagonist (e.g., an anti-PD-L1 antibody, e.g., atezolizumab) and an anti-CD38 antibody (e.g., an anti-CD38 antagonist antibody (e.g., daratumumab)), the method including determining a CD8⁺ T cell density in a tumor sample obtained from the individual, wherein a CD8⁺ T cell density that is higher than a reference CD8⁺ T cell density identifies the individual as one who is more likely to benefit from the treatment.

In some instances, the CD8⁺ T cell density in the tumor sample is the density of CD8⁺ T cells within a tumor cluster. In some embodiments, the tumor cluster is an area containing adjacent tumor cells. In some embodiments, the tumor cluster is at least about 25 μm to about 400 μm (e.g., between about 25 μm to about 380 μm, e.g., between about 25 μm to about 360 μm, e.g., between about 25 μm to about 340 μm, e.g., between about 25 μm to about 320 μm, e.g., between about 25 μm to about 300 μm, e.g., between about 25 μm to about 280 μm, e.g., between about 25 μm to about 260 μm, e.g., between about 25 μm to about 240 μm, e.g., between about 25 μm to about 220 μm, e.g., between about 25 μm to about 200 μm, e.g., between about 25 μm to about 180 μm, e.g., between about 25 μm to about 160 μm, e.g., between about 25 μm to about 140 μm, e.g., between about 25 μm to about 120 μm, e.g., between about 25 μm to about 100 μm, e.g., between about 25 μm to about 90 μm, e.g., between about 25 μm to about 80 μm, e.g., between about 25 μm to about 75 μm, e.g., between about 30 μm to about 70 μm, e.g., between about 35 μm to about 65 μm, e.g., between about 40 μm to about 60 μm, e.g., between about 45 μm to about 55 μm, e.g., 45, 46, 47, 48, 49, 50, 51, 52, 53, 54, or 55 μm), such as about 50 μm, in length along its longest axis. In some embodiments, the tumor cluster is 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, or 400 μm in length along its longest axis. In some embodiments, the tumor cluster is a tumor cell mass with an area of at least about 500 μm² to about 125000 μm² (e.g., between about 500 μm² to about 120000 μm², e.g., between about 500 μm² to about 110000 μm², e.g., between about 500 μm² to about 100000 μm², e.g., between about 500 μm² to about 90000 μm², e.g., between about 500 μm² to about 80000 μm², e.g., between about 500 μm² to about 70000 μm², e.g., between about 500 μm² to about 60000 μm², e.g., between about 500 μm² to about 50000 μm², e.g., between about 500 μm² to about 45000 μm², e.g., between about 500 μm² to about 40000 μm², e.g., between about 500 μm² to about 35000 μm², e.g., between about 500 μm² to about 30000 μm², e.g., between about 500 μm² to about 25000 μm², e.g., between about 500 μm² to about 20000 μm², e.g., between about 500 μm² to about 15000 μm², e.g., between about 500 μm² to about 10000 μm², e.g., between about 500 μm² to about 9000 μm², e.g., between about 500 μm² to about 8000 μm², e.g., between about 500 μm² to about 6000 μm², e.g., between about 500 μm² to about 5000 μm², e.g., between about 700 μm² to about 4000 μm², e.g., between about 1000 μm² to about 3500 μm², e.g., between about 1250 μm² to about 3000 μm², e.g., between about 1500 μm² to about 2500 μm², e.g., between about 1750 μm² to about 2250 μm², e.g., between about 1800 μm² to about 2200 μm², e.g., between about 1850 μm² to about 2150 μm², e.g., between about 1900 μm² to about 2100 μm², e.g., between about 1950 μm² to about 2050 μm², e.g., 1950, 1960, 1970, 1980, 1990, 2000, 2010, 2020, 2030, 2040, or 2050 μm²), such as about 2000 μm². In some embodiments, the tumor cluster is a tumor cell mass with an area of 500, 600, 700, 800, 900, 1000, 1100, 1200, 1300, 1400, 1450, 1500, 1550, 1600, 1650, 1700, 1750, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2250, 2300, 2350, 2400, 2450, 2500, 2600, 2700, 2800, 2900, 3000, 3500, 4000, 4500, 5000, 6000, 7000, 8000, 9000, 10000, 15000, 20000, 25000, 30000, 35000, 40000, 45000, 50000, 55000, 60000, 65000, 70000, 80000, 90000, 100000, 110000, or 120000 μm².

In some embodiments, when the CD8⁺ T cell density in the tumor sample is higher than the reference CD8⁺ T cell density, the individual may be administered a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab).

In some instances, reference CD8⁺ T cell density is a pre-assigned CD8⁺ T cell density of CD8⁺ T cells within tumor clusters in a reference population of individuals having the hematologic cancer, the reference population consisting of individuals who have been treated with a PD-1 axis binding antagonist and an anti-CD38 antibody. In some aspects, the reference CD8⁺ T cell density significantly separates subsets of individuals in the reference population based on a significant difference in responsiveness to treatment with the PD-L1 axis binding antagonist and the anti-CD38 antibody. The reference CD8⁺ T cell density may be between about 100 and about 700 objects/mm² area (e.g., 100, 101, 102, 103, 104, 105, 106, 107, 108, 109, 110, 115, 120, 130, 140, 150, 175, 200, 225, 250, 300, 400, 500, 600, or 700 objects/mm² area). Preferably, the reference CD8⁺ T cell density may be between about 200 and 600 objects/mm² area (e.g., 200, 201, 202, 203, 204, 205, 206, 207, 208, 209, 210, 215, 220, 225, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 objects/mm² area).

In some embodiments, tumor samples (e.g., a biopsy) may be taken from the individual prior to the initiation of treatment a PD-L1 axis binding antagonist and an anti-CD38 antibody, such as, between about 3 days to about 20 weeks (e.g., 3 days, 4 days, 5 days, 6 days, 1 week, 2 weeks, 4 weeks, 8 weeks, 12 weeks, 16 weeks, or 20 weeks), such as about 4 weeks before initiation of treatment.

Use of Activated CD8÷ T Cell Number to Monitor Treatment Responsiveness

The invention is based, at least in part, on the discovery that the number of activated CD8⁺ T cells (CD8⁺HLA-DR⁺Ki-67⁺ T cells) in the bone marrow can be used to monitor responsiveness of an individual having a hematologic cancer (e.g., a myeloma (e.g., a multiple myeloma (MM), e.g., a relapsed or refractory MM)) to a treatment including a PD-1 axis binding antagonist (e.g., an anti-PD-L1 antibody, e.g., atezolizumab) and an anti-CD38 antibody (e.g., an anti-CD38 antagonist antibody, e.g., daratumumab). In particular, an individual having a hematologic cancer (e.g., myeloma, e.g., multiple myeloma (MM), e.g., a relapsed or refractory MM) may be monitored for responsiveness to a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab) based on an increase in the number of activated CD8⁺ T cells. Accordingly, the method includes (a) determining the number of activated CD8⁺ T cells in the bone marrow using a biological sample from the individual at a time point following administration of the PD-1 axis binding antagonist and the anti-CD38 antibody; and (b) comparing the number of activated CD8⁺ T cells in the biological sample to a reference number of activated CD8⁺ T cells, wherein an increase in the number of activated CD8⁺ T cells in the biological sample relative to the reference number of activated CD8⁺ T cells indicates that the individual is responding to the treatment.

In some instances, the number of activated CD8⁺ T cells in the biological sample is increased relative to the reference number of activated CD8⁺ T cells.

In some embodiments, the method includes administering a further dose of the PD-L1 axis binding antagonist and the anti-CD38 antibody to the individual based on the increase in the number of activated CD8⁺ T cells in the biological sample determined in step (b).

In some embodiments, the reference number of activated CD8⁺ T cells is the number of activated CD8⁺ T cells in a biological sample from the individual obtained prior to administration of the PD-L1 axis binding antagonist and the anti-CD38 antibody. In some aspects, the reference number of activated CD8⁺ T cells is the number of activated CD8⁺ T cells in a biological sample is obtained from the individual at a previous time point, wherein the previous time point is following administration of the PD-L1 axis binding antagonist and the anti-CD38 antibody. In some instances, the reference number of activated CD8⁺ T cells is a pre-assigned number of activated CD8⁺ T cells.

In some embodiments, reference number of activated CD8⁺ T cells can be the number of activated T cells in a biological sample (e.g., bone marrow or blood) from the individual between about 1 minute to about 12 months (e.g., 1 minute, 5 minutes, 10 minutes, 20 minutes, 30 minutes, 40 minutes, 50 minutes, 1 hour, 2 hours, 3 hours, 4 hours, 5 hours, 6 hours, 8 hours, 10 hours, 12 hours, 16 hours, 20 hours, 1 day, 2 days, 3 days, 4 days, 5 days, 6 days, 1 week, 2 weeks, 4 weeks, 8 weeks, 12 weeks, 4 months, 5 months, 6 months, 8 months, 10 months, or 12 months), such as about 2 weeks, prior to administration of the PD-L1 axis binding antagonist and the anti-CD38 antibody.

In some aspects, reference number of activated CD8⁺ T cells can be the number of activated T cells in a biological sample obtained from the individual at a previous time point, wherein the previous time point is following administration of the PD-L1 axis binding antagonist and the anti-CD38 antibody. The previous time point can be about 1 minute to about 12 months (e.g., 1 minute, 5 minutes, 10 minutes, 20 minutes, 30 minutes, 40 minutes, 50 minutes, 1 hour, 2 hours, 3 hours, 4 hours, 5 hours, 6 hours, 8 hours, 10 hours, 12 hours, 16 hours, 20 hours, 1 day, 2 days, 3 days, 4 days, 5 days, 6 days, 1 week, 2 weeks, 4 weeks, 8 weeks, 12 weeks, 4 months, 5 months, 6 months, 8 months, 10 months, or 12 months), such as about 2 weeks, following administration of the PD-L1 axis binding antagonist and the anti-CD38 antibody. The previous time point can be about 1 week to about 12 months (e.g., 1 week, 2 weeks, 4 weeks, 8 weeks, 12 weeks, 4 months, 5 months, 6 months, 8 months, 10 months, or 12 months) prior to the subsequent time point

In some aspects, reference number of activated CD8⁺ T cells can be a pre-assigned number. The pre-assigned reference number of activated CD8⁺ T cells may be between about 1×10⁵ and about 1×10⁸ cells (e.g., between about 1×10⁵ and about 1×10⁸ cells, e.g., between about 2×10⁵ and about 9×10⁷ cells, e.g., between about 3×10⁵ and about 8×10⁷ cells, e.g., between about 4×10⁵ and about 7×10⁷ cells, e.g., between about 5×10⁵ and about 6×10⁷ cells, e.g., between about 6×10⁵ and about 5×10⁷ cells, e.g., between about 7×10⁵ and about 4×10⁷ cells, e.g., between about 8×10⁵ and about 3×10⁷ cells, e.g., between about 9×10⁵ and about 2×10⁷ cells, e.g., between about 1×10⁶ and about 1×10⁷ cells, e.g., between about 1×10⁶ and about 9×10⁶ cells, e.g., 1×10⁵, 1.1×10⁵, 1.2×10⁵, 1.3×10⁵, 1.4×10⁵, 1.5×10⁵, 1.6×10⁵, 1.7×10⁵, 1.8×10⁵, 1.9×10⁵, 2×10⁵, 2.5×10⁵, 3×10⁵, 3.5×10⁵, 4×10⁵, 4.5×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, or 1×10⁸ cells). In some embodiments, the pre-assigned reference number of activated CD8⁺ T cells may be 1×10⁵, 1.1×10⁵, 1.2×10⁵, 1.3×10⁵, 1.4×10⁵, 1.5×10⁵, 1.6×10⁵, 1.7×10⁵, 1.8×10⁵, 1.9×10⁵, 2×10⁵, 2.5×10⁵, 3×10⁵, 3.5×10⁵, 4×10⁵, 4.5×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, or 1×10⁸ cells.

In some embodiments, an increase between at least about 1.1- and about 100-fold (e.g., 1.1-, 1.15-, 1.2-, 1.3-, 1.4-, 1.5-, 1.75-, 2-, 3-, 4-, 5-, 6-, 7-, 8-, 9-, 10-, 11-, 12-, 13-, 14-, 15-, 16- , 17-, 18-, 19-, 20-, 21-, 22-, 23-, 24-, 25-, 26-, 27-, 28-, 29-, 30-, 35-, 40-, 45-, 50-, 60-, 70-, 80-, 90-, or 100-fold), such as about 2-fold, in the number of activated CD8⁺ T cells in the biological sample compared to the reference number of activated CD8⁺ T cells identifies the individual as responding to the treatment.

In some aspects, the biological sample is bone marrow aspirate.

In some aspects, the biological sample is blood.

### V. Therapeutic Methods and Uses

The present invention provides methods for treating an individual having a hematologic cancer (e.g., a myeloma (e.g., a multiple myeloma (MM), e.g., a relapsed or refractory MM)). In some instances, the methods of the invention include administering to the patient a PD-L1 axis binding antagonist (e.g., an anti-PD-L1 antibody, e.g., atezolizumab) and an anti-CD38 antibody (e.g., an anti-CD38 antagonist antibody, e.g., daratumumab) based on the biomarkers of the disclosure (e.g., osteoclast number, CD8⁺ T cell density, or number of activated CD8⁺ T cells). Any of the PD-L1 axis binding antagonists, anti-CD38 antibodies, or other anti-cancer agents described herein or known in the art may be used in the methods.

Osteoclast Number as a Predictive Biomarker for Therapeutic Methods

The invention is based, at least in part, on the discovery that the number of osteoclasts present in a tumor sample obtained from an individual with a hematologic cancer (e.g., myeloma, e.g., multiple myeloma (MM), e.g., a relapsed or refractory MM) can be used to identify the individual as one who may benefit from a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab). In particular, an individual having a hematologic cancer (e.g., myeloma, e.g., multiple myeloma (MM), e.g., a relapsed or refractory MM) may be identified as likely to benefit from a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab) based on an osteoclast number that is lower than a reference osteoclast number.

Accordingly, the invention features a method of treating an individual having a hematologic cancer (e.g., myeloma, e.g., multiple myeloma (MM), e.g., a relapsed or refractory MM) who may benefit from a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab), the method including determining an osteoclast number in a tumor sample obtained from the individual, wherein an osteoclast number that is lower than a reference osteoclast number identifies the individual as one who may benefit from the treatment.

In some instances, an osteoclast number in a tumor sample obtained from the individual is lower (e.g., at least by between about 1 to about 50 osteoclast cells (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 osteoclast cells)) than a reference osteoclast number, the individual may be administered a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab).

In some embodiments, the method includes treating an individual having a hematologic cancer (e.g., myeloma, e.g., multiple myeloma (MM), e.g., a relapsed or refractory MM), the method including: (a) determining an osteoclast number in a tumor sample (e.g., a tumor biopsy) obtained from the individual, wherein the osteoclast number in the tumor sample has been determined to be lower (e.g., at least by between about 1 to about 50 osteoclast cells (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 osteoclast cells)) than a reference osteoclast number; and (b) administering an effective amount of a PD-L1 axis binding antagonist (e.g., an anti-PD-L1 antibody, e.g., atezolizumab) and an anti-CD38 antibody (e.g., an anti-CD38 antagonist antibody, e.g., daratumumab) to the individual based on the osteoclast number in the tumor sample determined in step (a).

In some instances, the method of treating an individual having a hematologic cancer includes administering to the individual an effective amount of a PD-L1 axis binding antagonist (e.g., an anti-PD-L1 antibody, e.g., atezolizumab) and an anti-CD38 antibody (e.g., an anti-CD38 antagonist antibody, e.g., daratumumab), wherein prior to treatment, such as, between about 3 days to about 20 weeks (e.g., 3 days, 4 days, 5 days, 6 days, 1 week, 2 weeks, 4 weeks, 8 weeks, 12 weeks, 16 weeks, or 20 weeks), such as about 4 weeks prior to treatment, an osteoclast number in a tumor sample obtained from the individual has been determined to be lower than a reference osteoclast number.

The compositions utilized in the methods described herein (e.g., PD-L1 axis binding antagonists, anti-CD38 antibodies, and other anti-cancer therapeutic agents) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions described herein can also be administered systemically or locally. The method of administration can vary depending on various factors (e.g., the compound or composition being administered and the severity of the condition, disease, or disorder being treated). In some instances, the PD-L1 axis binding antagonist is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Therapeutic agents, including, e.g., PD-L1 axis binding antagonists, anti-CD38 antibodies, and other anti-cancer therapeutic agents described herein (or any additional therapeutic agent) (e.g., an antibody, binding polypeptide, and/or small molecule) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The therapeutic agent need not be, but is optionally formulated with and/or administered concurrently with one or more agents currently used to prevent or treat the disorder in question. The effective amount of such other agents depends on the amount of the therapeutic agent present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the treatment of a cancer (e.g., a hematologic cancer (e.g., a myeloma (e.g., a multiple myeloma (MM), e.g., a relapsed or refractory MM)), the appropriate dosage of a therapeutic agent (e.g., a PD-L1 axis binding antagonist, a CD38 antagonist, or any other anti-cancer therapeutic agent) described herein (when used alone or in combination with one or more other additional therapeutic agents) will depend on the type of cancer to be treated, the severity and course of the cancer, whether the therapeutic agent is administered for preventive or therapeutic purposes, previous therapy, the patient's clinical history, and the discretion of the attending physician. The therapeutic agent is suitably administered to the patient at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every week or every three weeks (e.g., such that the patient receives, for example, from about two to about twenty, or e.g., about six doses of the therapeutic agent). An initial higher loading dose followed by one or more lower doses may be administered. However, other dosage regimens may be useful. The progress of this therapy is easily monitored by conventional techniques and assays.

For example, as a general proposition, the therapeutically effective amount of an antibody (e.g., an anti-PD-L1 antagonist antibody or a CD38 antagonist antibody) administered to human will be in the range of about 0.01 to about 50 mg/kg of patient body weight, whether by one or more administrations. In some instances, the antibody used is about 0.01 mg/kg to about 45 mg/kg, about 0.01 mg/kg to about 40 mg/kg, about 0.01 mg/kg to about 35 mg/kg, about 0.01 mg/kg to about 30 mg/kg, about 0.01 mg/kg to about 25 mg/kg, about 0.01 mg/kg to about 20 mg/kg, about 0.01 mg/kg to about 15 mg/kg, about 0.01 mg/kg to about 10 mg/kg, about 0.01 mg/kg to about 5 mg/kg, or about 0.01 mg/kg to about 1 mg/kg administered daily, weekly, every two weeks, every three weeks, or monthly, for example. In some instances, the antibody is administered at 15 mg/kg. However, other dosage regimens may be useful. In one instance, an anti-PD-L1 antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, or about 1800 mg on day 1 of 21-day cycles (every three weeks, q3w). In some instances, the anti-PD-L1 antibody atezolizumab is administered at 1200 mg intravenously every three weeks (q3w). In some instances, anti-PD-L1 antibody atezolizumab is administered at 840 mg intravenously every two weeks (q2w). In some instances, anti-PD-L1 antibody atezolizumab is administered at 1680 mg intravenously every four weeks (q4w). The dose may be administered as a single dose or as multiple doses (e.g., 2 or 3 doses), such as infusions. The dose of the antibody administered in a combination treatment may be reduced as compared to a single treatment. The progress of this therapy is easily monitored by conventional techniques.

In some aspects, the effective amount of the anti-PD-L1 antagonist antibody (e.g., an anti-PD-L1 antagonist antibody as disclosed herein, e.g., atezolizumab) is a fixed dose of between about 30 mg to about 1650 mg (e.g., between about 30 mg to about 1650 mg, e.g., between about 50 mg to about 1600 mg, e.g., between about 100 mg to about 1500 mg, e.g., between about 200 mg to about 1400 mg, e.g., between about 300 mg to about 1300 mg, e.g., between about 400 mg to about 1200 mg, e.g., between about 500 mg to about 1100 mg, e.g., between about 600 mg to about 1000 mg, e.g., between about 700 mg to about 900 mg, e.g., between about 800 mg to about 900 mg, e.g., 840 mg±10 mg, e.g., 840±6 mg, e.g., 840±5 mg, e.g., 840±3 mg, e.g., 840±1 mg, e.g., 840±0.5 mg, e.g., 840 mg) every two weeks. In some aspects, the effective amount of the anti-PD-L1 antagonist antibody (e.g., an anti-PD-L1 antagonist antibody as disclosed herein, e.g., atezolizumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the effective amount of the anti-PD-L1 antagonist antibody (e.g., an anti-PD-L1 antagonist antibody as disclosed herein, e.g., atezolizumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some aspects, the effective amount of the anti-PD-L1 antagonist antibody (e.g., an anti-PD-L1 antagonist antibody as disclosed herein, e.g., atezolizumab) is a fixed dose of about 600 mg every three weeks. In some aspects, effective amount of the anti-PD-L1 antagonist antibody (e.g., an anti-PD-L1 antagonist antibody as disclosed herein, e.g., atezolizumab) is a fixed dose of 600 mg.

In some aspects, the effective amount of the anti-CD38 antibody (e.g., an anti-CD38 antagonist antibody, e.g., daratumumab) is a dose of between about 8 mg/kg to about 24 mg/kg of the subject's body weight (e.g., between about 8 mg/kg to about 22 mg/kg, e.g., between about 10 mg/kg to about 20 mg/kg, e.g., between about 10 mg/kg to about 18 mg/kg, e.g., between about 12 mg/kg to about 16 mg/kg, e.g., about 16±2 mg/kg, about 16±1 mg/kg, about 16±0.5 mg/kg, about 16±0.2 mg/kg, or about 16±0.1 mg/kg, e.g., about 16 mg/kg). In some aspects, the effective amount of anti-CD38 antibody (e.g., an anti-CD38 antagonist antibody, e.g., daratumumab) is a dose of about 16 mg/kg.

In any of the methods and uses of the invention, the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) may be administered in a dosing regimen that includes at least nine dosing cycles (e.g., 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the dosing regimen includes at least 12 dosing cycles. In other aspects, the dosing regimen includes at least 16 dosing cycles. In some aspects, the dosing cycles of the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) continue until there is a loss of clinical benefit (e.g., confirmed disease progression, drug resistance, death, or unacceptable toxicity). In some aspects, the length of each dosing cycle is about 15 to 24 days (e.g., 15 days, 16 days, 17 days, 18 days, 19 days, 20 days, 21 days, 22 days, 23 days, or 24 days). In some aspects, the length of each dosing cycle is about 21 days.

In some aspects, the anti-PD-L1 antagonist antibody (e.g., an anti-PD-L1 antagonist antibody as disclosed herein, e.g., atezolizumab) is administered on about day 1 (e.g., day 1±1 day) of each dosing cycle. For example, the anti-PD-L1 antagonist antibody (e.g., an anti-PD-L1 antagonist antibody as disclosed herein, e.g., atezolizumab) is administered intravenously at a fixed dose of about 840 mg on day 2 and day 16 of cycle 1 and on day 1 and day 15 of every 28-day cycle thereafter (i.e., at a fixed dose of about 840 mg every two weeks). In another aspect, the anti-PD-L1 antagonist antibody (e.g., an anti-PD-L1 antagonist antibody as disclosed herein, e.g., atezolizumab) is administered intravenously at a fixed dose of about 600 mg on day 1 of each 21 day cycle (i.e., at a fixed dose of about 600 mg every three weeks). In another aspect, the anti-PD-L1 antagonist antibody (e.g., an anti-PD-L1 antagonist antibody as disclosed herein, e.g., atezolizumab) is administered intravenously at a fixed dose of about 600 mg on day 2 of each 21 day cycle (i.e., at a fixed dose of about 600 mg every three weeks). Similarly, in some aspects, the anti-CD38 antibody (e.g., an anti-CD38 antagonist antibody, e.g., daratumumab) is administered on or about days 1 (e.g., day 1±1 day), 8 (e.g., day 8±1 day), and 15 (e.g., day 15±1 day) of each of dosing cycles 1-3, on or about day 1 (e.g., day 1±1 day) of each of dosing cycles 4-8, and on or about day 1 (e.g., day 1±1 day) of dosing cycle 9. For example, the anti-CD38 antibody is administered intravenously at a dose of 16 mg/kg on each of days 1, 8, and 15 of dosing cycles 1, 2, and 3; on day 1 of each of dosing cycles 4, 5, 6, 7, 8, and 9. In some aspects, the anti-CD38 antibody (e.g., an anti-CD38 antagonist antibody, e.g., daratumumab) is administered once every four weeks beginning on or about day 1 of cycle nine. For example, the anti-CD38 antibody (e.g., an anti-CD38 antagonist antibody, e.g., daratumumab) is administered intravenously at a dose of 16 mg/kg on day 1 of dosing cycle nine, on day 8 of dosing cycle 10, on day 15 of dosing cycle 11, on day 1 of dosing cycle 13, on day 8 of dosing cycle 14, on day 15 of dosing cycle 15, on day 1 of dosing cycle 17, and once every four weeks thereafter. In some aspects, any of the doses of the anti-CD38 antibody (e.g., an anti-CD38 antagonist antibody, e.g., daratumumab) may be split into two doses and administered to the subject over the course of two consecutive days. In some aspects, the first dose of the anti-CD38 antibody (e.g., an anti-CD38 antagonist antibody, e.g., daratumumab) is administered over days 1 and 2 of cycle 1.

In some aspects, when the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) are scheduled to be administered on the same day, the anti-CD38 antibody may be administered either on that day, or on the next consecutive day. Accordingly, in some aspects, the anti-PD-L1 antagonist antibody (e.g., an anti-PD-L1 antagonist antibody as disclosed herein, e.g., atezolizumab) is administered to the subject on day 1 of the dosing cycle and an anti-CD38 antibody (e.g., anti-CD38 antagonist antibody, e.g., daratumumab) is administered to the subject on day 2 of the dosing cycle. In other aspects, the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) are both administered to the subject on day 1 of the dosing cycle. In aspects in which the anti-PD-L1 antagonist antibody (e.g., an anti-PD-L1 antagonist antibody as disclosed herein, e.g., atezolizumab) and an anti-CD38 antibody (e.g., anti-CD38 antagonist antibody, e.g., daratumumab) are both administered to the subject on the same day, the anti-PD-L1 antagonist antibody (e.g., an anti-PD-L1 antagonist antibody as disclosed herein, e.g., atezolizumab) is administered before the anti-CD38 antibody (e.g., an anti-CD38 antagonist antibody, e.g., daratumumab).

In some aspects, the anti-PD-L1 antagonist antibody (e.g., an anti-PD-L1 antagonist antibody as disclosed herein, e.g., atezolizumab) is administered to the subject before the anti-CD38 antibody (e.g., an anti-CD38 antagonist antibody, e.g., daratumumab). In some aspects, for example, following administration of the anti-PD-L1 antagonist antibody and before administration of the anti-CD38 antibody, the method includes an intervening first observation period. In some aspects, the method further includes a second observation period following administration of the anti-CD38 antibody. In some aspects, the method includes both a first observation period following administration of the anti-PD-L1 antagonist antibody and second observation period following administration of the anti-CD38 antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-PD-L1 antagonist antibody and anti-CD38 antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-PD-L1 antagonist antibody and anti-CD38 antibody during the first and second observation periods, respectively.

In other aspects, an anti-CD38 antibody (e.g., anti-CD38 antagonist antibody, e.g., daratumumab) is administered to the subject before the anti-PD-L1 antagonist antibody (e.g., an anti-PD-L1 antagonist antibody as disclosed herein, e.g., atezolizumab). In some aspects, for example, following administration of the anti-CD38 antibody and before administration of the anti-PD-L1 antagonist antibody, the method includes an intervening first observation period. In some aspects, the method includes a second observation period following administration of the anti-PD-L1 antagonist antibody. In some aspects, the method includes both a first observation period following administration of the anti-CD38 antibody and second observation period following administration of the anti-PD-L1 antagonist antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-CD38 antibody and anti-PD-L1 antagonist antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-CD38 antibody and anti-PD-L1 antagonist antibody during the first and second observation periods, respectively.

In some aspects, the methods and uses further include administering to the subject one or more of a corticosteroid (e.g., methylprednisolone), an antipyretic (e.g., acetaminophen), and an antihistamine (e.g., diphenhydramine) prior to each administration of the anti-CD38 antibody (e.g., an anti-CD38 antagonist antibody, e.g., daratumumab). In some aspects, the methods and uses further include administering to the subject a corticosteroid (e.g., methylprednisolone), an antipyretic (e.g., acetaminophen), and an antihistamine (e.g., diphenhydramine) prior to each administration of the anti-CD38 antibody (e.g., an anti-CD38 antagonist antibody, e.g., daratumumab). For example, 100 mg IV methylprednisolone, 650-1000 mg oral acetaminophen, and/or 25-50 mg oral or IV diphenhydramine is administered to the subject about one to three hours prior to the administration of the anti-CD38 antibody. In other aspects, the methods and uses include administering to the subject a corticosteroid on each of the two days following administration of the anti-CD38 antibody (e.g., an anti-CD38 antagonist antibody, e.g., daratumumab), beginning on the day following administration. For example, 20 mg methylprednisolone is administered to the subject on days 1 and 2 following administration of the anti-CD38 antibody.

In another aspect, the invention provides a method of treating a subject having a relapsed or refractory MM by administering to the subject atezolizumab at a fixed dose of 840 mg and daratumumab at a dose of 16 mg/kg in a dosing regimen comprising at least nine dosing cycles, wherein the length of each dosing cycle is 21 days, and wherein (a) the anti-PD-L1 antagonist antibody is administered once every two weeks and (b) the anti-CD38 antibody is administered once every week during each of dosing cycles 1-2, once every two weeks during each of dosing cycles 3-6, and once every four weeks beginning on dosing cycle 7.

In another aspect, the invention provides an anti-PD-L1 antagonist antibody (e.g., an anti-PD-L1 antagonist antibody disclosed herein, e.g., atezolizumab) and anti-CD38 antibody (e.g., an anti-CD38 antagonist antibody, e.g., daratumumab) for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of an anti-PD-L1 antagonist antibody (e.g., an anti-PD-L1 antagonist antibody described herein, e.g., atezolizumab) and an anti-CD38 antibody (e.g., an anti-CD38 antagonist antibody, e.g., daratumumab) in a dosing regimen comprising at least nine dosing cycles, wherein (a) the anti-PD-L1 antagonist antibody is administered once every three weeks; and (b) the anti-CD38 antibody is administered once every week during each of dosing cycles 1-2, once every two weeks during each of dosing cycles 3-6, and once every four weeks beginning on dosing cycle 7.

In some aspects, the effective amount of the anti-CD38 antibody (e.g., an anti-CD38 antagonist antibody, e.g., daratumumab) is a dose of between about 8 mg/kg to about 24 mg/kg of the subject's body weight (e.g., between about 8 mg/kg to about 22 mg/kg, e.g., between about 10 mg/kg to about 20 mg/kg, e.g., between about 10 mg/kg to about 18 mg/kg, e.g., between about 12 mg/kg to about 16 mg/kg, e.g., about 16±2 mg/kg, about 16±1 mg/kg, about 16±0.5 mg/kg, about 16±0.2 mg/kg, or about 16±0.1 mg/kg, e.g., about 16 mg/kg). In some aspects, the effective amount of anti-CD38 antibody (e.g., an anti-CD38 antagonist antibody, e.g., daratumumab) is a dose of about 16 mg/kg.

In any of the methods and uses of the invention, the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) is to be administered in a dosing regimen that includes at least nine dosing cycles (e.g., 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the dosing regimen includes at least 12 dosing cycles. In other aspects, the dosing regimen includes at least 16 dosing cycles. In some aspects, the dosing cycles of the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) continue until there is a loss of clinical benefit (e.g., confirmed disease progression, drug resistance, death, or unacceptable toxicity). In some aspects, the length of each dosing cycle is about 15 to 28 days (e.g., 15 days, 16 days, 17 days, 18 days, 19 days, 20 days, 21 days, 22 days, 23 days, 24 days, 25 days, 26 days, 27 days, or 28 days). In some aspects, the length of each dosing cycle is about 28 days.

In some aspects, when the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) are scheduled to be administered on the same day, the anti-CD38 antibody is to be administered either on that day, or on the next consecutive day. Accordingly, in some aspects, the anti-PD-L1 antagonist antibody (e.g., an anti-PD-L1 antagonist antibody as disclosed herein, e.g., atezolizumab) is to be administered to the subject on day 1 of the dosing cycle and an anti-CD38 antibody (e.g., anti-CD38 antagonist antibody, e.g., daratumumab) is to be administered to the subject on day 2 of the dosing cycle. In other aspects, the anti-PD-L1 antagonist antibody (e.g., an anti-PD-L1 antagonist antibody as disclosed herein, e.g., atezolizumab) and an anti-CD38 antibody (e.g., anti-CD38 antagonist antibody, e.g., daratumumab) are both to be administered to the subject on day 1 of the dosing cycle. In aspects in which the anti-PD-L1 antagonist antibody (e.g., an anti-PD-L1 antagonist antibody as disclosed herein, e.g., atezolizumab) and an anti-CD38 antibody (e.g., anti-CD38 antagonist antibody, e.g., daratumumab) are both to be administered to the subject on the same day, the anti-PD-L1 antagonist antibody (e.g., an anti-PD-L1 antagonist antibody as disclosed herein, e.g., atezolizumab) is to be administered before an anti-CD38 antibody (e.g., anti-CD38 antagonist antibody, e.g., daratumumab).

In some aspects, the anti-PD-L1 antagonist antibody (e.g., an anti-PD-L1 antagonist antibody as disclosed herein, e.g., atezolizumab) is to be administered to the subject before the anti-CD38 antibody (e.g., an anti-CD38 antagonist antibody, e.g., daratumumab). In some aspects, for example, following administration of the anti-PD-L1 antagonist antibody and before administration of the anti-CD38 antibody, the method includes an intervening first observation period. In some aspects, the method further includes a second observation period following administration of the anti-CD38 antibody. In some aspects, the method includes both a first observation period following administration of the anti-PD-L1 antagonist antibody and second observation period following administration of the anti-CD38 antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-PD-L1 antagonist antibody and anti-CD38 antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-PD-L1 antagonist antibody and anti-CD38 antibody during the first and second observation periods, respectively.

In other aspects, an anti-CD38 antibody (e.g., anti-CD38 antagonist antibody, e.g., daratumumab) is to be administered to the subject before the anti-PD-L1 antagonist antibody (e.g., an anti-PD-L1 antagonist antibody as disclosed herein, e.g., atezolizumab). In some aspects, for example, following administration of the anti-CD38 antibody and before administration of the anti-PD-L1 antagonist antibody, the method includes an intervening first observation period. In some aspects, the method includes a second observation period following administration of the anti-PD-L1 antagonist antibody. In some aspects, the method includes both a first observation period following administration of the anti-CD38 antibody and second observation period following administration of the anti-PD-L1 antagonist antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-CD38 antibody and anti-PD-L1 antagonist antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-CD38 antibody and anti-PD-L1 antagonist antibody during the first and second observation periods, respectively.

In some aspects, the method further includes administering to the subject one or more of a corticosteroid (e.g., methylprednisolone), an antipyretic (e.g., acetaminophen), and an antihistamine (e.g., diphenhydramine) prior to each administration of the anti-CD38 antibody (e.g., an anti-CD38 antagonist antibody, e.g., daratumumab). In some aspects, the methods and uses further include administering to the subject a corticosteroid (e.g., methylprednisolone), an antipyretic (e.g., acetaminophen), and an antihistamine (e.g., diphenhydramine) prior to each administration of the anti-CD38 antibody (e.g., an anti-CD38 antagonist antibody, e.g., daratumumab). For example, 100 mg IV methylprednisolone, 650-1000 mg oral acetaminophen, and/or 25-50 mg oral or IV diphenhydramine is to be administered to the subject about one to three hours prior to the administration of the anti-CD38 antibody. In other aspects, the method includes administering to the subject a corticosteroid on each of the two days following administration of the anti-CD38 antibody (e.g., an anti-CD38 antagonist antibody, e.g., daratumumab), beginning on the day following administration. For example, 20 mg methylprednisolone is to be administered to the subject on days 1 and 2 following administration of the anti-CD38 antibody.

In another aspect, the invention provides uses of an effective amount of an anti-PD-L1 antagonist antibody (e.g., an anti-PD-L1 antagonist antibody disclosed herein, e.g., atezolizumab) in the manufacture or preparation of a medicament for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of the medicament comprising the anti-PD-L1 antagonist antibody in combination with an effective amount of an anti-CD38 antibody (e.g., an anti-CD38 antagonist antibody, e.g., daratumumab) in a dosing regimen comprising at least nine dosing cycles, wherein (a) the medicament comprising the anti-PD-L1 antagonist antibody is administered once every two weeks; and (b) the anti-CD38 antibody is administered once every week during each of dosing cycles 1-2, once every three weeks during each of dosing cycles 3-6, and once every four weeks beginning on dosing cycle 7.

In another aspect, the invention provides uses of an effective amount of an anti-CD38 antibody (e.g., an anti-CD38 antagonist antibody, e.g., daratumumab) in the manufacture or preparation of a medicament for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of the medicament comprising the anti-CD38 antibody in combination with an effective amount of an anti-PD-L1 antagonist antibody (e.g., an anti-PD-L1 antagonist antibody disclosed herein, e.g., atezolizumab) in a dosing regimen comprising at least nine dosing cycles, wherein (a) the anti-PD-L1 antagonist antibody is administered once every two weeks; and (b) the medicament comprising the anti-CD38 antibody is administered once every week during each of dosing cycles 1-2, once every three weeks during each of dosing cycles 3-6, and once every four weeks beginning on dosing cycle 7.

In another aspect, the invention provides uses of an effective amount of an anti-PD-L1 antagonist antibody (e.g., an anti-PD-L1 antagonist antibody disclosed herein, e.g., atezolizumab) and an effective amount of an anti-CD38 antibody (e.g., an anti-CD38 antagonist antibody, e.g., daratumumab) in the manufacture or preparation of a medicament for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of the medicament comprising the anti-PD-L1 antagonist antibody in combination with an effective amount of a medicament comprising the anti-CD38 antibody in a dosing regimen comprising at least nine dosing cycles, wherein (a) the medicament comprising the anti-PD-L1 antagonist antibody is administered once every two weeks; and (b) the medicament comprising the anti-CD38 antibody is administered once every week during each of dosing cycles 1-2, once every three weeks during each of dosing cycles 3-6, and once every four weeks beginning on dosing cycle 7.

Any of the methods described herein may further include administering an additional therapeutic agent to the individual. In some aspects, the additional therapeutic agent is selected from the group consisting of an immunotherapy agent, a cytotoxic agent, a growth inhibitory agent, a radiation therapy agent, an anti-angiogenic agent, and combinations thereof. In some instances, the second therapeutic agent is an agonist directed against an activating co-stimulatory molecule. In some instances, the second therapeutic agent is an antagonist directed against an inhibitory co-stimulatory molecule.

CD8⁺ T Cell Density as a Predictive Biomarker for Therapeutic Methods

The invention is based, at least in part, on the discovery that the density of CD8⁺ T cells present in a tumor sample obtained from an individual with a hematologic cancer (e.g., myeloma, e.g., multiple myeloma (MM), e.g., a relapsed or refractory MM) can be used to identify the individual as one who may benefit from a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab). In particular, an individual having a hematologic cancer (e.g., myeloma, e.g., multiple myeloma (MM), e.g., a relapsed or refractory MM) may be identified as likely to benefit from a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab) based on a CD8⁺ T cell density that is higher than a reference CD8⁺ T cell density.

Accordingly, the invention features a method of treating an individual having a hematologic cancer (e.g., myeloma, e.g., multiple myeloma (MM), e.g., a relapsed or refractory MM) who may benefit from a treatment including a PD-L1 axis binding antagonist (e.g., an anti-PD-L1 antibody, e.g., atezolizumab) and an anti-CD38 antibody (e.g., an anti-CD38 antagonist antibody (e.g., daratumumab)), the method including determining a CD8⁺ T cell density in a tumor sample obtained from the individual, wherein a CD8⁺ T cell density that is higher than a reference CD8⁺ T cell density identifies the individual as one who is more likely to benefit from the treatment.

In some embodiments, the CD8⁺ T cell density in the tumor sample from the individual is higher (e.g., by at least about 50 to about 600 objects/mm² area (e.g., about 50, 51, 52, 53, 54, 55, 60, 65, 70, 75, 80, 90, 100, 120, 140, 160, 200, 250, 300, 400, 500, 600 objects/mm² area) than the reference CD8⁺ T cell density and the individual is administered a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab).

In some instances, the method includes treating an individual having a hematologic cancer, the method including: (a) determining a CD8⁺ T cell density in a tumor sample obtained from the individual, wherein the CD8⁺ T cell density in the tumor sample has been determined to be higher than a reference CD8⁺ T cell density; and (b) administering an effective amount of a PD-L1 axis binding antagonist and an anti-CD38 antibody to the individual based on the CD8⁺ T cell density in the tumor sample determined in step (a).

In some instances, the method includes treating an individual having a hematologic cancer (e.g., myeloma, e.g., multiple myeloma (MM), e.g., a relapsed or refractory MM), the method including administering to the individual an effective amount of a PD-L1 axis binding antagonist (e.g., an anti-PD-L1 antibody, e.g., atezolizumab) and an anti-CD38 antibody (e.g., an anti-CD38 antagonist antibody, e.g., daratumumab), wherein prior to treatment, such as, between about 3 days to about 20 weeks (e.g., 3 days, 4 days, 5 days, 6 days, 1 week, 2 weeks, 4 weeks, 8 weeks, 12 weeks, 16 weeks, or 20 weeks), such as about 4 weeks prior to treatment, a CD8⁺ T cell density in a tumor sample obtained from the individual has been determined to be higher (e.g., by at least about 50 to about 600 objects/mm² area (e.g., about 50, 51, 52, 53, 54, 55, 60, 65, 70, 75, 80, 90, 100, 120, 140, 160, 200, 250, 300, 400, 500, 600 objects/mm² area) than a reference CD8⁺ T cell density.

The compositions utilized in the methods described herein (e.g., PD-L1 axis binding antagonists, anti-CD38 antibodies, and other anti-cancer therapeutic agents) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions described herein can also be administered systemically or locally. The method of administration can vary depending on various factors (e.g., the compound or composition being administered and the severity of the condition, disease, or disorder being treated). In some instances, the PD-L1 axis binding antagonist is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Therapeutic agents, including, e.g., PD-L1 axis binding antagonists, anti-CD38 antibodies, and other anti-cancer therapeutic agents described herein (or any additional therapeutic agent) (e.g., an antibody, binding polypeptide, and/or small molecule) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The therapeutic agent need not be, but is optionally formulated with and/or administered concurrently with one or more agents currently used to prevent or treat the disorder in question. The effective amount of such other agents depends on the amount of the therapeutic agent present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the treatment of a cancer (e.g., a hematologic cancer (e.g., a myeloma (e.g., a multiple myeloma (MM), e.g., a relapsed or refractory MM)), the appropriate dosage of a therapeutic agent (e.g., a PD-L1 axis binding antagonist, a CD38 antagonist, or any other anti-cancer therapeutic agent) described herein (when used alone or in combination with one or more other additional therapeutic agents) will depend on the type of cancer to be treated, the severity and course of the cancer, whether the therapeutic agent is administered for preventive or therapeutic purposes, previous therapy, the patient's clinical history, and the discretion of the attending physician. The therapeutic agent is suitably administered to the patient at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every week or every three weeks (e.g., such that the patient receives, for example, from about two to about twenty, or e.g., about six doses of the therapeutic agent). An initial higher loading dose followed by one or more lower doses may be administered. However, other dosage regimens may be useful. The progress of this therapy is easily monitored by conventional techniques and assays.

For example, as a general proposition, the therapeutically effective amount of an antibody (e.g., an anti-PD-L1 antagonist antibody or a CD38 antagonist antibody) administered to human will be in the range of about 0.01 to about 50 mg/kg of patient body weight, whether by one or more administrations. In some instances, the antibody used is about 0.01 mg/kg to about 45 mg/kg, about 0.01 mg/kg to about 40 mg/kg, about 0.01 mg/kg to about 35 mg/kg, about 0.01 mg/kg to about 30 mg/kg, about 0.01 mg/kg to about 25 mg/kg, about 0.01 mg/kg to about 20 mg/kg, about 0.01 mg/kg to about 15 mg/kg, about 0.01 mg/kg to about 10 mg/kg, about 0.01 mg/kg to about 5 mg/kg, or about 0.01 mg/kg to about 1 mg/kg administered daily, weekly, every two weeks, every three weeks, or monthly, for example. In some instances, the antibody is administered at 15 mg/kg. However, other dosage regimens may be useful. In one instance, an anti-PD-L1 antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, or about 1800 mg on day 1 of 21-day cycles (every three weeks, q3w). In some instances, the anti-PD-L1 antibody atezolizumab is administered at 1200 mg intravenously every three weeks (q3w). In some instances, anti-PD-L1 antibody atezolizumab is administered at 840 mg intravenously every two weeks (q2w). In some instances, anti-PD-L1 antibody atezolizumab is administered at 1680 mg intravenously every four weeks (q4w). The dose may be administered as a single dose or as multiple doses (e.g., 2 or 3 doses), such as infusions. The dose of the antibody administered in a combination treatment may be reduced as compared to a single treatment. The progress of this therapy is easily monitored by conventional techniques.

In some aspects, the effective amount of the anti-PD-L1 antagonist antibody (e.g., an anti-PD-L1 antagonist antibody as disclosed herein, e.g., atezolizumab) is a fixed dose of between about 30 mg to about 1650 mg (e.g., between about 30 mg to about 1650 mg, e.g., between about 50 mg to about 1600 mg, e.g., between about 100 mg to about 1500 mg, e.g., between about 200 mg to about 1400 mg, e.g., between about 300 mg to about 1300 mg, e.g., between about 400 mg to about 1200 mg, e.g., between about 500 mg to about 1100 mg, e.g., between about 600 mg to about 1000 mg, e.g., between about 700 mg to about 900 mg, e.g., between about 800 mg to about 900 mg, e.g., 840 mg±10 mg, e.g., 840±6 mg, e.g., 840±5 mg, e.g., 840±3 mg, e.g., 840±1 mg, e.g., 840±0.5 mg, e.g., 840 mg) every two weeks. In some aspects, the effective amount of the anti-PD-L1 antagonist antibody (e.g., an anti-PD-L1 antagonist antibody as disclosed herein, e.g., atezolizumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the effective amount of the anti-PD-L1 antagonist antibody (e.g., an anti-PD-L1 antagonist antibody as disclosed herein, e.g., atezolizumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some aspects, the effective amount of the anti-PD-L1 antagonist antibody (e.g., an anti-PD-L1 antagonist antibody as disclosed herein, e.g., atezolizumab) is a fixed dose of about 600 mg every three weeks. In some aspects, effective amount of the anti-PD-L1 antagonist antibody (e.g., an anti-PD-L1 antagonist antibody as disclosed herein, e.g., atezolizumab) is a fixed dose of 600 mg.

In some aspects, the effective amount of the anti-CD38 antibody (e.g., an anti-CD38 antagonist antibody, e.g., daratumumab) is a dose of between about 8 mg/kg to about 24 mg/kg of the subject's body weight (e.g., between about 8 mg/kg to about 22 mg/kg, e.g., between about 10 mg/kg to about 20 mg/kg, e.g., between about 10 mg/kg to about 18 mg/kg, e.g., between about 12 mg/kg to about 16 mg/kg, e.g., about 16±2 mg/kg, about 16±1 mg/kg, about 16±0.5 mg/kg, about 16±0.2 mg/kg, or about 16±0.1 mg/kg, e.g., about 16 mg/kg). In some aspects, the effective amount of anti-CD38 antibody (e.g., an anti-CD38 antagonist antibody, e.g., daratumumab) is a dose of about 16 mg/kg.

In any of the methods and uses of the invention, the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) may be administered in a dosing regimen that includes at least nine dosing cycles (e.g., 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the dosing regimen includes at least 12 dosing cycles. In other aspects, the dosing regimen includes at least 16 dosing cycles. In some aspects, the dosing cycles of the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) continue until there is a loss of clinical benefit (e.g., confirmed disease progression, drug resistance, death, or unacceptable toxicity). In some aspects, the length of each dosing cycle is about 15 to 24 days (e.g., 15 days, 16 days, 17 days, 18 days, 19 days, 20 days, 21 days, 22 days, 23 days, or 24 days). In some aspects, the length of each dosing cycle is about 21 days.

In some aspects, the anti-PD-L1 antagonist antibody (e.g., an anti-PD-L1 antagonist antibody as disclosed herein, e.g., atezolizumab) is administered on about day 1 (e.g., day 1±1 day) of each dosing cycle. For example, the anti-PD-L1 antagonist antibody (e.g., an anti-PD-L1 antagonist antibody as disclosed herein, e.g., atezolizumab) is administered intravenously at a fixed dose of about 840 mg on day 2 and day 16 of cycle 1 and on day 1 and day 15 of every 28-day cycle thereafter (i.e., at a fixed dose of about 840 mg every two weeks). In another aspect, the anti-PD-L1 antagonist antibody (e.g., an anti-PD-L1 antagonist antibody as disclosed herein, e.g., atezolizumab) is administered intravenously at a fixed dose of about 600 mg on day 1 of each 21 day cycle (i.e., at a fixed dose of about 600 mg every three weeks). In another aspect, the anti-PD-L1 antagonist antibody (e.g., an anti-PD-L1 antagonist antibody as disclosed herein, e.g., atezolizumab) is administered intravenously at a fixed dose of about 600 mg on day 2 of each 21 day cycle (i.e., at a fixed dose of about 600 mg every three weeks). Similarly, in some aspects, the anti-CD38 antibody (e.g., an anti-CD38 antagonist antibody, e.g., daratumumab) is administered on or about days 1 (e.g., day 1±1 day), 8 (e.g., day 8±1 day), and 15 (e.g., day 15±1 day) of each of dosing cycles 1-3, on or about day 1 (e.g., day 1±1 day) of each of dosing cycles 4-8, and on or about day 1 (e.g., day 1±1 day) of dosing cycle 9. For example, the anti-CD38 antibody is administered intravenously at a dose of 16 mg/kg on each of days 1, 8, and 15 of dosing cycles 1, 2, and 3; on day 1 of each of dosing cycles 4, 5, 6, 7, 8, and 9. In some aspects, the anti-CD38 antibody (e.g., an anti-CD38 antagonist antibody, e.g., daratumumab) is administered once every four weeks beginning on or about day 1 of cycle nine. For example, the anti-CD38 antibody (e.g., an anti-CD38 antagonist antibody, e.g., daratumumab) is administered intravenously at a dose of 16 mg/kg on day 1 of dosing cycle nine, on day 8 of dosing cycle 10, on day 15 of dosing cycle 11, on day 1 of dosing cycle 13, on day 8 of dosing cycle 14, on day 15 of dosing cycle 15, on day 1 of dosing cycle 17, and once every four weeks thereafter. In some aspects, any of the doses of the anti-CD38 antibody (e.g., an anti-CD38 antagonist antibody, e.g., daratumumab) may be split into two doses and administered to the subject over the course of two consecutive days. In some aspects, the first dose of the anti-CD38 antibody (e.g., an anti-CD38 antagonist antibody, e.g., daratumumab) is administered over days 1 and 2 of cycle 1.

In some aspects, when the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) are scheduled to be administered on the same day, the anti-CD38 antibody may be administered either on that day, or on the next consecutive day. Accordingly, in some aspects, the anti-PD-L1 antagonist antibody (e.g., an anti-PD-L1 antagonist antibody as disclosed herein, e.g., atezolizumab) is administered to the subject on day 1 of the dosing cycle and an anti-CD38 antibody (e.g., anti-CD38 antagonist antibody, e.g., daratumumab) is administered to the subject on day 2 of the dosing cycle. In other aspects, the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) are both administered to the subject on day 1 of the dosing cycle. In aspects in which the anti-PD-L1 antagonist antibody (e.g., an anti-PD-L1 antagonist antibody as disclosed herein, e.g., atezolizumab) and an anti-CD38 antibody (e.g., anti-CD38 antagonist antibody, e.g., daratumumab) are both administered to the subject on the same day, the anti-PD-L1 antagonist antibody (e.g., an anti-PD-L1 antagonist antibody as disclosed herein, e.g., atezolizumab) is administered before the anti-CD38 antibody (e.g., an anti-CD38 antagonist antibody, e.g., daratumumab).

In some aspects, the anti-PD-L1 antagonist antibody (e.g., an anti-PD-L1 antagonist antibody as disclosed herein, e.g., atezolizumab) is administered to the subject before the anti-CD38 antibody (e.g., an anti-CD38 antagonist antibody, e.g., daratumumab). In some aspects, for example, following administration of the anti-PD-L1 antagonist antibody and before administration of the anti-CD38 antibody, the method includes an intervening first observation period. In some aspects, the method further includes a second observation period following administration of the anti-CD38 antibody. In some aspects, the method includes both a first observation period following administration of the anti-PD-L1 antagonist antibody and second observation period following administration of the anti-CD38 antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-PD-L1 antagonist antibody and anti-CD38 antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-PD-L1 antagonist antibody and anti-CD38 antibody during the first and second observation periods, respectively.

In other aspects, an anti-CD38 antibody (e.g., anti-CD38 antagonist antibody, e.g., daratumumab) is administered to the subject before the anti-PD-L1 antagonist antibody (e.g., an anti-PD-L1 antagonist antibody as disclosed herein, e.g., atezolizumab). In some aspects, for example, following administration of the anti-CD38 antibody and before administration of the anti-PD-L1 antagonist antibody, the method includes an intervening first observation period. In some aspects, the method includes a second observation period following administration of the anti-PD-L1 antagonist antibody. In some aspects, the method includes both a first observation period following administration of the anti-CD38 antibody and second observation period following administration of the anti-PD-L1 antagonist antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-CD38 antibody and anti-PD-L1 antagonist antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-CD38 antibody and anti-PD-L1 antagonist antibody during the first and second observation periods, respectively.

In some aspects, the methods and uses further include administering to the subject one or more of a corticosteroid (e.g., methylprednisolone), an antipyretic (e.g., acetaminophen), and an antihistamine (e.g., diphenhydramine) prior to each administration of the anti-CD38 antibody (e.g., an anti-CD38 antagonist antibody, e.g., daratumumab). In some aspects, the methods and uses further include administering to the subject a corticosteroid (e.g., methylprednisolone), an antipyretic (e.g., acetaminophen), and an antihistamine (e.g., diphenhydramine) prior to each administration of the anti-CD38 antibody (e.g., an anti-CD38 antagonist antibody, e.g., daratumumab). For example, 100 mg IV methylprednisolone, 650-1000 mg oral acetaminophen, and/or 25-50 mg oral or IV diphenhydramine is administered to the subject about one to three hours prior to the administration of the anti-CD38 antibody. In other aspects, the methods and uses include administering to the subject a corticosteroid on each of the two days following administration of the anti-CD38 antibody (e.g., an anti-CD38 antagonist antibody, e.g., daratumumab), beginning on the day following administration. For example, 20 mg methylprednisolone is administered to the subject on days 1 and 2 following administration of the anti-CD38 antibody.

In another aspect, the invention provides a method of treating a subject having a relapsed or refractory MM by administering to the subject atezolizumab at a fixed dose of 840 mg and daratumumab at a dose of 16 mg/kg in a dosing regimen comprising at least nine dosing cycles, wherein the length of each dosing cycle is 21 days, and wherein (a) the anti-PD-L1 antagonist antibody is administered once every two weeks and (b) the anti-CD38 antibody is administered once every week during each of dosing cycles 1-2, once every two weeks during each of dosing cycles 3-6, and once every four weeks beginning on dosing cycle 7.

In another aspect, the invention provides an anti-PD-L1 antagonist antibody (e.g., an anti-PD-L1 antagonist antibody disclosed herein, e.g., atezolizumab) and anti-CD38 antibody (e.g., an anti-CD38 antagonist antibody, e.g., daratumumab) for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of an anti-PD-L1 antagonist antibody (e.g., an anti-PD-L1 antagonist antibody described herein, e.g., atezolizumab) and an anti-CD38 antibody (e.g., an anti-CD38 antagonist antibody, e.g., daratumumab) in a dosing regimen comprising at least nine dosing cycles, wherein (a) the anti-PD-L1 antagonist antibody is administered once every three weeks; and (b) the anti-CD38 antibody is administered once every week during each of dosing cycles 1-2, once every two weeks during each of dosing cycles 3-6, and once every four weeks beginning on dosing cycle 7.

In some aspects, the effective amount of the anti-CD38 antibody (e.g., an anti-CD38 antagonist antibody, e.g., daratumumab) is a dose of between about 8 mg/kg to about 24 mg/kg of the subject's body weight (e.g., between about 8 mg/kg to about 22 mg/kg, e.g., between about 10 mg/kg to about 20 mg/kg, e.g., between about 10 mg/kg to about 18 mg/kg, e.g., between about 12 mg/kg to about 16 mg/kg, e.g., about 16±2 mg/kg, about 16±1 mg/kg, about 16±0.5 mg/kg, about 16±0.2 mg/kg, or about 16±0.1 mg/kg, e.g., about 16 mg/kg). In some aspects, the effective amount of anti-CD38 antibody (e.g., an anti-CD38 antagonist antibody, e.g., daratumumab) is a dose of about 16 mg/kg.

In any of the methods and uses of the invention, the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) is to be administered in a dosing regimen that includes at least nine dosing cycles (e.g., 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the dosing regimen includes at least 12 dosing cycles. In other aspects, the dosing regimen includes at least 16 dosing cycles. In some aspects, the dosing cycles of the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) continue until there is a loss of clinical benefit (e.g., confirmed disease progression, drug resistance, death, or unacceptable toxicity). In some aspects, the length of each dosing cycle is about 15 to 28 days (e.g., 15 days, 16 days, 17 days, 18 days, 19 days, 20 days, 21 days, 22 days, 23 days, 24 days, 25 days, 26 days, 27 days, or 28 days). In some aspects, the length of each dosing cycle is about 28 days.

In some aspects, when the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) are scheduled to be administered on the same day, the anti-CD38 antibody is to be administered either on that day, or on the next consecutive day. Accordingly, in some aspects, the anti-PD-L1 antagonist antibody (e.g., an anti-PD-L1 antagonist antibody as disclosed herein, e.g., atezolizumab) is to be administered to the subject on day 1 of the dosing cycle and an anti-CD38 antibody (e.g., anti-CD38 antagonist antibody, e.g., daratumumab) is to be administered to the subject on day 2 of the dosing cycle. In other aspects, the anti-PD-L1 antagonist antibody (e.g., an anti-PD-L1 antagonist antibody as disclosed herein, e.g., atezolizumab) and an anti-CD38 antibody (e.g., anti-CD38 antagonist antibody, e.g., daratumumab) are both to be administered to the subject on day 1 of the dosing cycle. In aspects in which the anti-PD-L1 antagonist antibody (e.g., an anti-PD-L1 antagonist antibody as disclosed herein, e.g., atezolizumab) and an anti-CD38 antibody (e.g., anti-CD38 antagonist antibody, e.g., daratumumab) are both to be administered to the subject on the same day, the anti-PD-L1 antagonist antibody (e.g., an anti-PD-L1 antagonist antibody as disclosed herein, e.g., atezolizumab) is to be administered before an anti-CD38 antibody (e.g., anti-CD38 antagonist antibody, e.g., daratumumab).

In some aspects, the anti-PD-L1 antagonist antibody (e.g., an anti-PD-L1 antagonist antibody as disclosed herein, e.g., atezolizumab) is to be administered to the subject before the anti-CD38 antibody (e.g., an anti-CD38 antagonist antibody, e.g., daratumumab). In some aspects, for example, following administration of the anti-PD-L1 antagonist antibody and before administration of the anti-CD38 antibody, the method includes an intervening first observation period. In some aspects, the method further includes a second observation period following administration of the anti-CD38 antibody. In some aspects, the method includes both a first observation period following administration of the anti-PD-L1 antagonist antibody and second observation period following administration of the anti-CD38 antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-PD-L1 antagonist antibody and anti-CD38 antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-PD-L1 antagonist antibody and anti-CD38 antibody during the first and second observation periods, respectively.

In other aspects, an anti-CD38 antibody (e.g., anti-CD38 antagonist antibody, e.g., daratumumab) is to be administered to the subject before the anti-PD-L1 antagonist antibody (e.g., an anti-PD-L1 antagonist antibody as disclosed herein, e.g., atezolizumab). In some aspects, for example, following administration of the anti-CD38 antibody and before administration of the anti-PD-L1 antagonist antibody, the method includes an intervening first observation period. In some aspects, the method includes a second observation period following administration of the anti-PD-L1 antagonist antibody. In some aspects, the method includes both a first observation period following administration of the anti-CD38 antibody and second observation period following administration of the anti-PD-L1 antagonist antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-CD38 antibody and anti-PD-L1 antagonist antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-CD38 antibody and anti-PD-L1 antagonist antibody during the first and second observation periods, respectively.

In some aspects, the method further includes administering to the subject one or more of a corticosteroid (e.g., methylprednisolone), an antipyretic (e.g., acetaminophen), and an antihistamine (e.g., diphenhydramine) prior to each administration of the anti-CD38 antibody (e.g., an anti-CD38 antagonist antibody, e.g., daratumumab). In some aspects, the methods and uses further include administering to the subject a corticosteroid (e.g., methylprednisolone), an antipyretic (e.g., acetaminophen), and an antihistamine (e.g., diphenhydramine) prior to each administration of the anti-CD38 antibody (e.g., an anti-CD38 antagonist antibody, e.g., daratumumab). For example, 100 mg IV methylprednisolone, 650-1000 mg oral acetaminophen, and/or 25-50 mg oral or IV diphenhydramine is to be administered to the subject about one to three hours prior to the administration of the anti-CD38 antibody. In other aspects, the method includes administering to the subject a corticosteroid on each of the two days following administration of the anti-CD38 antibody (e.g., an anti-CD38 antagonist antibody, e.g., daratumumab), beginning on the day following administration. For example, 20 mg methylprednisolone is to be administered to the subject on days 1 and 2 following administration of the anti-CD38 antibody.

In another aspect, the invention provides uses of an effective amount of an anti-PD-L1 antagonist antibody (e.g., an anti-PD-L1 antagonist antibody disclosed herein, e.g., atezolizumab) in the manufacture or preparation of a medicament for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of the medicament comprising the anti-PD-L1 antagonist antibody in combination with an effective amount of an anti-CD38 antibody (e.g., an anti-CD38 antagonist antibody, e.g., daratumumab) in a dosing regimen comprising at least nine dosing cycles, wherein (a) the medicament comprising the anti-PD-L1 antagonist antibody is administered once every two weeks; and (b) the anti-CD38 antibody is administered once every week during each of dosing cycles 1-2, once every three weeks during each of dosing cycles 3-6, and once every four weeks beginning on dosing cycle 7.

In another aspect, the invention provides uses of an effective amount of an anti-CD38 antibody (e.g., an anti-CD38 antagonist antibody, e.g., daratumumab) in the manufacture or preparation of a medicament for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of the medicament comprising the anti-CD38 antibody in combination with an effective amount of an anti-PD-L1 antagonist antibody (e.g., an anti-PD-L1 antagonist antibody disclosed herein, e.g., atezolizumab) in a dosing regimen comprising at least nine dosing cycles, wherein (a) the anti-PD-L1 antagonist antibody is administered once every two weeks; and (b) the medicament comprising the anti-CD38 antibody is administered once every week during each of dosing cycles 1-2, once every three weeks during each of dosing cycles 3-6, and once every four weeks beginning on dosing cycle 7.

In another aspect, the invention provides uses of an effective amount of an anti-PD-L1 antagonist antibody (e.g., an anti-PD-L1 antagonist antibody disclosed herein, e.g., atezolizumab) and an effective amount of an anti-CD38 antibody (e.g., an anti-CD38 antagonist antibody, e.g., daratumumab) in the manufacture or preparation of a medicament for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of the medicament comprising the anti-PD-L1 antagonist antibody in combination with an effective amount of a medicament comprising the anti-CD38 antibody in a dosing regimen comprising at least nine dosing cycles, wherein (a) the medicament comprising the anti-PD-L1 antagonist antibody is administered once every two weeks; and (b) the medicament comprising the anti-CD38 antibody is administered once every week during each of dosing cycles 1-2, once every three weeks during each of dosing cycles 3-6, and once every four weeks beginning on dosing cycle 7.

Any of the methods described herein may further include administering an additional therapeutic agent to the individual. In some aspects, the additional therapeutic agent is selected from the group consisting of an immunotherapy agent, a cytotoxic agent, a growth inhibitory agent, a radiation therapy agent, an anti-angiogenic agent, and combinations thereof. In some instances, the second therapeutic agent is an agonist directed against an activating co-stimulatory molecule. In some instances, the second therapeutic agent is an antagonist directed against an inhibitory co-stimulatory molecule.

Use of Activated CD8÷ T Cell Number to Monitor Treatment Responsiveness for Therapeutic Methods

The invention is based, at least in part, on the discovery that the number of activated CD8⁺ T cells (CD8⁺HLA-DR⁺Ki-67⁺ T cells) in the bone marrow can be used to monitor responsiveness of an individual having a hematologic cancer (e.g., a myeloma (e.g., a multiple myeloma (MM), e.g., a relapsed or refractory MM)) to a treatment including a PD-1 axis binding antagonist (e.g., an anti-PD-L1 antibody, e.g., atezolizumab) and an anti-CD38 antibody (e.g., an anti-CD38 antagonist antibody, e.g., daratumumab). In particular, an individual having a hematologic cancer (e.g., myeloma, e.g., multiple myeloma (MM), e.g., a relapsed or refractory MM) may be monitored for responsiveness to a treatment including a PD-L1 axis binding antagonist (e.g., an anti-PD-L1 antibody, e.g., atezolizumab) and an anti-CD38 antibody (e.g., an anti-CD38 antagonist antibody, e.g., daratumumab) based on an increase in the number of activated CD8⁺ T cells.

Accordingly, the invention features a method for monitoring responsiveness of an individual having a hematologic cancer to a treatment comprising a PD-L1 axis binding antagonist and an anti-CD38 antibody, the method including (a) determining the number of activated CD8⁺ T cells in the bone marrow using a biological sample (e.g., bone marrow aspirate) from the individual at a time point following administration (e.g., about 1 minute to about 12 months (e.g., 1 minute, 5 minutes, 10 minutes, 20 minutes, 30 minutes, 40 minutes, 50 minutes, 1 hour, 2 hours, 3 hours, 4 hours, 5 hours, 6 hours, 8 hours, 10 hours, 12 hours, 16 hours, 20 hours, 1 day, 2 days, 3 days, 4 days, 5 days, 6 days, 1 week, 2 weeks, 4 weeks, 8 weeks, 12 weeks, 4 months, 5 months, 6 months, 8 months, 10 months, or 12 months)) of the PD-1 axis binding antagonist (e.g., an anti-PD-L1 antibody, e.g., atezolizumab) and the anti-CD38 antibody (e.g., an anti-CD38 antagonist antibody, e.g., daratumumab); and (b) comparing the number of activated CD8⁺ T cells in the biological sample to a reference number of activated CD8⁺ T cells, wherein an increase (e.g., between at least about 1.1- and about 100-fold (e.g., 1.1-, 1.15-, 1.2-, 1.3-, 1.4-, 1.5-, 1.75-, 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 35-, 40-, 45-, 50-, 60-, 70-, 80-, 90-, or 100-fold)) in the number of activated CD8⁺ T cells in the biological sample (e.g., bone marrow aspirate) relative to the reference number of activated CD8⁺ T cells indicates that the individual is responding to the treatment.

In some instances, the method includes administering a further dose of the PD-L1 axis binding antagonist (e.g., an anti-PD-L1 antibody, e.g., atezolizumab) and the anti-CD38 antibody (e.g., an anti-CD38 antagonist antibody, e.g., daratumumab) to the individual based on the increase in the number of activated CD8⁺ T cells in the biological sample determined in step (b).

The compositions utilized in the methods described herein (e.g., PD-L1 axis binding antagonists, anti-CD38 antibodies, and other anti-cancer therapeutic agents) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions described herein can also be administered systemically or locally. The method of administration can vary depending on various factors (e.g., the compound or composition being administered and the severity of the condition, disease, or disorder being treated). In some instances, the PD-L1 axis binding antagonist is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Therapeutic agents, including, e.g., PD-L1 axis binding antagonists, anti-CD38 antibodies, and other anti-cancer therapeutic agents described herein (or any additional therapeutic agent) (e.g., an antibody, binding polypeptide, and/or small molecule) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The therapeutic agent need not be, but is optionally formulated with and/or administered concurrently with one or more agents currently used to prevent or treat the disorder in question. The effective amount of such other agents depends on the amount of the therapeutic agent present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the treatment of a cancer (e.g., a hematologic cancer (e.g., a myeloma (e.g., a multiple myeloma (MM), e.g., a relapsed or refractory MM)), the appropriate dosage of a therapeutic agent (e.g., a PD-L1 axis binding antagonist, a CD38 antagonist, or any other anti-cancer therapeutic agent) described herein (when used alone or in combination with one or more other additional therapeutic agents) will depend on the type of cancer to be treated, the severity and course of the cancer, whether the therapeutic agent is administered for preventive or therapeutic purposes, previous therapy, the patient's clinical history, and the discretion of the attending physician. The therapeutic agent is suitably administered to the patient at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every week or every three weeks (e.g., such that the patient receives, for example, from about two to about twenty, or e.g., about six doses of the therapeutic agent). An initial higher loading dose followed by one or more lower doses may be administered. However, other dosage regimens may be useful. The progress of this therapy is easily monitored by conventional techniques and assays.

For example, as a general proposition, the therapeutically effective amount of an antibody (e.g., an anti-PD-L1 antagonist antibody or a CD38 antagonist antibody) administered to human will be in the range of about 0.01 to about 50 mg/kg of patient body weight, whether by one or more administrations. In some instances, the antibody used is about 0.01 mg/kg to about 45 mg/kg, about 0.01 mg/kg to about 40 mg/kg, about 0.01 mg/kg to about 35 mg/kg, about 0.01 mg/kg to about 30 mg/kg, about 0.01 mg/kg to about 25 mg/kg, about 0.01 mg/kg to about 20 mg/kg, about 0.01 mg/kg to about 15 mg/kg, about 0.01 mg/kg to about 10 mg/kg, about 0.01 mg/kg to about 5 mg/kg, or about 0.01 mg/kg to about 1 mg/kg administered daily, weekly, every two weeks, every three weeks, or monthly, for example. In some instances, the antibody is administered at 15 mg/kg. However, other dosage regimens may be useful. In one instance, an anti-PD-L1 antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, or about 1800 mg on day 1 of 21-day cycles (every three weeks, q3w). In some instances, the anti-PD-L1 antibody atezolizumab is administered at 1200 mg intravenously every three weeks (q3w). In some instances, anti-PD-L1 antibody atezolizumab is administered at 840 mg intravenously every two weeks (q2w). In some instances, anti-PD-L1 antibody atezolizumab is administered at 1680 mg intravenously every four weeks (q4w). The dose may be administered as a single dose or as multiple doses (e.g., 2 or 3 doses), such as infusions. The dose of the antibody administered in a combination treatment may be reduced as compared to a single treatment. The progress of this therapy is easily monitored by conventional techniques.

In some aspects, the effective amount of the anti-PD-L1 antagonist antibody (e.g., an anti-PD-L1 antagonist antibody as disclosed herein, e.g., atezolizumab) is a fixed dose of between about 30 mg to about 1650 mg (e.g., between about 30 mg to about 1650 mg, e.g., between about 50 mg to about 1600 mg, e.g., between about 100 mg to about 1500 mg, e.g., between about 200 mg to about 1400 mg, e.g., between about 300 mg to about 1300 mg, e.g., between about 400 mg to about 1200 mg, e.g., between about 500 mg to about 1100 mg, e.g., between about 600 mg to about 1000 mg, e.g., between about 700 mg to about 900 mg, e.g., between about 800 mg to about 900 mg, e.g., 840 mg±10 mg, e.g., 840±6 mg, e.g., 840±5 mg, e.g., 840±3 mg, e.g., 840±1 mg, e.g., 840±0.5 mg, e.g., 840 mg) every two weeks. In some aspects, the effective amount of the anti-PD-L1 antagonist antibody (e.g., an anti-PD-L1 antagonist antibody as disclosed herein, e.g., atezolizumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the effective amount of the anti-PD-L1 antagonist antibody (e.g., an anti-PD-L1 antagonist antibody as disclosed herein, e.g., atezolizumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some aspects, the effective amount of the anti-PD-L1 antagonist antibody (e.g., an anti-PD-L1 antagonist antibody as disclosed herein, e.g., atezolizumab) is a fixed dose of about 600 mg every three weeks. In some aspects, effective amount of the anti-PD-L1 antagonist antibody (e.g., an anti-PD-L1 antagonist antibody as disclosed herein, e.g., atezolizumab) is a fixed dose of 600 mg.

In some aspects, the effective amount of the anti-CD38 antibody (e.g., an anti-CD38 antagonist antibody, e.g., daratumumab) is a dose of between about 8 mg/kg to about 24 mg/kg of the subject's body weight (e.g., between about 8 mg/kg to about 22 mg/kg, e.g., between about 10 mg/kg to about 20 mg/kg, e.g., between about 10 mg/kg to about 18 mg/kg, e.g., between about 12 mg/kg to about 16 mg/kg, e.g., about 16±2 mg/kg, about 16±1 mg/kg, about 16±0.5 mg/kg, about 16±0.2 mg/kg, or about 16±0.1 mg/kg, e.g., about 16 mg/kg). In some aspects, the effective amount of anti-CD38 antibody (e.g., an anti-CD38 antagonist antibody, e.g., daratumumab) is a dose of about 16 mg/kg.

In any of the methods and uses of the invention, the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) may be administered in a dosing regimen that includes at least nine dosing cycles (e.g., 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the dosing regimen includes at least 12 dosing cycles. In other aspects, the dosing regimen includes at least 16 dosing cycles. In some aspects, the dosing cycles of the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) continue until there is a loss of clinical benefit (e.g., confirmed disease progression, drug resistance, death, or unacceptable toxicity). In some aspects, the length of each dosing cycle is about 15 to 24 days (e.g., 15 days, 16 days, 17 days, 18 days, 19 days, 20 days, 21 days, 22 days, 23 days, or 24 days). In some aspects, the length of each dosing cycle is about 21 days.

In some aspects, the anti-PD-L1 antagonist antibody (e.g., an anti-PD-L1 antagonist antibody as disclosed herein, e.g., atezolizumab) is administered on about day 1 (e.g., day 1±1 day) of each dosing cycle. For example, the anti-PD-L1 antagonist antibody (e.g., an anti-PD-L1 antagonist antibody as disclosed herein, e.g., atezolizumab) is administered intravenously at a fixed dose of about 840 mg on day 2 and day 16 of cycle 1 and on day 1 and day 15 of every 28-day cycle therafter (i.e., at a fixed dose of about 840 mg every two weeks). In another aspect, the anti-PD-L1 antagonist antibody (e.g., an anti-PD-L1 antagonist antibody as disclosed herein, e.g., atezolizumab) is administered intravenously at a fixed dose of about 600 mg on day 1 of each 21 day cycle (i.e., at a fixed dose of about 600 mg every three weeks). In another aspect, the anti-PD-L1 antagonist antibody (e.g., an anti-PD-L1 antagonist antibody as disclosed herein, e.g., atezolizumab) is administered intravenously at a fixed dose of about 600 mg on day 2 of each 21 day cycle (i.e., at a fixed dose of about 600 mg every three weeks). Similarly, in some aspects, the anti-CD38 antibody (e.g., an anti-CD38 antagonist antibody, e.g., daratumumab) is administered on or about days 1 (e.g., day 1±1 day), 8 (e.g., day 8±1 day), and 15 (e.g., day 15±1 day) of each of dosing cycles 1-3, on or about day 1 (e.g., day 1±1 day) of each of dosing cycles 4-8, and on or about day 1 (e.g., day 1±1 day) of dosing cycle 9. For example, the anti-CD38 antibody is administered intravenously at a dose of 16 mg/kg on each of days 1, 8, and 15 of dosing cycles 1, 2, and 3; on day 1 of each of dosing cycles 4, 5, 6, 7, 8, and 9. In some aspects, the anti-CD38 antibody (e.g., an anti-CD38 antagonist antibody, e.g., daratumumab) is administered once every four weeks beginning on or about day 1 of cycle nine. For example, the anti-CD38 antibody (e.g., an anti-CD38 antagonist antibody, e.g., daratumumab) is administered intravenously at a dose of 16 mg/kg on day 1 of dosing cycle nine, on day 8 of dosing cycle 10, on day 15 of dosing cycle 11, on day 1 of dosing cycle 13, on day 8 of dosing cycle 14, on day 15 of dosing cycle 15, on day 1 of dosing cycle 17, and once every four weeks thereafter. In some aspects, any of the doses of the anti-CD38 antibody (e.g., an anti-CD38 antagonist antibody, e.g., daratumumab) may be split into two doses and administered to the subject over the course of two consecutive days. In some aspects, the first dose of the anti-CD38 antibody (e.g., an anti-CD38 antagonist antibody, e.g., daratumumab) is administered over days 1 and 2 of cycle 1.

In some aspects, when the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) are scheduled to be administered on the same day, the anti-CD38 antibody may be administered either on that day, or on the next consecutive day. Accordingly, in some aspects, the anti-PD-L1 antagonist antibody (e.g., an anti-PD-L1 antagonist antibody as disclosed herein, e.g., atezolizumab) is administered to the subject on day 1 of the dosing cycle and an anti-CD38 antibody (e.g., anti-CD38 antagonist antibody, e.g., daratumumab) is administered to the subject on day 2 of the dosing cycle. In other aspects, the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) are both administered to the subject on day 1 of the dosing cycle. In aspects in which the anti-PD-L1 antagonist antibody (e.g., an anti-PD-L1 antagonist antibody as disclosed herein, e.g., atezolizumab) and an anti-CD38 antibody (e.g., anti-CD38 antagonist antibody, e.g., daratumumab) are both administered to the subject on the same day, the anti-PD-L1 antagonist antibody (e.g., an anti-PD-L1 antagonist antibody as disclosed herein, e.g., atezolizumab) is administered before the anti-CD38 antibody (e.g., an anti-CD38 antagonist antibody, e.g., daratumumab).

In some aspects, the anti-PD-L1 antagonist antibody (e.g., an anti-PD-L1 antagonist antibody as disclosed herein, e.g., atezolizumab) is administered to the subject before the anti-CD38 antibody (e.g., an anti-CD38 antagonist antibody, e.g., daratumumab). In some aspects, for example, following administration of the anti-PD-L1 antagonist antibody and before administration of the anti-CD38 antibody, the method includes an intervening first observation period. In some aspects, the method further includes a second observation period following administration of the anti-CD38 antibody. In some aspects, the method includes both a first observation period following administration of the anti-PD-L1 antagonist antibody and second observation period following administration of the anti-CD38 antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-PD-L1 antagonist antibody and anti-CD38 antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-PD-L1 antagonist antibody and anti-CD38 antibody during the first and second observation periods, respectively.

In other aspects, an anti-CD38 antibody (e.g., anti-CD38 antagonist antibody, e.g., daratumumab) is administered to the subject before the anti-PD-L1 antagonist antibody (e.g., an anti-PD-L1 antagonist antibody as disclosed herein, e.g., atezolizumab). In some aspects, for example, following administration of the anti-CD38 antibody and before administration of the anti-PD-L1 antagonist antibody, the method includes an intervening first observation period. In some aspects, the method includes a second observation period following administration of the anti-PD-L1 antagonist antibody. In some aspects, the method includes both a first observation period following administration of the anti-CD38 antibody and second observation period following administration of the anti-PD-L1 antagonist antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-CD38 antibody and anti-PD-L1 antagonist antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-CD38 antibody and anti-PD-L1 antagonist antibody during the first and second observation periods, respectively.

In some aspects, the methods and uses further include administering to the subject one or more of a corticosteroid (e.g., methylprednisolone), an antipyretic (e.g., acetaminophen), and an antihistamine (e.g., diphenhydramine) prior to each administration of the anti-CD38 antibody (e.g., an anti-CD38 antagonist antibody, e.g., daratumumab). In some aspects, the methods and uses further include administering to the subject a corticosteroid (e.g., methylprednisolone), an antipyretic (e.g., acetaminophen), and an antihistamine (e.g., diphenhydramine) prior to each administration of the anti-CD38 antibody (e.g., an anti-CD38 antagonist antibody, e.g., daratumumab). For example, 100 mg IV methylprednisolone, 650-1000 mg oral acetaminophen, and/or 25-50 mg oral or IV diphenhydramine is administered to the subject about one to three hours prior to the administration of the anti-CD38 antibody. In other aspects, the methods and uses include administering to the subject a corticosteroid on each of the two days following administration of the anti-CD38 antibody (e.g., an anti-CD38 antagonist antibody, e.g., daratumumab), beginning on the day following administration. For example, 20 mg methylprednisolone is administered to the subject on days 1 and 2 following administration of the anti-CD38 antibody.

In another aspect, the invention provides a method of treating a subject having a relapsed or refractory MM by administering to the subject atezolizumab at a fixed dose of 840 mg and daratumumab at a dose of 16 mg/kg in a dosing regimen comprising at least nine dosing cycles, wherein the length of each dosing cycle is 21 days, and wherein (a) the anti-PD-L1 antagonist antibody is administered once every two weeks and (b) the anti-CD38 antibody is administered once every week during each of dosing cycles 1-2, once every two weeks during each of dosing cycles 3-6, and once every four weeks beginning on dosing cycle 7.

In another aspect, the invention provides an anti-PD-L1 antagonist antibody (e.g., an anti-PD-L1 antagonist antibody disclosed herein, e.g., atezolizumab) and anti-CD38 antibody (e.g., an anti-CD38 antagonist antibody, e.g., daratumumab) for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of an anti-PD-L1 antagonist antibody (e.g., an anti-PD-L1 antagonist antibody described herein, e.g., atezolizumab) and an anti-CD38 antibody (e.g., an anti-CD38 antagonist antibody, e.g., daratumumab) in a dosing regimen comprising at least nine dosing cycles, wherein (a) the anti-PD-L1 antagonist antibody is administered once every three weeks; and (b) the anti-CD38 antibody is administered once every week during each of dosing cycles 1-2, once every two weeks during each of dosing cycles 3-6, and once every four weeks beginning on dosing cycle 7.

In some aspects, the effective amount of the anti-CD38 antibody (e.g., an anti-CD38 antagonist antibody, e.g., daratumumab) is a dose of between about 8 mg/kg to about 24 mg/kg of the subject's body weight (e.g., between about 8 mg/kg to about 22 mg/kg, e.g., between about 10 mg/kg to about 20 mg/kg, e.g., between about 10 mg/kg to about 18 mg/kg, e.g., between about 12 mg/kg to about 16 mg/kg, e.g., about 16±2 mg/kg, about 16±1 mg/kg, about 16±0.5 mg/kg, about 16±0.2 mg/kg, or about 16±0.1 mg/kg, e.g., about 16 mg/kg). In some aspects, the effective amount of anti-CD38 antibody (e.g., an anti-CD38 antagonist antibody, e.g., daratumumab) is a dose of about 16 mg/kg.

In any of the methods and uses of the invention, the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) is to be administered in a dosing regimen that includes at least nine dosing cycles (e.g., 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the dosing regimen includes at least 12 dosing cycles. In other aspects, the dosing regimen includes at least 16 dosing cycles. In some aspects, the dosing cycles of the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) continue until there is a loss of clinical benefit (e.g., confirmed disease progression, drug resistance, death, or unacceptable toxicity). In some aspects, the length of each dosing cycle is about 15 to 28 days (e.g., 15 days, 16 days, 17 days, 18 days, 19 days, 20 days, 21 days, 22 days, 23 days, 24 days, 25 days, 26 days, 27 days, or 28 days). In some aspects, the length of each dosing cycle is about 28 days.

In some aspects, when the anti-PD-L1 antagonist antibody (e.g., an anti-PD-L1 antagonist antibody as disclosed herein, e.g., atezolizumab) and the anti-CD38 antibody (e.g., an anti-CD38 antagonist antibody, e.g., daratumumab) are scheduled to be administered on the same day, the anti-CD38 antibody is to be administered either on that day, or on the next consecutive day. Accordingly, in some aspects, the anti-PD-L1 antagonist antibody (e.g., an anti-PD-L1 antagonist antibody as disclosed herein, e.g., atezolizumab) is to be administered to the subject on day 1 of the dosing cycle and an anti-CD38 antibody (e.g., anti-CD38 antagonist antibody, e.g., daratumumab) is to be administered to the subject on day 2 of the dosing cycle. In other aspects, the anti-PD-L1 antagonist antibody (e.g., an anti-PD-L1 antagonist antibody as disclosed herein, e.g., atezolizumab) and an anti-CD38 antibody (e.g., anti-CD38 antagonist antibody, e.g., daratumumab) are both to be administered to the subject on day 1 of the dosing cycle. In aspects in which the anti-PD-L1 antagonist antibody (e.g., an anti-PD-L1 antagonist antibody as disclosed herein, e.g., atezolizumab) and an anti-CD38 antibody (e.g., anti-CD38 antagonist antibody, e.g., daratumumab) are both to be administered to the subject on the same day, the anti-PD-L1 antagonist antibody (e.g., an anti-PD-L1 antagonist antibody as disclosed herein, e.g., atezolizumab) is to be administered before an anti-CD38 antibody (e.g., anti-CD38 antagonist antibody, e.g., daratumumab).

In some aspects, the anti-PD-L1 antagonist antibody (e.g., an anti-PD-L1 antagonist antibody as disclosed herein, e.g., atezolizumab) is to be administered to the subject before the anti-CD38 antibody (e.g., an anti-CD38 antagonist antibody, e.g., daratumumab). In some aspects, for example, following administration of the anti-PD-L1 antagonist antibody and before administration of the anti-CD38 antibody, the method includes an intervening first observation period. In some aspects, the method further includes a second observation period following administration of the anti-CD38 antibody. In some aspects, the method includes both a first observation period following administration of the anti-PD-L1 antagonist antibody and second observation period following administration of the anti-CD38 antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-PD-L1 antagonist antibody and anti-CD38 antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-PD-L1 antagonist antibody and anti-CD38 antibody during the first and second observation periods, respectively.

In other aspects, an anti-CD38 antibody (e.g., anti-CD38 antagonist antibody, e.g., daratumumab) is to be administered to the subject before the anti-PD-L1 antagonist antibody (e.g., an anti-PD-L1 antagonist antibody as disclosed herein, e.g., atezolizumab). In some aspects, for example, following administration of the anti-CD38 antibody and before administration of the anti-PD-L1 antagonist antibody, the method includes an intervening first observation period. In some aspects, the method includes a second observation period following administration of the anti-PD-L1 antagonist antibody. In some aspects, the method includes both a first observation period following administration of the anti-CD38 antibody and second observation period following administration of the anti-PD-L1 antagonist antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-CD38 antibody and anti-PD-L1 antagonist antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-CD38 antibody and anti-PD-L1 antagonist antibody during the first and second observation periods, respectively.

In some aspects, the method further includes administering to the subject one or more of a corticosteroid (e.g., methylprednisolone), an antipyretic (e.g., acetaminophen), and an antihistamine (e.g., diphenhydramine) prior to each administration of the anti-CD38 antibody (e.g., an anti-CD38 antagonist antibody, e.g., daratumumab). In some aspects, the methods and uses further include administering to the subject a corticosteroid (e.g., methylprednisolone), an antipyretic (e.g., acetaminophen), and an antihistamine (e.g., diphenhydramine) prior to each administration of the anti-CD38 antibody (e.g., an anti-CD38 antagonist antibody, e.g., daratumumab). For example, 100 mg IV methylprednisolone, 650-1000 mg oral acetaminophen, and/or 25-50 mg oral or IV diphenhydramine is to be administered to the subject about one to three hours prior to the administration of the anti-CD38 antibody. In other aspects, the method includes administering to the subject a corticosteroid on each of the two days following administration of the anti-CD38 antibody (e.g., an anti-CD38 antagonist antibody, e.g., daratumumab), beginning on the day following administration. For example, 20 mg methylprednisolone is to be administered to the subject on days 1 and 2 following administration of the anti-CD38 antibody.

In another aspect, the invention provides uses of an effective amount of an anti-PD-L1 antagonist antibody (e.g., an anti-PD-L1 antagonist antibody disclosed herein, e.g., atezolizumab) in the manufacture or preparation of a medicament for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of the medicament comprising the anti-PD-L1 antagonist antibody in combination with an effective amount of an anti-CD38 antibody (e.g., an anti-CD38 antagonist antibody, e.g., daratumumab) in a dosing regimen comprising at least nine dosing cycles, wherein (a) the medicament comprising the anti-PD-L1 antagonist antibody is administered once every two weeks; and (b) the anti-CD38 antibody is administered once every week during each of dosing cycles 1-2, once every three weeks during each of dosing cycles 3-6, and once every four weeks beginning on dosing cycle 7.

In another aspect, the invention provides uses of an effective amount of an anti-CD38 antibody (e.g., an anti-CD38 antagonist antibody, e.g., daratumumab) in the manufacture or preparation of a medicament for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of the medicament comprising the anti-CD38 antibody in combination with an effective amount of an anti-PD-L1 antagonist antibody (e.g., an anti-PD-L1 antagonist antibody disclosed herein, e.g., atezolizumab) in a dosing regimen comprising at least nine dosing cycles, wherein (a) the anti-PD-L1 antagonist antibody is administered once every two weeks; and (b) the medicament comprising the anti-CD38 antibody is administered once every week during each of dosing cycles 1-2, once every three weeks during each of dosing cycles 3-6, and once every four weeks beginning on dosing cycle 7.

In another aspect, the invention provides uses of an effective amount of an anti-PD-L1 antagonist antibody (e.g., an anti-PD-L1 antagonist antibody disclosed herein, e.g., atezolizumab) and an effective amount of an anti-CD38 antibody (e.g., an anti-CD38 antagonist antibody, e.g., daratumumab) in the manufacture or preparation of a medicament for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of the medicament comprising the anti-PD-L1 antagonist antibody in combination with an effective amount of a medicament comprising the anti-CD38 antibody in a dosing regimen comprising at least nine dosing cycles, wherein (a) the medicament comprising the anti-PD-L1 antagonist antibody is administered once every two weeks; and (b) the medicament comprising the anti-CD38 antibody is administered once every week during each of dosing cycles 1-2, once every three weeks during each of dosing cycles 3-6, and once every four weeks beginning on dosing cycle 7.

Any of the methods described herein may further include administering an additional therapeutic agent to the individual. In some aspects, the additional therapeutic agent is selected from the group consisting of an immunotherapy agent, a cytotoxic agent, a growth inhibitory agent, a radiation therapy agent, an anti-angiogenic agent, and combinations thereof. In some instances, the second therapeutic agent is an agonist directed against an activating co-stimulatory molecule. In some instances, the second therapeutic agent is an antagonist directed against an inhibitory co-stimulatory molecule.

Combination of Multiple Biomarkers

The methods and use of biomarkers described herein may be used alone or in combination with each other and/or with methods known in the art.

For example, in some aspects, osteoclast number and CD8⁺ T cell density in one or more tumor samples from an individual may be used as a biomarker for any one of the therapeutic methods disclosed herein. In some aspects, osteoclast number in a tumor sample and activated CD8⁺ T cell number in bone marrow from an individual may be used as a biomarker for any one of the therapeutic methods disclosed herein. In some aspects, CD8⁺ T cell density in a tumor sample and activated CD8⁺ T cell number in bone marrow from an individual may be used as a biomarker for any one of the therapeutic methods disclosed herein. In some aspects, osteoclast number in a tumor sample and activated CD8⁺ T cell number in bone marrow from an individual may be used as a biomarker for any one of the therapeutic methods disclosed herein. In some aspects, osteoclast number and CD8⁺ T cell density in one or more tumor samples from an individual and activated CD8⁺ T cell number in bone marrow from the individual may be used as a biomarker for any one of the therapeutic methods disclosed herein.

Additional biomarkers can be used in combination with any of the biomarkers described herein for any one of the therapeutic methods disclosed herein. For example, in some aspects, the number of macrophages present in a tumor sample, blood, or bone marrow from the individual may be used in combination with any of the biomarkers described herein for any one of the therapeutic methods disclosed herein. In some aspects, the expression of immune checkpoint inhibitors by tumor cells, immune cells (e.g., CD8⁺ T cells, CD4⁺ T cells, osteoclasts, or macrophages), or other cells near tumor cells (e.g., fibroblasts) in a sample (e.g., a tumor sample, a blood sample, a bone marrow sample) from the individual may be used in combination with any of the biomarkers described herein for any one of the therapeutic methods disclosed herein. In some aspects, indicia of angiogenesis (e.g., expression of VEGF) or vascularity (e.g., intercapillary distance and microvessel density) in a sample (e.g., a tumor sample, a blood sample, a bone marrow sample) from the individual may be used in combination with any of the biomarkers described herein for any one of the therapeutic methods disclosed herein.

Response Criteria

In some embodiments, therapy with a PD-L1 axis binding antagonist (e.g., an anti-PD-L1 antibody, e.g., atezolizumab) and an anti-CD38 antibody (e.g., an anti-CD38 antagonist antibody, e.g., daratumumab), preferably results in an objective response, wherein the objective response is a stringent complete response (sCR), a complete response (CR), a very good partial response (VGPR), a partial response (PR), or a minimal response (MR) (Table 1). In some embodiments, therapy with a PD-L1 axis binding antagonist (e.g., an anti-PD-L1 antibody, e.g., atezolizumab) and an anti-CD38 antibody (e.g., an anti-CD38 antagonist antibody, e.g., daratumumab) inhibits and/or delays disease progression (Table 2).

TABLE 1
Response Categories According to IMWG Uniform Response Criteria

| | |
|---|---|
| Response | |
| Subcategory | Response Criteria |

All response categories require two consecutive assessments made any time before starting any new
therapy.

| | |
|---|---|
| sCR | CR as defined below, plus: |
| | Normal FLC ratio and absence of clonal cells in BM by immunohistochemistry |
| | (kappa/lamda ratio ≤ 4:1 or ≥ 1:2 for kappa and lambda patients, respectively after |
| | counting ≥ 100 plasma cells)^{a} |
| CR | No evidence of initial monoclonal protein isotype(s) on immunofixation of the serum and |
| | urine,^{b} disappearance of any soft tissue plasmacytomas, and ≤ 5% plasma cells in BM |
| VGPR | Serum and urine M-protein detectable by immunofixation but not on electrophoresis; or |
| | ≥ 90% reduction in serum M-protein- plus urine M-protein level < 100 mg/24 hr |
| PR | ≥ 50% reduction of serum M-protein and reduction in 24 hr urinary M-protein- by ≥ 90% |
| | or to < 200 mg/24 hr. |
| | If the serum and urine M-protein are unmeasurable, a ≥ 50% decrease in the |
| | difference between involved and uninvolved FLC levels is required in place of the |
| | M-protein criteria. |
| | If serum and urine M-protein are unmeasurable and serum FLC assay is also |
| | unmeasurable, > 50% reduction in plasma cells is required in place of M-protein, |
| | provided baseline BM plasma cell percentage was > 30%. |
| | In addition to the above listed criteria, if present at baseline, a > 50% reduction in |
| | the size (SPD)^{c} of soft tissue plasmacytomas is also required. |
| MR | ≥ 25% but ≤ 49% reductions of serum M protein and reduction in 24-hour urine M- |
| | protein by 50%-89% |
| | In addition to the above criteria, if present at baseline, 25%-49% reduction in the |
| | size (SPD)^{c} of soft tissue plasmacytomas is also required. |
| SD | Not meeting criteria for MR, CR, VGPR, PR, or PD |

TABLE 2
Disease Progression and Relapse According to IMWG Uniform Response Criteria

| Relapse | |
|---|---|
| Subcategory | Relapse Criteria |
| PD ^{d, e} | Any increase of ≥ 25% from lowest response value in any one of the following: |
| | Serum M-protein (absolute increase must be ≥ 0.5 g/dL) |
| | Serum M-protein increase ≥ 1 g/dL, if the lowest M component was ≥ 5g/dL |
| | Urine M-protein (absolute increase must be ≥ 200 mg/24 hr) |
| | In patients without measurable serum and urine M-protein levels: the difference |
| | between involved and uninvolved FLC levels (absolute increase must be > 10 |
| | mg/dL) |
| | In patients without measurable serum and urine M-protein levels and without |
| | measurable disease by FLC: BM plasma cell percentage irrespective of baseline |
| | status (absolute % must be ≥ 10%) ^{b} |
| | Appearance of new lesion(s), ≥ 50% increase from nadir in SPD of > 1 lesion, or |
| | ≥ 50% increase in the longest diameter of a previous lesion > 1 cm in short axis |
| | ≥ 50% increase in circulating plasma cells (minimum 200 cells per microliter) if this |
| | is the only measure of disease |
| Clinical | Requires one or more of the following: |
| relapse | Direct indications of increasing disease and/or end organ dysfunction (CRAB |
| | features) ^{f} related to the underlying clonal plasma cell proliferative disorder. It is not |
| | used in calculation of time to progression or PFS but is listed here as something |
| | that can be reported optionally or for use in clinical practice. |
| | Development of new soft tissue plasmacytomas or bone lesions (osteoporotic |
| | fractures do not constitute progression) |
| | Definite increase in the size of existing plasmacytomas or bone lesions. A definite |
| | increase is defined as a 50% (and ≥ 1 cm) increase as measured serially by the |
| | sum of the products of the cross-diameters of the measurable lesion. |
| | Hypercalcemia > 11 mg/dL (2.65 mmol/L) |
| | Decrease in hemoglobin of ≥ 2 g/dL (1.25 mmol/L) not related to therapy or other |
| | non-myeloma related conditions |
| | Rise in serum creatinine by 2 mg/dL or more (177 μmol/L or more) from the start of |
| | therapy and attributable to myeloma |
| | Hyperviscosity related to serum paraprotein |
| Relapse | Any one or more of the following: |
| from CR (to | Reappearance of serum or urine M-protein- by immunofixation or electrophoresis |
| be used | Development of ≥ 5% plasma cells in the BM |
| only if the | Appearance of any other sign of progression (i.e., new plasmacytoma, lytic bone |
| endpoint | lesion, or hypercalcemia) |
| studied is | |
| PFS) ^{c} | |

BM = bone marrow; CR = complete response; CT = computed tomography; FLC = free light chain; M-protein = monoclonal protein; MR = minimal response; MRI = magnetic resonance imaging; PD = progressive disease; PET = positron emission tomography; PFS = progression-free survival; PR = partial response; sCR = stringent complete response; SD = stable disease; SPD = sum of the products of diameters; VGPR = very good partial response.
^{a} Special attention should be given to the emergence of a different M-protein following treatment, especially in the setting of patients having achieved a conventional CR, often related to oligoclonal reconstitution of the immune system. These bands typically disappear over time and in some studies have been associated with a better outcome. Also, appearance of IgGk in patients receiving monoclonal antibodies should be differentiated from the therapeutic antibody.
^{b} In some cases it is possible that the original M-protein light-chain isotype is still detected on immunofixation but the accompanying heavy-chain component has disappeared; this would not be considered a CR even though the heavy-chain component is not detectable, since it is possible that the clone evolved to one that secreted only light chains. Thus, if a patient has IgA lambda myeloma, then to qualify as CR there should be no IgA detectable on serum or urine immunofixation; if free lambda is detected without IgA, then it must be accompanied by a different heavy-chain isotype (IgG, IgM, etc.). Modified from Durie et al. Leukemia. 20(9):1467-73 (2006). This requires two consecutive assessments to be carried out at any time before the institution of any new therapy (Durie et al. Leukemia. 29:2416-7 (2015)).
^{c} Plasmacytoma measurements should be taken from the CT portion of the PET/CT or MRI scans, or dedicated CT scans where applicable. For patients with only skin involvement, the skin lesions should be measured with a ruler. Measurement of tumor size will be determined by the SPD.
^{d} Positive immunofixation alone in a patient previously classified as achieving a CR will not be considered progression. Criteria for relapse from a CR should be used only when calculating disease-free survival.
^{e} In the case where a value is felt to be a spurious result per investigator discretion (e.g., a possible laboratory error), that value will not be considered when determining the lowest value.
^{f} CRAB features = calcium elevation, renal failure, anemia, lytic bone lesions.

### VI. Exemplary Therapeutic Agents for Use in the Methods and Uses of the Invention

Exemplary PD-L1 axis binding antagonists and anti-CD38 antibodies useful for treating an individual (e.g., a human) having cancer (e.g., a hematologic cancer, e.g., a myeloma (e.g., MM, e.g., a relapsed or refractory MM) in accordance with the methods, uses, and compositions for use are described herein.

A. Exemplary PD-L1 Binding Antagonists

The invention provides anti-PD-L1 antagonist antibodies (e.g., atezolizumab) useful for treating cancer (e.g., a hematologic cancer, e.g., a myeloma (e.g., MM, e.g., a relapsed or refractory MM) in an individual (e.g., a human) who has been determined to be one who may benefit from the treatment and/or be responsive to the treatment with an anti-PD-L1 antagonist antibody.

In certain aspects, the anti-PD-L1 antibody is atezolizumab, YW243.55.S70, MDX-1105, MEDI4736 (durvalumab), or MSB0010718C (avelumab). Antibody YW243.55.S70 is an anti-PD-L1 antibody described in WO 2010/077634. MDX-1105, also known as BMS-936559, is an anti-PD-L1 antibody described in WO2007/005874. MEDI4736 is an anti-PD-L1 monoclonal antibody described in WO2011/066389 and US2013/034559. In some embodiments, the anti-PD-L1 antibody is capable of inhibiting binding between PD-L1 and PD-1 and/or between PD-L1 and B7-1. In some embodiments, the anti-PD-L1 antibody is a monoclonal antibody. In some embodiments, the anti-PD-L1 antibody is an antibody fragment selected from the group consisting of Fab, Fab′-SH, Fv, scFv, and (Fab′)₂ fragments. In some embodiments, the anti-PD-L1 antibody is a humanized antibody. In some embodiments, the anti-PD-L1 antibody is a human antibody.

Examples of anti-PD-L1 antibodies useful for the methods of this invention, and methods for making thereof are described in PCT Patent Application Nos. WO 2010/077634, WO 2007/005874, WO 2011/066389, and in US 2013/034559, which are incorporated herein by reference. The anti-PD-L1 antibodies useful in this invention, including compositions containing such antibodies, may be used as a monotherapy or in combination with one or more additional therapeutic agents, e.g., a platinum-based chemotherapy.

Any suitable anti-PD-L1 antibody may be used in the methods and compositions provided herein. Anti-PD-L1 antibodies described in WO 2010/077634 A1 and U.S. Pat. No. 8,217,149 may be used in the methods and compositions provided herein. In some instances, the anti-PD-L1 antibody comprises a heavy chain variable region sequence of SEQ ID NO: 23 and/or a light chain variable region sequence of SEQ ID NO: 24. In a still further instance, provided is an isolated anti-PD-L1 antibody comprising a heavy chain variable region and/or a light chain variable region sequence, wherein:

(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 23)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSS,

and

(b) the light chain sequence has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 24)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR.

In one instance, the anti-PD-L1 antibody comprises a heavy chain variable region comprising an HVR-H1, HVR-H2 and HVR-H3 sequence, wherein:

(a) the HVR-H1 sequence is
(SEQ ID NO: 27)
GFTFSX₁SWIH;
(b) the HVR-H2 sequence is
(SEQ ID NO: 28)
AWIX₂PYGGSX₃YYADSVKG;
(c) the HVR-H3 sequence is
(SEQ ID NO: 19)
RHWPGGFDY;

further wherein: X₁ is D or G; X₂ is S or L; X₃ is T or S. In one specific aspect, X₁ is D; X₂ is S and X₃ is T. In another aspect, the polypeptide further comprises variable region heavy chain framework sequences juxtaposed between the HVRs according to the formula: (FR-H1)-(HVR-H1)-(FR-H2)-(HVR-H2)-(FR-H3)-(HVR-H3)-(FR-H4). In yet another aspect, the framework sequences are derived from human consensus framework sequences. In a further aspect, the framework sequences are VH subgroup III consensus framework. In a still further aspect, at least one of the framework sequences is the following:

(SEQ ID NO: 29)
FR-H1 is EVQLVESGGGLVQPGGSLRLSCAAS
(SEQ ID NO: 30)
FR-H2 is WVRQAPGKGLEWV
(SEQ ID NO: 31)
FR-H3 is RFTISADTSKNTAYLQMNSLRAEDTAVYYCAR
(SEQ ID NO: 14)
FR-H4 is WGQGTLVTVSS

In a still further aspect, the heavy chain polypeptide is further combined with a variable region light chain comprising an HVR-L1, HVR-L2 and HVR-L3, wherein:

(SEQ ID NO: 32)
(a) the HVR-L1 sequence is RASQX₄X₅X₆TX₇X₈A;
(SEQ ID NO: 33)
(b) the HVR-L2 sequence is SASX₉LX₁₀S,;
(SEQ ID NO: 34)
(c) the HVR-L3 sequence is QQX₁₁X₁₂X₁₃X₁₄PX₁₅T;

wherein: X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, F or I; X₁₄ is H, V, P, T or I; X₁₅ is A, W, R, P or T. In a still further aspect, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H; X₁₅ is A.

In a still further aspect, the light chain further comprises variable region light chain framework sequences juxtaposed between the HVRs according to the formula: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the framework sequences are VL kappa I consensus framework. In a still further aspect, at least one of the framework sequence is the following:

(SEQ ID NO: 35)
FR-L1 is DIQMTQSPSSLSASVGDRVTITC
(SEQ ID NO: 36)
FR-L2 is WYQQKPGKAPKLLIY
(SEQ ID NO: 37)
FR-L3 is GVPSRFSGSGSGTDFTLTISSLQPEDFATYYC
(SEQ ID NO: 38)
FR-L4 is FGQGTKVEIKR

In another instance, provided is an isolated anti-PD-L1 antibody or antigen binding fragment comprising a heavy chain and a light chain variable region sequence, wherein:
(a) the heavy chain comprises an HVR-H1, HVR-H2 and HVR-H3, wherein further:

(SEQ ID NO: 27)
(i) the HVR-H1 sequence is GFTFSX₁SWIH;
(SEQ ID NO: 28)
(ii) the HVR-H2 sequence is AWIX₂PYGGSX₃YYADSVKG
(SEQ ID NO: 19)
(iii) the HVR-H3 sequence is RHWPGGFDY,
and

(b) the light chain comprises an HVR-L1, HVR-L2 and HVR-L3, wherein further:

(SEQ ID NO: 32)
(a) the HVR-L1 sequence is RASQX₄X₅X₆TX₇X₈A;
(SEQ ID NO: 33)
(b) the HVR-L2 sequence is SASX₉LX₁₀S,;
(SEQ ID NO: 34)
(c) the HVR-L3 sequence is QQX₁₁X₁₂X₁₃X₁₄PX₁₅T;

wherein: X₁ is D or G; X₂ is S or L; X₃ is T or S; X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, F or I; X₁₄ is H, V, P, T or I; X₁₅ is A, W, R, P or T. In a specific aspect, X₁ is D; X₂ is S and X₃ is T. In another aspect, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H; X₁₅ is A. In yet another aspect, X₁ is D; X₂ is S and X₃ is T, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H and X₁₅ is A.

In a further aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs: 29, 30, 31, and 14. In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences are set forth as SEQ ID NOs: 35, 36, 37, and 38.

In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect, the minimal effector function results from an “effector-less Fc mutation” or aglycosylation. In still a further instance, the effector-less Fc mutation is an N297A or D265A/N297A substitution in the constant region.

In yet another instance, provided is an anti-PD-L1 antibody comprising a heavy chain and a light chain variable region sequence, wherein:
(a) the heavy chain further comprises an HVR-H1, HVR-H2 and an HVR-H3 sequence having at least 85% sequence identity to GFTFSDSWIH (SEQ ID NO: 17), AWISPYGGSTYYADSVKG (SEQ ID NO: 18) and RHWPGGFDY (SEQ ID NO: 19), respectively, or
(b) the light chain further comprises an HVR-L1, HVR-L2 and an HVR-L3 sequence having at least 85% sequence identity to RASQDVSTAVA (SEQ ID NO: 20), SASFLYS (SEQ ID NO: 21) and QQYLYHPAT (SEQ ID NO: 22), respectively.

In a specific aspect, the sequence identity is 86%, 87%, 88%, 89%, 90%, 91%, 92%, 93%, 94%, 95%, 96%, 97%, 98%, 99% or 100%.

In another aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In yet another aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs: 29, 30, 31, and 14. In a still further aspect, the light chain framework sequences are derived from a Kabat kappa I, II, II, or IV subgroup sequence. In a still further aspect, the light chain framework sequences are V_{L} kappa I consensus framework. In a still further aspect, one or more of the light chain framework sequences are set forth as SEQ ID NOs: 35, 36, 37, and 38.

In a further aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

(SEQ ID NO: 39)
FR-H1 EVQLVESGGGLVQPGGSLRLSCAASGFTFS
(SEQ ID NO: 40)
FR-H2 WVRQAPGKGLEWVA
(SEQ ID NO: 31)
FR-H3 RFTISADTSKNTAYLQMNSLRAEDTAVYYCAR
(SEQ ID NO: 14)
FR-H4 WGQGTLVTVSS

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

(SEQ ID NO: 35)
FR-L1 DIQMTQSPSSLSASVGDRVTITC
(SEQ ID NO: 36)
FR-L2 WYQQKPGKAPKLLIY
(SEQ ID NO: 37)
FR-L3 GVPSRFSGSGSGTDFTLTISSLQPEDFATYYC
(SEQ ID NO: 41)
FR-L4 FGQGTKVEIK

In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect the minimal effector function results from an “effector-less Fc mutation” or aglycosylation. In still a further instance, the effector-less Fc mutation is an N297A or D265A/N297A substitution in the constant region.

In yet another instance, provided is an anti-PD-L1 antibody comprising a heavy chain and a light chain variable region sequence, wherein:
(c) the heavy chain further comprises an HVR-H1, HVR-H2 and an HVR-H3 sequence having at least 85% sequence identity to GFTFSDSWIH (SEQ ID NO: 17), AWISPYGGSTYYADSVKG (SEQ ID NO: 18) and RHWPGGFDY (SEQ ID NO: 19), respectively, and/or
(d) the light chain further comprises an HVR-L1, HVR-L2 and an HVR-L3 sequence having at least 85% sequence identity to RASQDVSTAVA (SEQ ID NO: 20), SASFLYS (SEQ ID NO: 21) and QQYLYHPAT (SEQ ID NO: 22), respectively.
   In a specific aspect, the sequence identity is 86%, 87%, 88%, 89%, 90%, 91%, 92%, 93%, 94%, 95%, 96%, 97%, 98%, 99% or 100%.

In another aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In yet another aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs: 29, 30, 31, and WGQGTLVTVSSASTK (SEQ ID NO: 42).

In a still further aspect, the light chain framework sequences are derived from a Kabat kappa I, II, II or IV subgroup sequence. In a still further aspect, the light chain framework sequences are V_{L} kappa I consensus framework. In a still further aspect, one or more of the light chain framework sequences are set forth as SEQ ID NOs: 35, 36, 37, and 38. In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect, the minimal effector function results from an “effector-less Fc mutation” or aglycosylation. In still a further instance, the effector-less Fc mutation is an N297A or D265A/N297A substitution in the constant region.

In a still further instance, provided is an isolated anti-PD-L1 antibody comprising a heavy chain and a light chain variable region sequence, wherein:

(a) the heavy chain sequence has at least 85% sequence identity to the heavy chain sequence:

(SEQ ID NO: 43)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTK,

or

(b) the light chain sequences has at least 85% sequence identity to the light chain sequence:

(SEQ ID NO: 44)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In some instances, provided is an isolated anti-PD-L1 antibody comprising a heavy chain and a light chain variable region sequence, wherein the light chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence of SEQ ID NO: 44. In some instances, provided is an isolated anti-PD-L1 antibody comprising a heavy chain and a light chain variable region sequence, wherein the heavy chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence of SEQ ID NO: 43. In some instances, provided is an isolated anti-PD-L1 antibody comprising a heavy chain and a light chain variable region sequence, wherein the light chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 44 and the heavy chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 43. In some instances, one, two, three, four, or five amino acid residues at the N-terminal of the heavy and/or light chain may be deleted, substituted or modified.

In a still further instance, provided is an isolated anti-PD-L1 antibody comprising a heavy chain and a light chain sequence, wherein:

(a) the heavy chain sequence has at least 85% sequence identity to the heavy chain sequence:

(SEQ ID NO: 45)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG,

and/or

(b) the light chain sequences has at least 85% sequence identity to the light chain sequence:

(SEQ ID NO: 46)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In some instances, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 46. In some instances, provided is an isolated anti-PD-L1 antibody comprising a heavy chain and a light chain sequence, wherein the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 45. In some instances, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 46 and the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 45. In some instances, provided is an isolated anti-PD-L1 antibody comprising a heavy chain comprising the amino acid sequence of SEQ ID NO: 45 and a light chain sequence comprising the amino acid sequence of SEQ ID NO: 46.

In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In any of the instances herein, the isolated anti-PD-L1 antibody can bind to a human PD-L1, for example a human PD-L1 as shown in UniProtKB/Swiss-Prot Accession No. Q9NZQ7.1, or a variant thereof.

In a still further instance, provided is an isolated nucleic acid encoding any of the antibodies described herein. In some instances, the nucleic acid further comprises a vector suitable for expression of the nucleic acid encoding any of the previously described anti-PD-L1 antibodies. In a still further specific aspect, the vector is in a host cell suitable for expression of the nucleic acid. In a still further specific aspect, the host cell is a eukaryotic cell or a prokaryotic cell. In a still further specific aspect, the eukaryotic cell is a mammalian cell, such as Chinese hamster ovary (CHO) cell.

The antibody or antigen binding fragment thereof, may be made using methods known in the art, for example, by a process comprising culturing a host cell containing nucleic acid encoding any of the previously described anti-PD-L1 antibodies or antigen-binding fragments in a form suitable for expression, under conditions suitable to produce such antibody or fragment, and recovering the antibody or fragment.

In another aspect, an anti-PD-L1 antagonist antibody is provided, wherein the antibody comprises a VH as in any of the aspects provided above, and a VL as in any of the aspects provided above, wherein one or both of the variable domain sequences include post-translational modifications.

Examples of anti-PD-L1 antibodies useful for the methods of this invention and methods for making thereof are described in PCT Pub. No: WO 2017/053748, herein incorporated by reference. The anti-PD-L1 antagonist antibodies (e.g., atezolizumab) useful in this invention, including compositions containing such antibodies, may be used in combination with an anti-CD38 antibody to treat a hematologic cancer (e.g., a myeloma (e.g., MM, e.g., a relapsed or refractory MM).

An anti-PD-L1 antagonist antibody according to any of the above aspects may be a monoclonal antibody, comprising a chimeric, humanized, or human antibody. In one aspect, an anti-PD-L1 antagonist antibody is an antibody fragment, for example, a Fv, Fab, Fab′, scFv, diabody, or F(ab′)₂ fragment. In another aspect, the antibody is a full-length antibody, e.g., an intact IgG antibody (e.g., an intact IgG1 antibody) or other antibody class or isotype as defined herein.

In a further aspect, an anti-PD-L1 antagonist antibody according to any of the above aspects may incorporate any of the features, singly or in combination, as described in Sections 1-6 below.

B. Exemplary PD-1 Binding Antagonists

The invention provides PD-1 binding antagonists useful for treating cancer (e.g., a hematologic cancer, e.g., a myeloma (e.g., MM, e.g., a relapsed or refractory MM) in an individual (e.g., a human) who has been determined to be one who may benefit from the treatment and/or be responsive to the treatment with an PD-L1 axis binding antagonist.

In some embodiments, the PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, the PD-L2 binding antagonist is a molecule that inhibits the binding of PD-L2 to its binding partners. In a specific aspect, a PD-L2 binding partner is PD-1. The antagonist may be an antibody, an antigen-binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody). Any suitable anti-PD-1 antibody may be used in the context of the invention. In some embodiments, the anti-PD-1 antibody is selected from the group consisting of MDX-1106 (nivolumab), MK-3475 (pembrolizumab), MEDI-0680 (AMP-514), PDR001, REGN2810, and BGB-108. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 binding antagonist is AMP-224. MDX-1106, also known as MDX-1106-04, ONO-4538, BMS-936558, or nivolumab, is an anti-PD-1 antibody described in WO2006/121168. MK-3475, also known as lambrolizumab, is an anti-PD-1 antibody described in WO2009/114335. AMP-224, also known as B7-DCIg, is a PD-L2-Fc fusion soluble receptor described in WO2010/027827 and WO2011/066342.

In some instances, the anti-PD-1 antibody is MDX-1106. Alternative names for “MDX-1106” include MDX-1106-04, ONO-4538, BMS-936558, and nivolumab. In some instances, the anti-PD-1 antibody is nivolumab (CAS Registry Number: 946414-94-4). In a still further instance, provided is an isolated anti-PD-1 antibody comprising a heavy chain variable region comprising the heavy chain variable region amino acid sequence from SEQ ID NO: 47 and/or a light chain variable region comprising the light chain variable region amino acid sequence from SEQ ID NO: 48. In a still further instance, provided is an isolated anti-PD-1 antibody comprising a heavy chain and/or a light chain sequence, wherein:

(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 47)
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK,

and

(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 48)
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In a still further embodiment, provided is an isolated nucleic acid encoding any of the antibodies described herein. In some embodiments, the nucleic acid further comprises a vector suitable for expression of the nucleic acid encoding any of the previously described anti-PD-1 antibodies. In a still further specific aspect, the vector is in a host cell suitable for expression of the nucleic acid. In a still further specific aspect, the host cell is a eukaryotic cell or a prokaryotic cell. In a still further specific aspect, the eukaryotic cell is a mammalian cell, such as Chinese hamster ovary (CHO) cell.

The antibody or antigen-binding fragment thereof, may be made using methods known in the art, for example, by a process comprising culturing a host cell containing nucleic acid encoding any of the previously described anti-PD-1 antibodies in a form suitable for expression, under conditions suitable to produce such antibody or fragment, and recovering the antibody or fragment, or according to any method described below.

In a further aspect, an anti-PD-1 antibody according to any of the above aspects may incorporate any of the features, singly or in combination, as described in Sections 1-6 below.

C. Exemplary Anti-CD38 Antibodies

The invention provides anti-CD38 antibodies (e.g., an anti-CD38 antagonist antibody, e.g., daratumumab) useful for treating cancer (e.g., a hematologic cancer, e.g., a myeloma (e.g., MM, e.g., a relapsed or refractory MM) in an individual (e.g., a human) who has been determined to be one who may benefit from the treatment and/or be responsive to the treatment with an anti-CD38 antibody.

In certain aspects, the anti-CD38 antibodies includes at least one, two, three, four, five, or six HVRs selected from: (a) an HVR-H1 comprising the amino acid sequence of SFAMS (SEQ ID NO: 1); (b) an HVR-H2 comprising the amino acid sequence of AISGSGGGTYYADSVKG (SEQ ID NO: 2); (c) an HVR-H3 comprising the amino acid sequence of DKILWFGEPVFDY (SEQ ID NO: 3); (d) an HVR-L1 comprising the amino acid sequence of RASQSVSSYLA (SEQ ID NO: 4), (e) an HVR-L2 comprising the amino acid sequence of DASNRAT (SEQ ID NO: 5); and/or (f) an HVR-L3 comprising the amino acid sequence of QQRSNWPPTF (SEQ ID NO: 6), or a combination of one or more of the above HVRs and one or more variants thereof having at least about 90% sequence identity (e.g., 90%, 91%, 92%, 93%, 94%, 95%, 96%, 97%, 98%, or 99% identity) to any one of SEQ ID NOs: 1-6.

In some aspects, any of the above anti-CD38 antibodies includes (a) an HVR-H1 comprising the amino acid sequence of SFAMS (SEQ ID NO: 1); (b) an HVR-H2 comprising the amino acid sequence of AISGSGGGTYYADSVKG (SEQ ID NO: 2); (c) an HVR-H3 comprising the amino acid sequence of DKILWFGEPVFDY (SEQ ID NO: 3); (d) an HVR-L1 comprising the amino acid sequence of RASQSVSSYLA (SEQ ID NO: 4); (e) an HVR-L2 comprising the amino acid sequence of DASNRAT (SEQ ID NO: 5); and (f) an HVR-L3 comprising the amino acid sequence of QQRSNWPPTF (SEQ ID NO: 6).

In some aspects, the anti-CD38 antibody further comprises at least one, two, three, or four of the following light chain variable region framework regions (FRs): an FR-L1 comprising the amino acid sequence of EIVLTQSPATLSLSPGERATLSC (SEQ ID NO: 7); an FR-L2 comprising the amino acid sequence of WYQQKPGQAPRLLIY (SEQ ID NO: 8); an FR-L3 comprising the amino acid sequence of GIPARFSGSGSGTDFTLTISSLEPEDFAVYYC (SEQ ID NO: 9); and/or an FR-L4 comprising the amino acid sequence of GQGTKVEIK (SEQ ID NO: 10), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 7-10. In some aspects, for example, the antibody further comprises an FR-L1 comprising the amino acid sequence of EIVLTQSPATLSLSPGERATLSC (SEQ ID NO: 7); an FR-L2 comprising the amino acid sequence of WYQQKPGQAPRLLIY (SEQ ID NO: 8); an FR-L3 comprising the amino acid sequence of GIPARFSGSGSGTDFTLTISSLEPEDFAVYYC (SEQ ID NO: 9); and an FR-L4 comprising the amino acid sequence of GQGTKVEIK (SEQ ID NO: 10).

In some aspects, the anti-CD38 antibody further comprises at least one, two, three, or four of the following heavy chain variable region FRs: an FR-H1 comprising the amino acid sequence of EVQLLESGGGLVQPGGSLRLSCAVSGFTFN (SEQ ID NO: 11); an FR-H2 comprising the amino acid sequence of WVRQAPGKGLEWVS (SEQ ID NO: 12); an FR-H3 comprising the amino acid sequence of RFTISRDNSKNTLYLQMNSLRAEDTAVYFCAK (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 11-14. In some aspects, the anti-CD38 antibody includes an FR-H1 comprising the amino acid sequence of EVQLLESGGGLVQPGGSLRLSCAVSGFTFN (SEQ ID NO: 11); an FR-H2 comprising the amino acid sequence of WVRQAPGKGLEWVS (SEQ ID NO: 12); an FR-H3 comprising the amino acid sequence of RFTISRDNSKNTLYLQMNSLRAEDTAVYFCAK (SEQ ID NO: 13); and an FR-H4 comprising the amino acid sequence of WGQGTLVTVSS (SEQ ID NO: 14).

In some aspects, the anti-CD38 antibody has a VH domain comprising an amino acid sequence having at least at least 90% sequence identity (e.g., at least 91%, 92%, 93%, 94%, 95%, 96%, 97%, 98%, or 99% sequence identity) to, or the sequence of

(SEQ ID NO: 15)
EVQLLESGGGLVQPGGSLRLSCAVSGFTFNSFAMSWVRQAPGKGLEWVSA
ISGSGGGTYYADSVKGRFTISRDNSKNTLYLQMNSLRAEDTAVYFCAKDK
ILWFGEPVFDYWGQGTLVTVSS

and/or a VL domain comprising an amino acid sequence having at least 90% sequence identity (e.g., at least 91%, 92%, 93%, 94%, 95%, 96%, 97%, 98%, or 99% sequence identity) to, or the sequence of

(SEQ ID NO: 16)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPTFGQ
GTKVEIK

In another aspect, an anti-CD38 antibody is provided, wherein the antibody comprises a VH as in any of the aspects provided above, and a VL as in any of the aspects provided above, wherein one or both of the variable domain sequences include post-translational modifications.

In some aspects, an anti-CD38 antibody may bind to CD38 on the surface of a MM cell and mediate cell lysis through the activation of complement-dependent cytotoxicity, ADCC, antibody-dependent cellular phagocytosis (ADCP), and apoptosis mediated by Fc cross-linking, leading to the depletion of malignant cells and reduction of the overall cancer burden. In some aspects, an anti-CD38 antibody may also modulate CD38 enzyme activity through inhibition of ribosyl cyclase enzyme activity and stimulation of the cyclic adenosine diphosphate ribose (cADPR) hydrolase activity of CD38. In certain aspects, an anti-CD38 antibody that binds to CD38 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain aspects, the anti-CD38 antibody may bind to both human CD38 and chimpanzee CD38.

In some aspects, the methods or uses described herein may include using or administering an isolated anti-CD38 antibody that competes for binding to CD38 with any of the anti-CD38 antibodies described above. For example, the method may include administering an isolated anti-CD38 antibody that competes for binding to CD38 with an anti-CD38 antibody having the following six HVRs: (a) an HVR-H1 comprising the amino acid sequence of SFAMS (SEQ ID NO: 1); (b) an HVR-H2 comprising the amino acid sequence of AISGSGGGTYYADSVKG (SEQ ID NO: 2); (c) an HVR-H3 comprising the amino acid sequence of DKILWFGEPVFDY (SEQ ID NO: 3); (d) an HVR-L1 comprising the amino acid sequence of RASQSVSSYLA (SEQ ID NO: 4), (e) an HVR-L2 comprising the amino acid sequence of DASNRAT (SEQ ID NO: 5); and (f) an HVR-L3 comprising the amino acid sequence of QQRSNWPPTF (SEQ ID NO: 6). The methods described herein may also include administering an isolated anti-CD38 antibody that binds to the same epitope as an anti-CD38 antibody described above.

In certain aspects, the anti-CD38 antibody is daratumumab (DARZALEX®). In other aspects, the anti-CD38 antibody is MOR202 or isatuximab (SAR-650984). Examples of anti-CD38 antibodies useful for the methods of this invention and methods for making thereof are described in U.S. Pat. Nos. 7,829,673; 8,263,746; and 8,153,765; and U.S. Pub. No: 20160067205 A1.

An anti-CD38 antibody according to any of the above aspects may be a monoclonal antibody, comprising a chimeric, humanized, or human antibody. In one aspect, an anti-CD38 antibody is an antibody fragment, for example, a Fv, Fab, Fab′, scFv, diabody, or F(ab′)₂ fragment. In another aspect, the antibody is a full-length antibody, e.g., an intact IgG antibody (e.g., an intact IgG1 antibody) or other antibody class or isotype as defined herein.

In a further aspect, an anti-CD38 antibody according to any of the above aspects may incorporate any of the features, singly or in combination, as described in Sections 1-6 below.

1. Antibody Affinity

In certain aspects, an anti-PD-L1 antagonist antibody, anti-PD-1 antibody, and/or anti-CD38 antibody provided herein has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹M to 10⁻¹³ M).

In one aspect, K_{D} is measured by a radiolabeled antigen binding assay (RIA). In one aspect, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another aspect, K_{D} is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ˜10 response units (RU). In one aspect, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, for example, Chen et al., J. Mol. Biol. 293:865-881 (1999). If the on-rate exceeds 10⁶M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain aspects, an anti-PD-L1 antagonist antibody, anti-PD-1 antibody, and/or anti-CD38 antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain aspects, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

3. Chimeric and Humanized Antibodies

In certain aspects, an anti-PD-L1 antagonist antibody, anti-PD-1 antibody, and/or anti-CD38 antibody provided herein is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain aspects, an anti-PD-L1 antagonist antibody, anti-PD-1 antibody, and/or anti-CD38 antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

An anti-PD-L1 antagonist antibody, anti-PD-1 antibody, and/or anti-CD38 antibody may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naïve repertoire can be cloned (e.g., from human) to provide a single source of antibodies to a wide range of non-self and also self antigens without any immunization as described by Griffiths et al., EMBO J, 12: 725-734 (1993). Finally, naïve libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Anti-PD-L1 antagonist antibodies and/or anti-CD38 antibodies or antibody fragments isolated from human antibody libraries are considered human antibodies or human antibody fragments herein.

6. Antibody Variants

In certain aspects, amino acid sequence variants of the anti-PD-L1 antagonist antibodies, anti-PD-1 antibodies, and/or anti-CD38 antibodies are contemplated. As described in detail herein, anti-PD-L1 antagonist antibodies and/or anti-CD38 antibodies may be optimized based on desired structural and functional properties. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

I. Substitution, Insertion, and Deletion Variants

In certain aspects, anti-PD-L1 antagonist antibody, anti-PD-1 antibody, and/or anti-CD38 antibody variants having one or more amino acid substitutions are provided. Sites of interest for substitutional mutagenesis include the HVRs and FRs. Conservative substitutions are shown in Table 3 under the heading of “preferred substitutions.” More substantial changes are provided in Table 3 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 3
Exemplary and Preferred Amino Acid Substitutions

| Original | Exemplary | Preferred |
|---|---|---|
| Residue | Substitutions | Substitutions |
| Ala (A) | Val; Leu; He | Val |
| Arg (R) | Lys; Gin; Asn | Lys |
| Asn (N) | Gin; His; Asp, Lys; Arg | Gin |
| Asp (D) | Glu; Asn | Glu |
| Cys (C) | Ser; Ala | Ser |
| Gin (Q) | Asn; Glu | Asn |
| Glu (E) | Asp; Gin | Asp |
| Giy (G) | Ala | Ala |
| His (H) | Asn; Gin; Lys; Arg | Arg |
| Ile (I) | Leu; Val; Met; Ala; | Leu |
| | Phe; Norleucine | |
| Leu (L) | Norleucine; Ile; | Ile |
| | Val; Met; Ala; Phe | |
| Lys (K) | Arg; Gin; Asn | Arg |
| Met (M) | Leu; Phe; He | Leu |
| Phe (F) | Trp; Leu; Val; lie; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | lie; Leu; Met; Phe; | Leu |
| | Ala; Norleucine | |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain aspects, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain aspects of the variant VH and VL sequences provided above, each HVR either is unaltered, or includes no more than one, two, or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

II. Glycosylation Variants

In certain aspects, anti-PD-L1 antagonist antibodies, anti-PD-1 antibodies, and/or anti-CD38 antibodies can be altered to increase or decrease the extent to which the antibody is glycosylated. Addition or deletion of glycosylation sites to anti-PD-L1 antagonist antibody and/or anti-CD38 antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody are made in order to create antibody variants with certain improved properties.

In one aspect, anti-PD-L1 antagonist antibody and/or anti-CD38 antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In view of the above, in some aspects, the methods of the invention involve administering to the subject in the context of a fractionated, dose-escalation dosing regimen an anti-PD-L1 antagonist antibody (e.g., an anti-PD-L1 antagonist antibody disclosed herein (e.g., atezolizumab)) and/or anti-CD38 antibody (e.g., an anti-CD38 antagonist antibody, e.g., daratumumab) variant that comprises an aglycosylation site mutation. In some aspects, the aglycosylation site mutation reduces effector function of the antibody. In some aspects, the aglycosylation site mutation is a substitution mutation. In some aspects, the antibody comprises a substitution mutation in the Fc region that reduces effector function. In some aspects, the substitution mutation is at amino acid residue N297, L234, L235, and/or D265 (EU numbering). In some aspects, the substitution mutation is selected from the group consisting of N297G, N297A, L234A, L235A, D265A, and P329G. In some aspects, the substitution mutation is at amino acid residue N297. In a preferred aspect, the substitution mutation is N297A.

Anti-PD-L1 antagonist antibody and/or anti-CD38 antibody variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

III. Fc Region Variants

In certain aspects, one or more amino acid modifications are introduced into the Fc region of an anti-PD-L1 antagonist antibody (e.g., an anti-PD-L1 antagonist antibody disclosed herein (e.g., atezolizumab)), anti-PD-1 antibody, and/or anti-CD38 antibody (e.g., an anti-CD38 antagonist antibody, e.g., daratumumab), thereby generating an Fc region variant (see e.g., US 2012/0251531). The Fc region variant may comprise a human Fc region sequence (e.g., a human IgG1, IgG2, IgG3 or IgG4 Fc region) comprising an amino acid modification (e.g., a substitution) at one or more amino acid positions.

In certain aspects, the invention contemplates an anti-PD-L1 antagonist antibody or antibody anti-CD38 antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcεRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CYTOTOX96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). Clq binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Intl. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain aspects, the proline at position 329 of a wild-type human Fc region in the antibody is substituted with glycine or arginine or an amino acid residue large enough to destroy the proline sandwich within the Fc/Fc.gamma receptor interface that is formed between the proline 329 of the Fc and tryptophan residues Trp 87 and Trp 110 of FcgRIII (Sondermann et al.: Nature 406, 267-273 (20 Jul. 2000)). In certain aspects, the antibody comprises at least one further amino acid substitution. In one aspect, the further amino acid substitution is S228P, E233P, L234A, L235A, L235E, N297A, N297D, or P331S, and still in another aspect the at least one further amino acid substitution is L234A and L235A of the human IgG1 Fc region or S228P and L235E of the human IgG4 Fc region (see e.g., US 2012/0251531), and still in another aspect the at least one further amino acid substitution is L234A and L235A and P329G of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain aspect, an antibody variant comprises an Fc region with one or more amino acid substitutions which improve ADCC, e.g., substitutions at positions 298, 333, and/or 334 of the Fc region (EU numbering of residues).

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424, or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In some aspects the anti-PD-L1 antagonist antibody (e.g., an anti-PD-L1 antagonist antibody disclosed herein (e.g., atezolizumab)), and/or anti-CD38 antibody (e.g., an anti-CD38 antagonist antibody, e.g., daratumumab) comprises an Fc region comprising an N297G mutation.

IV. Cysteine Engineered Antibody Variants

In certain aspects, it is desirable to create cysteine engineered anti-PD-L1 antagonist antibodies, anti-PD-1 antibodies, and/or anti-CD38 antibodies, e.g., “thioMAbs,” in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain aspects, any one or more of the following residues are substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

V. Antibody Derivatives

In certain aspects, an anti-PD-L1 antagonist antibody (e.g., an anti-PD-L1 antagonist antibody or a variant thereof (e.g., atezolizumab)), anti-PD-1 antibody, and/or anti-CD38 antibody (e.g., daratumumab or a variant thereof) provided herein are further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another aspect, conjugates of an antibody and nonproteinaceous moiety that may be selectively heated by exposure to radiation are provided. In one aspect, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

Recombinant Production Methods

Anti-PD-L1 antagonist antibodies (e.g., an anti-PD-L1 antagonist antibody disclosed herein (e.g., atezolizumab)), anti-PD-1 antibodies, and/or anti-CD38 antibodies (e.g., daratumumab) may be produced using recombinant methods and compositions, for example, as described in U.S. Pat. No. 4,816,567, which is incorporated herein by reference in its entirety.

For recombinant production of an anti-PD-L1 antagonist antibody and/or anti-CD38 antibody, nucleic acid encoding an antibody, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0, and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

Immunoconjugates

The invention also provides immunoconjugates comprising an anti-PD-L1 antagonist antibody (e.g., an anti-PD-L1 antagonist antibody disclosed herein (e.g., atezolizumab)), anti-PD-1 antibody, and/or anti-CD38 antibody (e.g., an anti-CD38 antagonist antibody, e.g., daratumumab) conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some aspects, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another aspect, an immunoconjugate comprises an anti-PD-L1 antagonist antibody as described herein (e.g., atezolizumab) and/or anti-CD38 antibody (e.g., an anti-CD38 antagonist antibody, e.g., daratumumab) conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another aspect, an immunoconjugate comprises an anti-PD-L1 antagonist antibody as described herein (e.g., atezolizumab) and/or an anti-CD38 antibody as described herein (e.g., daratumumab) conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example Tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker, or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### VII. Pharmaceutical Compositions and Formulations

Any of the PD-L1 axis binding antagonist (e.g., an anti-PD-L1 antibody, e.g., atezolizumab) and anti-CD38 antibodies (e.g., an anti-CD38 antagonist antibody, e.g., daratumumab) described herein can be used in pharmaceutical compositions and formulations. Pharmaceutical compositions and formulations of an PD-L1 axis binding antagonist (e.g., an anti-PD-L1 antibody, e.g., atezolizumab) or anti-CD38 antibody (e.g., an anti-CD38 antagonist antibody, e.g., daratumumab) can be prepared by mixing such antibodies having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules. The formulations to be used for in vivo administration are generally sterile. Sterility may be readily accomplished, e.g., by filtration through sterile filtration membranes.

### VIII. Articles of Manufacture and Kits

In another aspect of the invention, an article of manufacture or a kit containing materials useful for the treatment and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing, and/or diagnosing the condition and may have a sterile access port (for example the container may be an intravenous solution bag or a vial having a stopper pierceable by a hypodermic injection needle).

The articles of manufacture and kits may include a PD-L1 axis binding antagonist (e.g., an anti-PD-L1 antibody, e.g., atezolizumab) and an anti-CD38 antibody (e.g., an anti-CD38 antagonist antibody, e.g., daratumumab). The label or package insert indicates that the composition is used for treating the condition of choice (e.g., cancer, e.g., a hematologic cancer, e.g., a myeloma (e.g., MM, e.g., a relapsed or refractory MM). Moreover, the article of manufacture may comprise (a) a first container with a composition contained therein, wherein the composition comprises a PD-L1 axis binding antagonist (e.g., an anti-PD-L1 antibody, e.g., atezolizumab); and (b) a second container with a composition contained therein, wherein the composition comprises an anti-CD38 antibody (e.g., an anti-CD38 antagonist antibody, e.g., daratumumab). The article of manufacture in this aspect further comprises a package insert indicating that the compositions can be used to treat a particular condition. Additionally, the article of manufacture may further comprise a third (or fourth) container comprising a pharmaceutically-acceptable buffer, such as bacteriostatic water for injection (BWFI), phosphate-buffered saline, Ringer's solution, and dextrose solution. It may further include other materials desirable from a commercial and user standpoint, including other buffers, diluents, filters, needles, and syringes.

In one aspect, provided is a kit including an anti-PD-L1 antagonist antibody (e.g., an anti-PD-L1 antagonist antibody disclosed herein (e.g., atezolizumab)), an anti-CD38 antibody (e.g., an anti-CD38 antagonist antibody, e.g., daratumumab), and a package insert comprising instructions to administer to the subject having a hematologic cancer (e.g., a myeloma (e.g., MM, e.g., a relapsed or refractory MM)) the anti-PD-L1 antagonist antibody at a fixed dose of between about 30 mg to about 1200 mg and an anti-CD38 antibody at a dose of between about 8 mg/kg to about 24 mg/kg in a dosing regimen comprising at least nine dosing cycles, wherein (a) the anti-PD-L1 antagonist antibody is administered once every two weeks and (b) the anti-CD38 antibody is administered once every week during each of dosing cycles 1-2, once every two weeks during each of dosing cycles 3-6, and once every four weeks beginning on dosing cycle 7.

In another aspect, provided is a kit including an anti-PD-L1 antagonist antibody (e.g., an anti-PD-L1 antagonist antibody disclosed herein (e.g., atezolizumab)), an anti-CD38 antibody (e.g., an anti-CD38 antagonist antibody, e.g., daratumumab), and a package insert comprising instructions to administer to the subject having a MM (e.g., a relapsed or refractory MM) the anti-PD-L1 antagonist antibody at a fixed dose of 840 mg and an anti-CD38 antibody at a dose of 16 mg/kg in a dosing regimen comprising at least nine dosing cycles, wherein the length of each dosing cycle is 21 days, and wherein (a) the anti-PD-L1 antagonist antibody is administered once every two weeks and (b) the anti-CD38 antibody is administered once every week during each of dosing cycles 1-2, once every two weeks during each of dosing cycles 3-6, and once every four weeks beginning on dosing cycle 7.

In another aspect, provided is a kit including atezolizumab, daratumumab, and a package insert comprising instructions to administer to the subject having a MM (e.g., a relapsed or refractory MM) atezolizumab at a fixed dose of 840 mg and daratumumab at a dose of 16 mg/kg in a dosing regimen comprising at least nine dosing cycles, wherein the length of each dosing cycle is 21 days, and wherein (a) atezolizumab is administered once every two weeks and (b) the daratumumab is administered once every week during each of dosing cycles 1-2, once every two weeks during each of dosing cycles 3-6, and once every four weeks beginning on dosing cycle 7.

In another aspect, the invention features a kit including an anti-PD-L1 antagonist antibody (e.g., an anti-PD-L1 antagonist antibody disclosed herein (e.g., atezolizumab)), an anti-CD38 antibody (e.g., an anti-CD38 antagonist antibody, e.g., daratumumab), and a package insert comprising instructions for using the anti-PD-L1 antagonist antibody and anti-CD38 antibody for treating cancer (e.g., a hematologic cancer, e.g., a myeloma (e.g., MM, e.g., a relapsed or refractory MM)) in a subject according to any of the methods disclosed herein.

In another aspect, provided is a kit including an anti-PD-L1 antagonist antibody (e.g., an anti-PD-L1 antagonist antibody disclosed herein (e.g., atezolizumab)) and a package insert comprising instructions to administer to the subject having a hematologic cancer (e.g., a myeloma (e.g., a multiple myeloma (MM), e.g., a relapsed or refractory MM) the anti-PD-L1 antagonist antibody at a fixed dose of between about 30 mg to about 1200 mg in a dosing regimen comprising one or more dosing cycles, wherein the anti-PD-L1 antagonist antibody is administered once every two weeks.

In another aspect, provided is a kit including an anti-PD-L1 antagonist antibody (e.g., an anti-PD-L1 antagonist antibody disclosed herein (e.g., atezolizumab)) and a package insert comprising instructions to administer to the subject having a MM (e.g., a relapsed or refractory MM) the anti-PD-L1 antagonist antibody at a fixed dose of 840 mg in a dosing regimen comprising at one or more dosing cycles, wherein the length of each dosing cycle is 21 days, and wherein the anti-PD-L1 antagonist antibody is administered once every two weeks.

In another aspect, provided is a kit including atezolizumab and a package insert comprising instructions to administer to the subject having a MM (e.g., a relapsed or refractory MM) atezolizumab at a fixed dose of 840 mg in a dosing regimen comprising one or more dosing cycles, wherein the length of each dosing cycle is 21 days, and wherein atezolizumab is administered once every two weeks. In some aspects, the instructions may further indicate that atezolizumab is to be administered as a monotherapy.

In another aspect, the invention features a kit including an anti-PD-L1 antagonist antibody (e.g., an anti-PD-L1 antagonist antibody disclosed herein (e.g., atezolizumab)) and a package insert comprising instructions for using the anti-PD-L1 antagonist antibody for treating cancer (e.g., a hematologic cancer (e.g., a myeloma (e.g., a multiple myeloma (MM), e.g., a relapsed or refractory MM) in a subject according to any of the methods disclosed herein.

In any of the above aspects, the subject may, for example, be a human. It is specifically contemplated that any of the PD-L1 axis binding antagonists (e.g., an anti-PD-L1 antibody, e.g., atezolizumab) or anti-CD38 antibodies (e.g., an anti-CD38 antagonist antibody, e.g., daratumumab) described herein may be included in the kit.

### EXAMPLES

The following are examples of the methods of the invention. It is understood that various other aspects may be practiced, given the general descriptions provided above.

### Example 1. A Study of the Safety and Pharmacokinetics of Atezolizumab (Anti-PD-L1 Antibody) Alone or in Combination with an Immunomodulatory Drug and/or Daratumumab in Patients with Multiple Myeloma (Relapsed/Refractory and Post-Autologous Stem Cell Transplantation)

Despite advances with the introduction of novel agents such as lenalidomide and proteasome inhibitors added to an autologous stem cell transplantation (ASCT) for a subset of patients who are eligible, many patients fail to achieve an optimal response and typically all patients eventually relapse.

Treatment of refractory patients remains challenging because of disease heterogeneity and the lack of clear understanding of the mechanisms that lead to resistance. With the approval for daratumumab, and other anti-CD38 monoclonal antibodies in development, there will be a growing need for treatment options for patients who fail these therapies. This protocol evaluates the feasibility and tolerability of administering atezolizumab and various atezolizumab combinations in both the relapsed or refractory patient population.

This multicenter, open-label, Phase I study evaluates the safety, efficacy, and pharmacokinetics of atezolizumab alone or in combination with daratumumab and/or various immunomodulatory agents in participants with MM who have relapsed or who have undergone ASCT.

Atezolizumab (also known as MPDL3280A) is a humanized IgG1 monoclonal antibody consisting of two heavy chains (448 amino acids) and two light chains (214 amino acids) and is produced in Chinese hamster ovary cells. Atezolizumab was engineered to eliminate Fc-effector function via a single amino acid substitution (asparagine to alanine) at position 298 on the heavy chain, which results in a non-glycosylated antibody that has minimal binding to Fc receptors and prevents Fc-effector function at expected concentrations in humans. Atezolizumab targets human programmed death-ligand 1 (PD-L1) and inhibits its interaction with its receptors, programmed death-1 (PD-1) and B7.1 (CD80, B7-1). Both of these interactions are reported to provide inhibitory signals to T cells. Without wishing to be bound by one particular theory or mechanism of action, atezolizumab may bind to PD-L1 present on MM cells, thereby enhancing the magnitude and quality of tumor-specific T-cell responses, resulting in improved anti-tumor activity.

The daratumumab, lenalidomide, and dexamethasone regimen was highly active with an ORR of 81%, and 34% of the patients had a sCR or CR. Analysis of correlative studies revealed that daratumumab has immunomodulatory properties because treatment caused robust expansion of peripheral blood and bone marrow T cells and increased T-cell receptor clonality. Without wishing to be bound by one particular theory or mechanism of action, daratumumab binds CD38 present on MM cells, thereby increasing their immunogenicity and enhancing anti-tumor T cell responses.

Objectives and Endpoints

i. Primary Efficacy Objective
The primary efficacy objective for this study is to evaluate the efficacy of atezolizumab administered alone or in combination with lenalidomide; daratumumab; lenalidomide and daratumumab; or pomalidomide and daratumumab based on the following endpoints:
   ORR, as defined as a best overall response of sCR, CR, VGPR, or PR, as defined by IMWG criteria
   To determine the recommended Phase II dose of lenalidomide in combination with atezolizumab, lenalidomide in combination with atezolizumab and daratumumab
   To determine the recommended Phase II dose of pomalidomide in combination with atezolizumab and daratumumab

ii. Secondary Efficacy Objective
The secondary efficacy objectives for this study are to evaluate the efficacy of atezolizumab administered alone or in combination with lenalidomide; daratumumab; lenalidomide and daratumumab; or pomalidomide and daratumumab based on the following endpoints:
   Duration of response, defined as the time from the first observation that a patient achieved a response (sCR, CR, VGPR, or PR), until the date of first recorded progression or death from any cause
   PFS, defined as the time from the start of treatment to the date of the first recorded disease progression (per IMWG criteria) or death from any cause
   ORR at 6, 9, and 12 months, defined as the proportion of patients who have achieved and maintained a sCR, CR, VGPR or PR at 6, 9 and 12 months, respectively, in the study as determined by the investigator with the use of IMWG criteria (Kumar et al. 2016) OS, defined as the time from the start of treatment to death from any cause

iii. Exploratory Biomarker Objective

The exploratory biomarker objective for this study is the identification and profiling of biomarkers associated with disease biology; the mechanism of action of atezolizumab alone and in combination with lenalidomide, daratumumab, lenalidomide/pomalidomide; mechanisms of resistance to atezolizumab alone and in combination with daratumumab and/or lenalidomide/pomalidomide; pharmacodynamics; prognosis; and improvement of diagnostic assays based on the following endpoint:
Relationship between biomarkers in blood and bone marrow (may include somatic mutations) and efficacy, safety, PK, immunogenicity, or other biomarker endpoints

iv. Immunogenicity Objective

The immunogenicity objective for this study is evaluate the immune response to atezolizumab and daratumumab based on the following endpoint:
Incidence of ADAs during the study relative to the prevalence of ADAs at baseline

v. Safety Objectives

The safety objective for this study is to evaluate the safety of atezolizumab administered alone or in combination with lenalidomide; daratumumab; lenalidomide and daratumumab; or pomalidomide and daratumumab based on the following endpoints:
Incidence of adverse events, with severity determined through use of National Cancer Institute Common Terminology Criteria for Adverse Events Version 4.0
Change from baseline in targeted vital signs
Change from baseline in targeted clinical laboratory test results
Change from baseline in physical examination findings

vi. Pharmacokinetic Objective

The Pharmacokinetic objective for this study is to characterize the pharmacokinetics of atezolizumab, lenalidomide, pomalidomide, and daratumumab based on the following endpoint:
Serum concentration of atezolizumab, lenalidomide, pomalidomide, and daratumumab at specified timepoints

Study Design

On the basis of extensive experience with atezolizumab in solid tumors, it is expected that atezolizumab monotherapy should be safe and tolerable in patients with multiple myeloma. However, the effectiveness of atezolizumab alone in multiple myeloma is less clear. Therefore, the approach of this study is to test atezolizumab alone and in combination with various backbone treatments (e.g., IMiDs and/or daratumumab or daratumumab alone) in order to identify promising, safe, and tolerable novel therapies for advanced clinical development.

This is a multicenter, open-label, Phase I study of atezolizumab, alone or in combination, in two populations of patients with MM; those with disease that has relapsed or is refractory and those with measurable disease after receipt of an ASCT. In patients with relapsed or refractory disease and who have received 3 or fewer lines of prior therapy (except for Cohorts D3 and F), the following treatment regimens will be investigated:
Cohort A: atezolizumab alone
Cohort B: atezolizumab and lenalidomide
   Cohort B1: dose escalation
Cohort D: atezolizumab and daratumumab
   Cohort D1: safety run-in
   Cohort D2: expansion
   Cohort D3: expansion (≥2 lines of prior therapy and progression on treatment with an anti-CD38 monoclonal antibody, either alone or in combination)
Cohort E: atezolizumab, daratumumab, and lenalidomide
   Cohort E1: dose escalation
   Cohort E2: expansion

In patients with relapsed or refractory disease who have received 4 or more lines of prior therapy the following treatment regimen will be investigated:
Cohort F: atezolizumab, daratumumab, and pomalidomide
   Cohort F1: dose escalation
   Cohort F2: expansion
   Cohort F3: expansion control arm (daratumumab, pomalidomide, dexamethasone)

Dose and Schedule

Rationale for Atezolizumab Dose and Schedule

The target exposure for atezolizumab was projected on the basis of clinical and nonclinical parameters, including nonclinical tissue distribution data in tumor-bearing mice, target-receptor occupancy in the tumor, and observed atezolizumab interim pharmacokinetics in humans. The target trough concentration (C_{trough}) was projected to be 6 μg/mL on the basis of several assumptions which include that: 1) 95% tumor receptor saturation is needed for efficacy and 2) the tumor-interstitial concentration to plasma ratio is 0.30 based on tissue distribution data in tumor-bearing mice.

In Study PCD4989g, the first-in-human study in patients with advanced solid tumors and hematologic malignancies, 30 patients were treated with atezolizumab at doses that had a range of 0.01-20 mg/kg q3w administered during the dose-escalation stage, and 247 patients were treated with atezolizumab at doses of 10, 15, or 20 mg/kg q3w during the dose-expansion stage. Anti-tumor activity has been observed across doses that had a range of 1-20 mg/kg. There was no evidence of dose-dependent toxicity in Study PCD4989g. The maximum tolerated dose of atezolizumab was not reached, and no dose-limiting toxicities were observed.

ADAs to atezolizumab were associated with changes in pharmacokinetics for some patients in the lower-dose cohorts (0.3, 1, and 3 mg/kg), but patients treated with 10-, 15-, and 20-mg/kg doses maintained the expected C_{trough} despite the detection of ADAs. After review of available PK and ADA data for a range of doses, 15 mg/kg q3w (equivalent to 1200 mg q3w or 840 mg q2w) was identified as an atezolizumab dosing regimen able to maintain C_{trough} at ≥6 μg/mL and further safeguard against interpatient variability and potential ADAs to lead to subtherapeutic levels of atezolizumab.

Simulations do not suggest any clinically meaningful differences in exposure following a fixed-dose compared with a body weight-adjusted dose. Therefore, patients in this study are treated q3w at a fixed dose of 1200 mg or q2w at a fixed dose of 840 mg (both are equivalent to an average body weight-based dose of 15 mg/kg).

Rationale for Lenalidomide/Pomalidomide Dose Escalation

IMiDs have well-known immunomodulatory properties and could be synergistic or additive when combined with atezolizumab and/or daratumumab. There is also a risk for increased immune-mediated adverse events. Therefore, several doses of lenalidomide or pomalidomide in combination with atezolizumab are being explored. The lenalidomide starting dose of 10 mg is equivalent to the dose used in post-ASCT maintenance. Three dose levels of lenalidomide will be initially explored in combination with atezolizumab, with the highest dose equivalent to the standard dose of lenalidomide prescribed to patients with multiple myeloma. In the atezolizumab, daratumumab, and lenalidomide combination, two dose levels of lenalidomide will be explored. Two dose levels of pomalidomide will be explored in combination with atezolizumab and daratumumab, with the highest dose equivalent to the standard dose of pomalidomide prescribed to multiple myeloma patients. Daratumumab has been safely combined with standard doses of lenalidomide (25 mg) and pomalidomide (4 mg).

Rationale for Daratumumab Dose

Daratumumab will be given at the standard dose per local prescribing information.

Inclusion Criteria

General Inclusion Criteria (All Cohorts)

Patients must meet the following criteria for study entry:
Age≥18 years
Given voluntary written informed consent before performance of any study-related procedures not part of normal medical care
Previously diagnosed with MM based on standard criteria
Patients enrolled in Cohorts A, B, C, D1, and E must have received at least one, but not more than three, prior lines of therapy. For the purposes of this study, induction chemotherapy, consolidation with ASCT, maintenance therapy with lenalidomide alone at a dose of no more than 15 mg daily will be considered collectively as one line of therapy. ASCT more than 6 months after completion of induction chemotherapy or undertaken for progression of disease (i.e., salvage therapy) will be considered a separate line of therapy. Post-ASCT with lenalidomide at a dose greater than 15 mg daily or in combination with another agent (e.g., dexamethasone) will be considered a separate line of therapy.
Patients enrolled in Cohort D2 must have received two but not more than three prior lines of therapy that must have included a proteasome inhibitor and an IMiD (alone or in combination) and be refractory to the last line of treatment.
Patients enrolled in Cohort D3 must have received two or more lines of prior therapy, be refractory to both a proteasome inhibitor and an IMiD, and have progressed on treatment (as defined by IMWG criteria) with an anti-CD38 monoclonal antibody (e.g., daratumumab, isatuximab, MOR202) either as a single agent or as a combination. The most recent regimen must have contained an anti-CD38 monoclonal antibody and patients must have achieved at least a minimal response (per IMWG criteria) with anti-CD38-containing therapy.
Patients enrolled in Cohort F must have received four or more lines of prior therapy and be refractory to the last line of treatment.
Relapsed disease, defined as previously treated myeloma that progresses and requires the initiation of salvage therapy, but does not meet criteria for “primary refractory disease” or “relapsed and refractory” disease or
Refractory disease, defined as disease that is non-responsive to salvage therapy or progresses within 60 days following completion of the most recent therapy with achievement of at least a minimal response (MR) or better before disease progression
Willing and able to undergo BM aspiration and biopsy tissue sample collection during screening and while in the study. Pre-treatment evaluable tissue is required for study entry.
Eastern Cooperative Oncology Group (ECOG) performance status score 2
Measurable disease defined as at least one of the following:
Serum M protein≥0.5 g/dL (≥5 g/L)
Urine M protein (≥200 mg/24 hr
Serum free light chains (sFLC) assay: Involved sFLCs≥10 mg/dL (≥100 mg/L) and an abnormal sFLC ratio (<0.26 or >1.65)
Baseline cardiac left ventricular ejection fraction is 40% by either echocardiography or multi-gated angiography scan (MUGA)
Negative serum or urine pregnancy test result for women of childbearing potential
For women of childbearing potential: agreement to remain abstinent (refrain from heterosexual intercourse) or use contraceptive methods that result in a failure rate of <1% per year during the treatment period and for at least 5 months after the last dose of atezolizumab or 90 days after the last dose of daratumumab, or 30 days after the last dose of lenalidomide or pomalidomide, whichever is longer
   A woman is considered to be of childbearing potential if she is postmenarcheal, has not reached a postmenopausal state (≥12 continuous months of amenorrhea with no identified cause other than menopause), and has not undergone surgical sterilization (removal of ovaries and/or uterus).
   Examples of contraceptive methods with a failure rate of <1% per year include bilateral tubal ligation, male sterilization, established, and proper use of hormonal contraceptives that inhibit ovulation, hormone-releasing intrauterine devices, and copper intrauterine devices.
   The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not acceptable methods of contraception.
For men: agreement to remain abstinent (refrain from heterosexual intercourse) or use contraceptive measures and agreement to refrain from donating sperm, as defined below:
   With female partners of childbearing potential or pregnant female partners, men must remain abstinent or use a condom during the treatment period and for at least 90 days after the last dose of lenalidomide or pomalidomide. Men must refrain from donating sperm during this same period.
   The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not acceptable methods of contraception.
No contraindications to atezolizumab.

Cohort A-, B-, D-, E-, and F-Specific Inclusion Criteria: Relapsed or Refractory Patient Population

In addition to meeting the general inclusion criteria for all cohorts, patients in Cohorts A, B, D, E, and F must also meet the following clinical laboratory test result inclusion criteria within the timepoints stipulated in the schedule of study assessments:
ANC≥1000 cells/μL (growth factor cannot be used within the previous 7 days)
AST, ALT and ALP≤2.5×upper limit of normal (ULN), with the following exceptions:
Patients with documented extramedullary liver involvement: AST and ALT≤5×ULN
Patients with documented extramedullary liver involvement or extensive bone involvement: ALP≤5×ULN
Platelet count≥50,000/μL (without platelet transfusion in the previous 7 days); ≥30,000/μL (if myeloma bone marrow involvement is 50%)
Total bilirubin≤2×ULN (patients with known Gilbert's disease who have serum bilirubin≤3×ULN may be enrolled).
Creatinine≤2.0 mL/dL and creatinine clearance (CrCl)≥40 mL/min (calculated or per 24 hour urine collection). For patients who receive lenalidomide: CrCI 60 mL/min, using the Cockcroft-Gault formula.
Serum calcium (corrected for albumin) level at or below the ULN (treatment of hypercalcemia is allowed and patient may enroll if hypercalcemia returns to normal with standard treatment).

Cohort B-, C-, E-, and F-Specific Inclusion Criteria: Relapsed or Refractory Patient Population

In addition to meeting the general inclusion criteria for all cohorts and Cohort A-, B-, E-, and F-specific inclusion criteria, patients in Cohorts B, E, and F must also meet the following entry inclusion criteria:
All patients who are prescribed lenalidomide or pomalidomide must be counseled at a minimum of every 21-28 days about pregnancy precautions and risks of fetal exposure. All patients in Cohorts B1, C, E1, or E2 must agree to be registered in and must comply with all requirements of the Revlimid Risk Evaluation and Mitigation Strategy® (REMS) program. All patients enrolled in Cohorts F1 and F2 must agree to be registered and comply with all requirements of the Pomalyst REMS™ program.
For women of childbearing potential: agreement to remain abstinent or use contraception methods that result in a failure rate of <1% per year during the treatment period and for 5 months after the last dose of atezolizumab or 90 days after the last dose of daratumumab, whichever is longer.
Women of childbearing potential must have a negative serum or urine pregnancy test result. Within 7 days of the pregnancy test, women of childbearing potential enrolled in Cohorts B1, C, E1, E2, F1, or F2 must use two effective methods of contraception for 4 weeks before the start of therapy, during therapy, through the 4 weeks after the last dose of lenalidomide or pomalidomide therapy was administered, and during a dose interruption, unless the patient commits to absolute and continuous abstinence that is confirmed on a monthly basis. If the patient has not established the use of an effective contraception method, the patient must be referred to an appropriately trained health care professional for contraceptive advice so that an effective method of contraception can be initiated.
As a result of the increased risk of venous thromboembolism in patients with MM taking lenalidomide and dexamethasone, and to a lesser extent in patients with myelodysplastic syndromes taking lenalidomide monotherapy, combined oral contraceptive pills are not recommended. If a patient is currently using combined oral contraception the patient should switch to one of the following effective birth control methods:
   Levonorgestrel-releasing intrauterine system
   Medroxyprogesterone acetate depot
   Tubal sterilization
   Sexual intercourse with a vasectomized male partner only; vasectomy must be confirmed by two negative semen analyses
   Ovulation inhibitory progesterone-only pills (i.e., desogestrel)
   The risk of venous thromboembolism continues for 4-6 weeks after discontinuing combined oral contraception.

Cohort C-Specific Inclusion Criteria: Post-ASCT without Progression Patient Population

In addition to meeting the inclusion criteria for all cohorts, patients in Cohort C must also meet the following entry inclusion criteria:
Patients must have recovered sufficiently from their first or second ASCT (preferably between 60 and 90 days, but ≥60 days and not >120 days post-autologous transplant) to initiate atezolizumab maintenance therapy (screening may begin between days 61-120 post-autologous transplant, but must begin no later than Day 121 post-autologous transplant).
Mucositis and gastrointestinal symptoms resolved, off hyperalimentation and IV hydration
Off antibiotics and amphotericin B formulations, voriconazole or other anti-fungal therapy for treatment of proven, probable, or possible infections (defined in accordance with the European Organisation for Research and Treatment of Cancer/Mycoses Study Group 2008 criteria (De Pauw et al. 2008)) for ≥14 days. Patients who completed treatment for an infection but are continuing antibiotics or anti-fungal therapy for prophylaxis are eligible to continue in the study with approval of the Sponsor.
Completed administration of any radiotherapy
Platelet count≥75×10⁹/L (without transfusion in previous 7 days)
ANC≥1.5×10⁹/L without filgrastim administration within 7 days, or peg-filgrastim within 14 days of measurement
AST, ALT, and ALP≤2.5×ULN
Total bilirubin≤2×ULN (patients with known Gilbert disease who have serum bilirubin≤3×ULN may be enrolled).

Creatinine≤2.0 mL/dL and calculated creatinine (CrCl)≥40 mL/min (calculated or per 24-hour urine collection). For patients who receive lenalidomide:
CrCl≥60 mL/min with the use of the Cockcroft-Gault formula or measured per 24-hour urine collection.
Serum calcium (corrected for albumin) level at or below the ULN (treatment of hypercalcemia is allowed and patient may enroll if hypercalcemia returns to normal with standard treatment).

Exclusion Criteria

General Exclusion Criteria (All Cohorts)

Patients who meet any of the following criteria are excluded from study entry:
History of other malignancy within 2 years prior to screening, except those with negligible risk of metastasis or death (e.g., 5-year OS≥90%), such as ductal carcinoma in situ not requiring chemotherapy, appropriately treated carcinoma in situ of the cervix, non-melanoma skin carcinoma, low-grade, localized prostate cancer (Gleason score≤7) not requiring treatment or appropriately treated Stage I uterine cancer
Prior therapy with atezolizumab or other immunotherapeutics, including CD137 agonists, anti-PD-1, anti-CTLA-4, and anti-PD-L1 therapeutic antibodies
Uncontrolled cancer pain. Patients requiring pain medication must be on a stable regimen at study entry. Symptomatic lesions amenable to palliative radiotherapy (e.g., bony lesions or plasmacytoma) should be treated prior to enrollment.
Treatment with any investigational drug within 30 days or 5 half-lives of the investigational drug, whichever is longer
History of severe allergic anaphylactic reactions to chimeric, human or humanized antibodies, or fusion proteins or a known hypersensitivity to biopharmaceuticals produced in CHO cells or any component of the atezolizumab or daratumumab formulations
Prior diagnoses of autoimmune disease including but not limited to uncontrolled autoimmune thyroid disease or Type 1 diabetes, systemic lupus erythematosis, Sjögren's syndrome, glomerulonephritis, multiple sclerosis, rheumatoid arthritis, vasculitis, idiopathic pulmonary fibrosis (IPF, including bronchiolitis obliterans organizing pneumonia), and inflammatory bowel disease, are excluded from study participation. Patients with autoimmune thyroid disease and Type 1 diabetes that is well controlled on a stable medication regimen may be eligible for the study.
Prior systemic, anti-myeloma therapy within 14 days of Cycle 1, Day 1
Primary refractory MM defined as disease that is non-responsive in patients who have never achieved a minimal response or better with any therapy
Prior treatment with chimeric antigen receptor (CAR) T cells or other forms of adoptive cellular therapy, with the exception of autologous stem cell transplantation
POEMS syndrome (polyneuropathy, organomegaly, endocrinopathy, monoclonal protein and skin changes)
Plasma cell leukemia (>2.0×10⁹/L circulating plasma cells by standard differential)
Any Grade>1 (according to the NCI CTCAE v.4.0) adverse reaction unresolved from previous treatments or not readily managed and controlled with supportive care. The presence of alopecia or peripheral neuropathy≤Grade 2 without pain is allowed.
Previous allogeneic stem cell transplant or solid organ transplant
Immunosuppressive therapy (not limited to but including azathioprine, mycophenolate mofetil, cyclosporine, tacrolimus, methotrexate, and anti-tumor necrosis factor (TNF) agents) within 6 weeks of Cycle 1, Day 1
Daily requirement for corticosteroids (>10 mg prednisone daily or equivalent) (except for inhalation corticosteroids) within 2 weeks prior to Cycle 1, Day 1
Positive HIV test at screening
Active hepatitis B virus (HBV) (chronic or acute, defined as having a positive hepatitis B surface antigen (HBsAg) test at screening)
Patients with a past or resolved HBV infection, defined as having a negative HBsAg test and a positive total hepatitis B core antibody (HBcAb) test at screening, are eligible for the study if active HBV infection is ruled out on the basis of HBV DNA viral load per local guidelines.
Active hepatitis C virus (HCV) Patients who have a positive HCV antibody test are eligible for the study if a polymerase chain reaction assay is negative for HCV RNA.
Clinically significant cardiovascular disease (e.g., uncontrolled or any New York Heart Association Class 3 or 4, congestive heart failure, uncontrolled angina, history of myocardial infarction or stroke within 6 months of study entry, uncontrolled hypertension or clinically significant arrhythmias not controlled by medication)
LVEF<40%
Uncontrolled, clinically significant pulmonary disease (e.g., chronic obstructive pulmonary disease, pulmonary hypertension, IPF) that in the opinion of the investigator would put the patient at significant risk for pulmonary complications during the study
History of pneumonitis
Uncontrolled intercurrent illness including, but not limited to uncontrolled infection, disseminated intravascular coagulation, or psychiatric illness/social situations that would limit compliance with study requirements
Pregnant or breastfeeding females
Receipt of a live, attenuated vaccine (e.g., FluMist®) within 4 weeks prior to Cycle 1, Day 1 or anticipation that such a live, attenuated vaccine will be required during the study
Influenza vaccination should be given during influenza season only (approximately October through May in the Northern Hemisphere and approximately April through September in the Southern Hemisphere). Patients must agree not to receive live, attenuated influenza vaccine (e.g., FluMist®) within 28 days prior to initiation of study treatment, during treatment, or within 5 months following the last dose of atezolizumab (for patients randomized to atezolizumab).
Serious infection requiring oral or IV antibiotics within 14 days prior to enrollment (discussion with the Medical Monitor is encouraged in cases where further clarification may be required) Patients on prophylactic antibiotics, antifungals and antivirals in the absence of documented infection are eligible
Any serious medical condition or abnormality in clinical laboratory tests that, in the investigator's or Medical Monitor's judgment, precludes the patient's safe participation in and completion of the study, or which could affect compliance with the protocol or interpretation of results

Cohort B-, C-, E-, and F-Specific Exclusion Criteria

In addition to the exclusion criteria for all cohorts, patients in Cohorts B, C, E, and F who meet any of the following criteria are excluded from the study:
History of erythema multiforme or severe hypersensitivity to prior IMiD's such as thalidomide, lenalidomide, or pomalidomide
Inability to tolerate thromboprophylaxis

Cohort C-Specific Exclusion Criteria

In addition to the exclusion criteria for all cohorts, patients in Cohort C who meet any of the following criteria are excluded from the study:
Evidence of progressive MM compared to pretransplant evaluation as demonstrated by any of the following:
Hypercalcemia defined as serum calcium>25 mmol/L (>1 mg/dL) higher than the ULN or >2.875 mmol/L (>11.5 mg/dL)
New renal failure as defined by CRCL<40 mL/min (measured or calculated from validated formula such as Cockroft-Gault) or worsening renal failure compared to baseline of 20% decrease in CRCL that cannot be explained by concomitant medical condition
Anemia as defined by hemoglobin (Hgb)≤10 gm/dL or ≥2 gm/dL below the lower limit of normal that cannot be explained by concomitant medical condition
New lytic bone lesions or biopsy proven plasmacytomas

Cohort D-, E-, and F-Specific Exclusion Criteria

In addition to the exclusion criteria for all cohorts, patients in Cohorts D1, D2, D3, E, and F who meet any of the following criteria are excluded from the study:
Prior treatment with any anti-CD38 therapy, including daratumumab (except Cohort D3)
Patient has known chronic obstructive pulmonary disease (COPD) with a forced expiratory volume in 1 second (FEV1)<50% of predicted normal. Note that FEV1 testing is required for patients suspected of having COPD and patients must be excluded if FEV1<50% of predicted normal.
Patient has known moderate or severe persistent asthma within the past 2 years, or currently has uncontrolled asthma of any classification. Note that patients who currently have controlled intermittent asthma or controlled mild persistent asthma are allowed in the study.
Screening ECG showing a baseline-corrected QT interval (QTc)>470 msec

Efficacy Analyses

The following analyses to determine the activity of anti-PD-L1 antagonist antibody as a single agent or in combination with the anti-CD38 antibody will be based on the definitions of objective response according to the International Myeloma Working Group Uniform Response (IMWG) criteria (adapted from Durie et al. 2015 and Kumar et al. 2016) for MM or the Lugano Response Criteria for Malignant Lymphoma for DLBCL/FL. Response assessments will be assessed on the basis of physical examinations. CT scans, fluorodeoxyglucose (FDG) positron emission tomography (PET) scans, PET/CT scans, and/or MRI scans, and bone marrow examinations, according to the IMWG response criteria for MM and the Lugano classification for DLBCL/FL.

Response assessment data, progression-free survival, duration of overall response, and OS will be tabulated and listed for all treated patients by disease cohort and treatment. Time to event data will be summarized with Kaplan-Meier curves.

Overall response is defined as a sCR, CR, VGPR, or PR as determined by investigator assessment with the use of the 2016 update of IMWG response criteria. Patients with missing or non-evaluable response assessments will be included into the denominator (total number of patients assessed) in calculations of response rates. The OR rate will be calculated and its 95% CI will be estimated using the Clopper-Pearson method.

Among patients with a response, DOR will be defined as the time from the date of the first observation that a patient achieved the initial sCR, CR, VGPR, or PR to the date of the first recorded disease progression or death. If a patient does not experience death or disease progression before the end of the study, DOR will be censored at the day of the last tumor assessment. If no tumor assessments were performed after the date of the first recorded occurrence of a sCR, CR, PR or VGPR, DOR will be censored at the date of the first occurrence of the OR. PFS is defined as the time from the first day of study treatment to the date of the first recorded disease progression or death, whichever occurs first. If a patient has not experienced PD or death at the time of the data cutoff for analysis, PFS will be censored at the day of the last tumor assessment. Patients with no post-baseline tumor assessments will be censored at the date of first study treatment for non-randomized patients plus 1 day.

For specific cohorts, predictive and/or posterior probabilities will be used to support interpretation and decision-making: posterior probabilities at the final analysis and predictive probabilities at interim analyses.

Interim analyses may be incorporated to guide potential early stopping of enrollment in the expansion cohorts. Predictive and/or posterior probabilities will be used to compare the efficacy endpoints as defined by IMWG criteria in the cohorts D2, E2 and D3 with those of historical controls. The design is based on Lee and Liu (2008), with the modification that the uncertainty in the historical control data is fully taken into account by utilizing a distribution on the control response rate. Interim analysis decision rules will be based on the predictive probability that this trial will have a positive outcome if carried out to completion. The latest information on efficacy of existing therapies in comparable R/R MM patients available at the time of analysis will be used as historical controls for comparison. The possible data sources to be used as historical controls may be the publications, RWD sources, and other reliable information on efficacy from other studies in similar R/R MM patient groups that will be available by the time of the interim analysis. If at any time, interim analysis suggests that predictive probability for positive outcome at the end of the study in a certain cohort is too low, the Sponsor will review the data and decide whether to recommend stopping enrollment in that cohort.

For Cohort D3, interim analysis may be performed after the first 20 and 40 patients for futility, as well as to make a decision on cohort expansion of up to 100 patients. Bayesian posterior probability analysis may also be performed at the 100-patient stage to compare efficacy endpoints, in this cohort, with efficacy data in comparable patient populations from the latest available historical data at the moment of analysis. Currently available data indicates that the historical ORR based on IMWG criteria is 31.1% in R/R patients with 1-12 previous lines or treatment on daratumumab monotherapy (n=148) (Usmani et al. 2016), 33.3% in daratumumab refractory patients on daratumumab/pomalidomide/dexamethasone regimen (Nooka et al. 2016), and 21% in R/R patients with 1-15 lines of previous therapy on venetoclax monotherapy (n=66) (Kumar et al. 2016).

### Example 2. Lower Osteoclast Numbers in a Tumor Region is Associated with Clinical Efficacy of Anti-PD-L1 and Anti-CD38 Combination Treatment in Relapsed or Refractory Multiple Myeloma

Immune checkpoint inhibition targeting the PD-1/PD-L1 pathway is insufficient to induce clinical response in relapsed or refractory (R/R) multiple myeloma (MM). We postulated that combining atezolizumab (A; anti-PD-L1) with daratumumab (D; anti-CD38), which targets myeloma cells and has immunomodulatory activity, may alter the tumor microenvironment (TME) to favor cytotoxic T-cell activation and clinical activity. To assess the efficacy of this combination, we studied osteoclasts in daratumumab-naïve and daratumumab-refractory patients from a Phase Ib study (GO29695; NCT02431208)

To understand the mechanisms regulating sensitivity to treatment, we studied the spatial localization of osteoclasts with respect to CD138⁺ tumor cells by dual-plex immunohistochemistry (IHC) (CD138/osteoclast) using bone biopsies. Osteoclasts were enumerated based on TRAP positivity and morphology. The number of osteoclasts in the tumor region was higher in resistant patients, suggesting that these cells may contribute to the inhibition of T-cell function as reported (An et al 2016; 128; 1590-1603). This hypothesis was further supported by higher osteoclast numbers in daratumumab-refractory patients at baseline (Tables 4 and 5).

TABLE 4
Differences between responder and non-responder in patients treated with
atezolizumab (A) and daratumumab (D)

| | | | Periphery: | | BMA: | | | Baseline |
|---|---|---|---|---|---|---|---|---|
| | | | % CD8⁺HLA- | | % CD8⁺HLA- | | | IHC |
| | | | DR⁺Ki-67⁺ on | | DR⁺Ki-67⁺ on | | | osteoclast |
| | | D status | treatment vs | | treatment vs | | | number |
| | | (from | baseline | Response | baseline | | | (tumor |
| | | prior | Median | (to | Median | Baseline IHC CD8⁺ | | region) |
| | | line[s] of | change | A-D | change | T-cell density | | Median |
| Cohort | Treatment | treatment) | [95% CI] | treatment) | [95% CI] | (objects/mm² area) | | [95% CI] |
| D1, D2 | A-D | D-naïve | 11.9 [1.4, 15.7] | Yes | 4.6 [2.9, 4.6] | 510 [137, | 359 [241, | 3 [0, 17] |
| | | (n = 21) | (n = 7) | (n = 9) | (n = 3) | 639] | 605] | (n = 4) |
| D3 | A-D | D- | −0.3 [−1.2, 0.1] | No | 0.8 [−0.67, 0.9] | 190 [110, | 426 [199, | 30 [8, 40] |
| | | refractory | (n = 8) | (n = 12) | (n = 6) | 258] | 482] | (n = 13) |
| | | (n = 15) | | No | | (n = 11) | (n = 12) | |
| | | A-D | | (n = 15) | | | | |
| | | D3 | | | | | | |

TABLE 5
Differences between daratumumab (D)-naïve and D-refractory patients at baseline

| | | | | | | IHC osteoclast number |
|---|---|---|---|---|---|---|
| | | | | BMA MFI PD | BMA MFI PD-1 | (tumor region) |
| | | | D status (from | (CD8⁺Temra) | (CD8⁺Tem) | Median [95% CI] |
| | | Patients | prior line[s] of | Median [95% | Median [95% | 359 [241,605] |
| Cohort | Treatment | (n) | treatment) | CI] | CI] | (n = 4) |
| A | A | 6 | D-naïve | 1600 [1300, | 2710 [2120, 3190] | 10.5 [4, 18] |
| B | A-Len | 9 | (n = 43) | 1990] | (n = 26) | (n = 22) |
| E | A-D-Len | 7 | | (n = 26) | | |
| D1, D2 | A-D | 21 | | | | |
| D3 | A-D | 15 | D-refractory | 1250 [1020, | 1660 [1490, 1900] | 40 [8, 168] |
| | | | (n = 15) | 1390] | (n = 6) | (n = 7) |

A, atezolizumab;
D, daratumumab;
Len, lenalidomide;
Cohort A, D-naïve treated with A monotherapy;
Cohort B, D-naïve treated with A-Len;
Cohorts D1 and D2, D-naïve treated with A-D;
D3, D-refractory treated with A-D;
Cohort E, D-naïve treated with A-D-Len;
BMA, bone marrow aspirates;
MFI, median fluorescence intensity;
CD8⁺ T-effector cells (Temra, CD3⁺CD8⁺CD45RO−CCR7−);
CD8⁺ T-effector memory (Tem, CD3⁺CD8⁺CD45RO⁺CCR7⁻);
IHC, immunohistochemistry

### Example 3. Higher CD8⁺ Cell Density in Tumor Clusters is Associated with Clinical Efficacy of Anti-PD-L1 and Anti-CD38 Combination Treatment in Relapsed or Refractory Multiple Myeloma

To assess the efficacy of anti-PD-L1 and anti-CD38 combination treatment in relapsed or refractory multiple myeloma, we studied changes in CD8⁺ T cells in daratumumab-naïve and daratumumab-refractory patients.

Dual-plex immunohistochemistry (CD138/CD8, CD8/Ki-67) was performed using bone biopsies to study the spatial localization of CD8⁺ T cells with respect to CD138⁺ tumor cells. A higher density of CD8⁺ T cells within tumor clusters (CD138⁺ cell masses of >2000 μm²) was seen at baseline in sensitive versus resistant patients, but this was not observed outside of tumor clusters (Table 4).

### Example 4. An On-Treatment Increase in Activated CD8⁺ T-Cell Populations in the Bone Marrow is Associated with Treatment Responsiveness to Anti-PD-L1 and Anti-CD38 Combination Treatment in Relapsed or Refractory Multiple Myeloma

We studied CD8⁺ T-cell activation and proliferation (% CD8⁺HLA-DR⁺Ki-67⁺), the pharmacodynamic marker for atezolizumab (Herbst et al 2014; 515:563-567), using flow cytometry using longitudinal peripheral blood (PB) samples and using IHC (CD8/Ki-67) using longitudinal bone marrow biopsies. All daratumumab-naïve patients showed on treatment increase in % CD8⁺HLA-DR⁺Ki-67⁺ cells in the periphery (C1D15-C2D1) compared to baseline, which was not observed in daratumumab-refractory patients (Table 4). In BMA, the increase in % CD8⁺HLA-DR⁺Ki-67⁺ (C2D15-C4D1) was observed in daratumumab-naïve patients with clinical response to atezolizumab-daratumumab (sensitive), but not in non-responders (resistant) or daratumumab-refractory patients (all resistant), suggesting that sensitive patients have an immune-supportive TME. Preliminary IHC staining also showed an increase in CD8⁺Ki-67⁺ T cells in two responders after treatment.

Interestingly, higher median fluorescence intensity of PD-1 on CD8⁺ T-effector cells and on CD8+T-effector memory cells was observed at baseline in daratumumab-naïve relative to daratumumab-refractory patients, while the level of PD-L1 expression on tumor cells was similar. An increase in activated proliferating T cells (% CD8⁺HLA-DR⁺Ki-67+) observed after treatment in responders in daratumumab-naïve patients suggests that high PD-1 expression in this subset is not a marker of CD8⁺ T-cell exhaustion, but of functional capability (Table 5).

### Other Aspects

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method of identifying an individual having a hematologic cancer who may benefit from a treatment comprising a PD-L1 axis binding antagonist and an anti-CD38 antibody, the method comprising determining an osteoclast number in a tumor sample obtained from the individual, wherein an osteoclast number that is lower than a reference osteoclast number identifies the individual as one who may benefit from the treatment.

2. The method of claim 1, wherein the osteoclast number in the tumor sample is the number of osteoclasts within a tumor region.

3. The method of claim 2, wherein the tumor region comprises an area comprising tumor cells and adjacent myeloid cells.

4. The method of claim 2 or 3, wherein the tumor region does not comprise fat bodies and bone trabeculae.

5. The method of claim 3 or 4, wherein the tumor region comprises an area within about 40 μm to about 1 mm of a tumor cell or a myeloid cell adjacent to a tumor cell.

6. The method of any one of claims 1-5, wherein the osteoclast number in the tumor sample is lower than the reference osteoclast number and the method further comprises administering to the individual a treatment comprising a PD-L1 axis binding antagonist and an anti-CD38 antibody.

7. A method of treating an individual having a hematologic cancer, the method comprising:
(a) determining an osteoclast number in a tumor sample obtained from the individual, wherein the osteoclast number in the tumor sample has been determined to be lower than a reference osteoclast number; and
(b) administering an effective amount of a PD-L1 axis binding antagonist and an anti-CD38 antibody to the individual based on the osteoclast number in the tumor sample determined in step (a).

8. A method of treating an individual having a hematologic cancer, the method comprising administering to the individual an effective amount of a PD-L1 axis binding antagonist and an anti-CD38 antibody, wherein prior to treatment an osteoclast number in a tumor sample obtained from the individual has been determined to be lower than a reference osteoclast number.

9. The method of any one of claims 1-8, wherein the reference osteoclast number is a baseline osteoclast number in a reference population of individuals having the hematologic cancer, the reference population consisting of individuals who have been treated with a PD-L1 axis binding antagonist and an anti-CD38 antibody.

10. The method of claim 9, wherein the reference osteoclast number significantly separates a first subset of individuals from a second subset of individuals in the reference population based on a significant difference in responsiveness to treatment with the PD-L1 axis binding antagonist and the anti-CD38 antibody.

11. The method of any one of claims 1-10, wherein the reference osteoclast number is a pre-assigned osteoclast number.

12. A method of identifying an individual having a hematologic cancer who may benefit from a treatment comprising a PD-L1 axis binding antagonist and an anti-CD38 antibody, the method comprising determining a CD8⁺ T cell density in a tumor sample obtained from the individual, wherein a CD8⁺ T cell density that is higher than a reference CD8⁺ T cell density identifies the individual as one who is more likely to benefit from the treatment.

13. The method of claim 12, wherein the CD8⁺ T cell density in the tumor sample is the density of CD8⁺ T cells within a tumor cluster.

14. The method of claim 13, wherein the tumor cluster is an area comprising adjacent tumor cells.

15. The method of claim 13 or 14, wherein the tumor cluster is at least about 25 μm to about 400 μm in length along its longest axis.

16. The method of any one of claims 12-15, wherein the CD8⁺ T cell density in the tumor sample is higher than the reference CD8⁺ T cell density and the method further comprises administering to the individual a treatment comprising a PD-L1 axis binding antagonist and an anti-CD38 antibody.

17. A method of treating an individual having a hematologic cancer, the method comprising:
(a) determining a CD8⁺ T cell density in a tumor sample obtained from the individual, wherein the CD8⁺ T cell density in the tumor sample has been determined to be higher than a reference CD8⁺ T cell density; and
(b) administering an effective amount of a PD-L1 axis binding antagonist and an anti-CD38 antibody to the individual based on the CD8⁺ T cell density in the tumor sample determined in step (a).

18. A method of treating an individual having a hematologic cancer, the method comprising administering to the individual an effective amount of a PD-L1 axis binding antagonist and an anti-CD38 antibody, wherein prior to treatment a CD8⁺ T cell density in a tumor sample obtained from the individual has been determined to be higher than a reference CD8⁺ T cell density.

19. The method of any one of claims 12-18, wherein the reference CD8⁺ T cell density is a baseline density of CD8⁺ T cells within tumor clusters in a reference population of individuals having the hematologic cancer, the reference population consisting of individuals who have been treated with a PD-L1 axis binding antagonist and an anti-CD38 antibody.

20. The method of claim 19, wherein the reference CD8⁺ T cell density significantly separates a first subset of individuals from a second subset of individuals in the reference population based on a significant difference in responsiveness to treatment with the PD-L1 axis binding antagonist and the anti-CD38 antibody.

21. The method of any one of claims 12-20, wherein the reference CD8⁺ T cell density is a pre-assigned CD8⁺ T cell density.

22. The method of any one of claims 1-21, wherein the individual has not been previously administered a treatment comprising a PD-L1 axis binding antagonist.

23. The method of claim 22, wherein the individual has not been previously administered a treatment comprising a PD-L1 axis binding antagonist and an anti-CD38 antibody.

24. A method of monitoring responsiveness of an individual having a hematologic cancer to a treatment comprising a PD-L1 axis binding antagonist and an anti-CD38 antibody, the method comprising:
(a) determining, in a biological sample obtained from the individual at a time point following administration of the PD-L1 axis binding antagonist and the anti-CD38 antibody, the number of activated CD8⁺ T cells in the bone marrow; and
(b) comparing the number of activated CD8⁺ T cells in the biological sample to a reference number of activated CD8⁺ T cells, wherein an increase in the number of activated CD8⁺ T cells in the biological sample relative to the reference number of activated CD8⁺ T cells indicates that the individual is responding to the treatment.

25. The method of claim 24, wherein the number of activated CD8⁺ T cells in the biological sample is increased relative to the reference number of activated CD8⁺ T cells.

26. The method of claim 25, wherein the method comprises administering a further dose of the PD-L1 axis binding antagonist and the anti-CD38 antibody to the individual based on the increase in the number of activated CD8⁺ T cells in the biological sample determined in step (b).

27. The method of any one of claims 24-26, wherein the reference number of activated CD8⁺ T cells is (i) the number of activated CD8⁺ T cells in a biological sample from the individual obtained prior to administration of the PD-L1 axis binding antagonist and the anti-CD38 antibody, (ii) the number of activated CD8⁺ T cells in a biological sample obtained from the individual at a previous time point, wherein the previous time point is following administration of the PD-L1 axis binding antagonist and the anti-CD38 antibody; or (iii) a pre-assigned number of activated CD8⁺ T cells.

28. The method of any one of claims 24-27, wherein the biological sample is a bone marrow aspirate.

29. The method of any one of claims 10, 20, and 24-28, wherein responsiveness to treatment is in terms of an objective response.

30. The method of claim 29, wherein the objective response is a stringent complete response (sCR), a complete response (CR), a very good partial response (VGPR), a partial response (PR), or a minimal response (MR).

31. The method of any one of claims 1-30, wherein the hematologic cancer is a myeloma.

32. The method of claim 31, wherein the myeloma is a multiple myeloma (MM).

33. The method of claim 32, wherein the MM is a relapsed or refractory MM.

34. The method of any one of claims 1-33, wherein the anti-CD38 antibody is an anti-CD38 antagonist antibody.

35. The method of any one of claims 1-34, wherein the anti-CD38 antibody comprises the following complementarity determining regions (CDRs):
(a) a CDR-H1 comprising the amino acid sequence of SFAMS (SEQ ID NO: 1);
(b) a CDR-H2 comprising the amino acid sequence of AISGSGGGTYYADSVKG (SEQ ID NO: 2);
(c) a CDR-H3 comprising the amino acid sequence of DKILWFGEPVFDY (SEQ ID NO: 3);
(d) a CDR-L1 comprising the amino acid sequence of RASQSVSSYLA (SEQ ID NO: 4);
(e) a CDR-L2 comprising the amino acid sequence of DASNRAT (SEQ ID NO: 5); and
(f) a CDR-L3 comprising the amino acid sequence of QQRSNWPPTF (SEQ ID NO: 6).

36. The method of claim 35, wherein the anti-CD38 antibody comprises the following light chain variable region framework regions (FRs):
(a) an FR-L1 comprising the amino acid sequence of EIVLTQSPATLSLSPGERATLSC (SEQ ID NO: 7);
(b) an FR-L2 comprising the amino acid sequence of WYQQKPGQAPRLLIY (SEQ ID NO: 8);
(c) an FR-L3 comprising the amino acid sequence of GIPARFSGSGSGTDFTLTISSLEPEDFAVYYC (SEQ ID NO: 9); and
(d) an FR-L4 comprising the amino acid sequence of GQGTKVEIK (SEQ ID NO: 10).

37. The method of claim 36, wherein the anti-CD38 antibody comprises the following heavy chain variable region FRs:
(a) an FR-H1 comprising the amino acid sequence of EVQLLESGGGLVQPGGSLRLSCAVSGFTFN (SEQ ID NO: 11);
(b) an FR-H2 comprising the amino acid sequence of WVRQAPGKGLEWVS (SEQ ID NO: 12);
(c) an FR-H3 comprising the amino acid sequence of RFTISRDNSKNTLYLQMNSLRAEDTAVYFCAK (SEQ ID NO: 13); and
(d) an FR-H4 comprising the amino acid sequence of WGQGTLVTVSS (SEQ ID NO: 14).

38. The method of any one of claims 35-37, wherein the anti-CD38 antibody comprises:
(a) a heavy chain variable (V_{H}) domain comprising an amino acid sequence having at least 95% sequence identity to the amino acid sequence of
(SEQ ID NO: 15)
EVQLLESGGGLVQPGGSLRLSCAVSGFTFNSFAMSWVRQAPGKGLEWVS
AISGSGGGTYYADSVKGRFTISRDNSKNTLYLQMNSLRAEDTAVYFCAK
DKILWFGEPVFDYWGQGTLVTVSS;
(b) a light chain variable (V_{L}) domain comprising an amino acid sequence having at least 95% sequence identity to the amino acid sequence of
(SEQ ID NO: 16)
EIVLTQSPATLSLSPGERATLSCRASQSVSSYLAWYQQKPGQAPRLLIY
DASNRATGIPARFSGSGSGTDFTLTISSLEPEDFAVYYCQQRSNWPPTF
GQGTKVEIK;
or
(c) a V_{H} domain as in (a) and a V_{L} domain as in (b).

39. The method of claim 38, wherein the anti-CD38 antibody comprises:
(a) a V_{H} domain comprising the amino acid sequence of SEQ ID NO: 15; and
(b) a V_{L} domain comprising the amino acid sequence of SEQ ID NO: 16.

40. The method of any one of claims 1-39, wherein the anti-CD38 antibody is a monoclonal antibody.

41. The method of any one of claims 1-40, wherein the anti-CD38 antibody is a human antibody.

42. The method of any one of claims 1-41, wherein the anti-CD38 antibody is a full-length antibody.

43. The method of any one of claims 1-42, wherein the anti-CD38 antibody is daratumumab.

44. The method of any one of claims 1-41, wherein the anti-CD38 antibody is an antibody fragment that binds CD38 selected from the group consisting of Fab, Fab′, Fab′-SH, Fv, single chain variable fragment (scFv), and (Fab′)₂ fragments.

45. The method of any one of claims 1-44, wherein the anti-CD38 antibody is an IgG class antibody.

46. The method of claim 45, wherein the IgG class antibody is an IgG1 subclass antibody.

47. The method of any one of claims 6-11 and 16-46, wherein the method comprises administering to the individual the anti-CD38 antibody intravenously.

48. The method of any one of claims 6-11 and 16-47, wherein the method comprises administering to the individual the anti-CD38 antibody at a dose of about 16 mg/kg.

49. The method of any one of claims 1-48, wherein the PD-L1 axis binding antagonist is selected from the group consisting of a PD-L1 binding antagonist, a PD-1 binding antagonist, and a PD-L2 binding antagonist.

50. The method of claim 49, wherein the PD-L1 axis binding antagonist is a PD-L1 binding antagonist.

51. The method of claim 50, wherein the PD-L1 binding antagonist inhibits the binding of PD-L1 to one or more of its ligand binding partners.

52. The method of claim 51, wherein the PD-L1 binding antagonist inhibits the binding of PD-L1 to PD-1, B7-1, or both PD-1 and B7-1.

53. The method of any one of claims 49-52, wherein the PD-L1 binding antagonist is an anti-PD-L1 antibody.

54. The method of claim 53, wherein the anti-PD-L1 antibody is atezolizumab (TECENTRIQ®), MDX-1105, MEDI4736 (durvalumab), or MSB0010718C (avelumab).

55. The method of claim 54, wherein the anti-PD-L1 antibody is atezolizumab.

56. The method of any one of claims 53-55, wherein the anti-PD-L1 antibody comprises the following hypervariable regions (HVRs):
(a) an HVR-H1 sequence of
(SEQ ID NO: 17)
GFTFSDSWIH;
(b) an HVR-H2 sequence of
(SEQ ID NO: 18)
AWISPYGGSTYYADSVKG;
(c) an HVR-H3 sequence of
(SEQ ID NO: 19)
RHWPGGFDY;
(d) an HVR-L1 sequence of
(SEQ ID NO: 20)
RASQDVSTAVA;
(e) an HVR-L2 sequence of
(SEQ ID NO: 21)
SASFLYS;
and
(f) an HVR-L3 sequence of
(SEQ ID NO: 22)
QQYLYHPAT.

57. The method of any one of claims 53-56, wherein the anti-PD-L1 antibody comprises:
(a) a heavy chain variable (V_{H}) domain comprising an amino acid sequence having at least 90% sequence identity to the amino acid sequence of SEQ ID NO: 23;
(b) a light chain variable (V_{L}) domain comprising an amino acid sequence having at least 90% sequence identity to the amino acid sequence of SEQ ID NO: 24; or
(c) a V_{H} domain as in (a) and a V_{L} domain as in (b).

58. The method of claim 57, wherein the anti-PD-L1 antibody comprises:
(a) a V_{H} domain comprising an amino acid sequence having at least 95% sequence identity to the amino acid sequence of SEQ ID NO: 23;
(b) a V_{L} domain comprising an amino acid sequence having at least 95% sequence identity to the amino acid sequence of SEQ ID NO: 24; or
(c) a V_{H} domain as in (a) and a V_{L} domain as in (b).

59. The method of claim 58, wherein the anti-PD-L1 antibody comprises:
(a) a V_{H} domain comprising an amino acid sequence having at least 95% sequence identity to the amino acid sequence of SEQ ID NO: 23;
(b) a V_{L} domain comprising an amino acid sequence having at least 95% sequence identity to the amino acid sequence of SEQ ID NO: 24; or
(c) a V_{H} domain as in (a) and a V_{L} domain as in (b).

60. The method of claim 59, wherein the anti-PD-L1 antibody comprises:
(a) a V_{H} domain comprising an amino acid sequence having at least 96% sequence identity to the amino acid sequence of SEQ ID NO: 23;
(b) a V_{L} domain comprising an amino acid sequence having at least 96% sequence identity to the amino acid sequence of SEQ ID NO: 24; or
(c) a V_{H} domain as in (a) and a V_{L} domain as in (b).

61. The method of claim 60, wherein the anti-PD-L1 antibody comprises:
(a) a V_{H} domain comprising an amino acid sequence having at least 97% sequence identity to the amino acid sequence of SEQ ID NO: 23;
(b) a V_{L} domain comprising an amino acid sequence having at least 97% sequence identity to the amino acid sequence of SEQ ID NO: 24; or
(c) a V_{H} domain as in (a) and a V_{L} domain as in (b).

62. The method of claim 61, wherein the anti-PD-L1 antibody comprises:
(a) a V_{H} domain comprising an amino acid sequence having at least 98% sequence identity to the amino acid sequence of SEQ ID NO: 23;
(b) a V_{L} domain comprising an amino acid sequence having at least 98% sequence identity to the amino acid sequence of SEQ ID NO: 24; or
(c) a V_{H} domain as in (a) and a V_{L} domain as in (b).

63. The method of claim 62, wherein the anti-PD-L1 antibody comprises:
(a) a V_{H} domain comprising an amino acid sequence having at least 99% sequence identity to the amino acid sequence of SEQ ID NO: 23;
(b) a V_{L} domain comprising an amino acid sequence having at least 99% sequence identity to the amino acid sequence of SEQ ID NO: 24; or
(c) a V_{H} domain as in (a) and a V_{L} domain as in (b).

64. The method of claim 63, wherein the anti-PD-L1 antibody comprises:
(a) a V_{H} domain comprising the amino acid sequence of SEQ ID NO: 23;
(b) a V_{L} domain comprising the amino acid sequence of SEQ ID NO: 24; or
(c) a V_{H} domain as in (a) and a V_{L} domain as in (b).

65. The method of claim 64, wherein the anti-PD-L1 antibody comprises:
(a) a V_{H} domain comprising the amino acid sequence of SEQ ID NO: 23; and
(b) a V_{L} domain comprising the amino acid sequence of SEQ ID NO: 24.

66. The method of any one of claims 6-11 and 16-65, wherein the method comprises administering to the individual the PD-L1 axis binding antagonist intravenously.

67. The method of claim 66, wherein the PD-L1 axis binding antagonist is atezolizumab.

68. The method of claim 67, wherein atezolizumab is administered to the individual intravenously at a dose of about 840 mg every 2 weeks, about 1200 mg every 3 weeks, or about 1680 mg of every 4 weeks.

69. The method of claim 68, wherein atezolizumab is administered to the individual intravenously at a dose of about 1200 mg every 3 weeks.

70. The method of claim 69, wherein atezolizumab is administered to the individual intravenously at a dose of about 1200 mg on Day −2 to Day 4 of one or more 21-day dosing cycles.

71. The method of claim 70, wherein atezolizumab is administered to the individual intravenously at a dose of about 1200 mg on Day 1 of each 21-day dosing cycle.

72. The method of claim 49, wherein the PD-L1 axis binding antagonist is a PD-1 binding antagonist.

73. The method of claim 72, wherein the PD-1 binding antagonist inhibits the binding of PD-1 to one or more of its ligand binding partners.

74. The method of claim 73, wherein the PD-1 binding antagonist inhibits the binding of PD-1 to PD-L1, PD-L2, or both PD-L1 and PD-L2.

75. The method of any one of claims 49 and 72-74, wherein the PD-1 binding antagonist is an anti-PD-1 antibody.

76. The method of claim 75, wherein the anti-PD-1 antibody is MDX-1106 (nivolumab), MK-3475 (pembrolizumab), MEDI-0680 (AMP-514), PDR001, REGN2810, or BGB-108.

77. The method of any one of claims 49 and 72-74, wherein the PD-1 binding antagonist is an Fc fusion protein.

78. The method of claim 77, wherein the Fc fusion protein is AMP-224.

79. The method of any one of claims 1-78, wherein the individual is a human.
